SEQUENCE LISTING (6 sequences)

SEQ ID NO: 1 - DNA, 25 bp, Artificial Sequence
  Synthetic
ggucauccaugacaacuuugguatc

SEQ ID NO: 2 - RNA, 27 nt, Artificial Sequence
  Synthetic
gauaccaaaguugucauggaugaccuu

SEQ ID NO: 3 - RNA, 21 nt, Artificial Sequence
  Synthetic
gaauguggguggcaacuuuag

SEQ ID NO: 4 - RNA, 21 nt, Artificial Sequence
  Synthetic
aaaguugccacccacauucag

SEQ ID NO: 5 - DNA, 25 bp, Artificial Sequence
  Synthetic
gaauguggguggcaacuuuaaagga

SEQ ID NO: 6 - RNA, 27 nt, Artificial Sequence
  Synthetic
uccuuuaaaguugccacccacauucag

(19) United States
(12) Patent Application Publication
(10) Pub. No.: US 2011/0142951 A1
(43) Pub. Date: Jun. 16, 2011

(54) MICELLES FOR INTRACELLULAR DELIVERY OF THERAPEUTIC AGENTS

(75) Inventors: Paul H. Johnson, Snohomish, WA (US); Patrick S. Stayton, Seattle, WA (US); Allan S. Hoffman, Seattle, WA (US); Anthony J. Convertine, Seattle, WA (US); Robert W. Overell, Shoreline, WA (US); Anna S. Gall, Woodinville, WA (US); Mary G. Prieve, Lake Forest Park, WA (US); Amber E.E. Paschal, Redmond, WA (US); Charbel Diab, Seattle, WA (US); Priyadarsi De, Mohanpur (IN)

(73) Assignees: UNIVERSITY OF WASHINGTON, Seattle, WA (US); PHASERX, INC., Seattle, WA (US)

(21) Appl. No.: 12/992,541

(22) PCT Filed: May 13, 2009
(86) PCT No.: PCT/US09/43853

§ 371 Date: Feb. 8, 2011

Related U.S. Application Data
(60) Provisional application No. 61/140,779, filed on Dec. 24, 2008.
(60) Provisional application No. 61/112,048, filed on Nov. 6, 2008.
(60) Provisional application No. 61/140,774, filed on Dec. 24, 2008.
(60) Provisional application No. 61/091,294, filed on Aug. 22, 2008.
(60) Provisional application No. 61/052,908, filed on May 13, 2008.
(60) Provisional application No. 61/112,054, filed on Nov. 6, 2008.
(60) Provisional application No. 61/171,369, filed on Apr. 21, 2009.
(60) Provisional application No. 61/052,914, filed on May 13, 2008.
(60) Provisional application No. 61/171,358, filed on Apr. 21, 2009.

(51) Int. Cl. A61K 9/14; A61K 31/7088; C12N 5/02

(52) U.S. Cl. 424/501; 514/44 R; 435/375

(57) ABSTRACT

Composition comprising a polymeric micelle and an associated polynucleotide.

This application claims the benefit of U.S. Provisional Application No. 61/052,908, filed May 13, 2008, U.S. Provisional Application No. 61/052,914, filed May 13, 2008, U.S. Provisional Application No. 61/091,294, filed Aug. 22, 2008, U.S. Provisional Application No. 61/112,048, filed Nov. 6, 2008, U.S. Provisional Application No. 61/140,774, filed Dec. 24, 2008, and U.S. Provisional Application No. 61/171,369, filed Apr. 21, 2009, U.S. Provisional Application No. 61/140,779 filed Dec. 24, 2008, U.S. Provisional Application No. 61/112,054 filed Nov. 6, 2008, U.S. Provisional Application No. 61/171,358 filed Apr. 21, 2009, each of which is hereby incorporated by reference in its entirety.

### FIELD OF THE INVENTION

Described herein are micelles formed from polymers and the use of such micelles.

### BACKGROUND OF THE INVENTION

In certain instances, it is beneficial to provide therapeutic agents, such as polynucleotides (e.g., oligonucleotides) to living cells. In some instances, delivery of such polynucleotides to a living cell provides a therapeutic benefit.

### SUMMARY OF THE INVENTION

Provided herein are micelles for intracellular delivery of therapeutic agents (e.g., oligonucleotides, peptides or the like). In some embodiments, such intracellular delivery is in vitro; in other embodiments, such intracellular delivery is in vivo.

In some embodiments micelles provided herein are specifically designed for targeted delivery of a micellar payload at a desired site of therapeutic intervention in a subject. Accordingly, the micelle is preferably stable to dilution at physiologic pH. In some embodiments, the micelles provided herein are stable under physiological conditions and have critical micellar concentrations that prevent undesired dissociation of the micelle. In further or alternative embodiments, the block copolymers comprising the micelles described herein have block ratios, block sizes and/or core properties and/or shell properties that are designed for enhanced micellar integrity under physiological conditions. In further or alternative embodiments, the integrity of a micelle in the physiological milieu is also dependent on the composition of the block copolymers that comprise a micelle. Accordingly, provided herein are certain parameters (e.g., the number average molecular weight ratios for block copolymers in the shell block and the core block of micelles, number of charged moieties in the block copolymers, and the like) that are engineered to provide micelles suitable for efficient intracellular delivery of therapeutic agents with minimal toxicity and/or loss of micellar payload.

Provided in some embodiments, is composition comprising a polymeric micelle and a polynucleotide associated with the micelle, the micelle comprising a plurality of block copolymers, each block copolymer comprising a hydrophilic block and a hydrophobic block, the plurality of block copolymers associating such that the micelle is stable in an aqueous medium at about neutral pH,
(a) the micelle further having two or more characteristics selected from:
   (i) the micelle comprising from about 10 to about 100 of the block copolymers per micelle,
   (ii) a critical micelle concentration, CMC, ranging from about 0.2 μg/mL to about 20 μg/mL,
   (iii) spontaneous micelle assembly in the absence of nucleic acid;
   (iv) a weight average molecular weight of about 0.5×10⁶ to about 3.6×10⁶ dalton;
   (v) a particle size of about 5 nm to about 500 nm; and
(b) the block copolymers having one or more characteristic selected from:
   (i) a ratio of a number-average molecular weight, Mₙ, of the hydrophilic block to the hydrophobic block, ranging from about 1:1 to about 1:10, and
   (ii) a polydispersity index of about 1.0 to about 2.0.

Provided, in some embodiments, is a composition comprising a polymeric micelle and a polynucleotide associated with the micelle, the micelle comprising a plurality of block copolymers, each block copolymer comprising a hydrophilic block and a hydrophobic block, the plurality of block copolymers associating such that the micelle is stable in an aqueous medium at about neutral pH,
(a) the micelle further having two or more characteristics selected from:
   (i) the micelle comprising from about 10 to about 100 of the block copolymers per micelle,
   (ii) a critical micelle concentration, CMC, ranging from about 0.2 μg/mL to about 20 μg/mL in 0.5 M NaCl;
   iii) spontaneous micelle assembly in the absence of nucleic acid;
   (iv) a weight average molecular weight of about 0.5×10⁶ to about 3.6×10⁶ dalton; and
(b) the block copolymers having one or more characteristic selected from:
   (i) a ratio of a number-average molecular weight, Mₙ, of the hydrophilic block to the hydrophobic block, ranging from about 1:1 to about 1:10, and
   (ii) a polydispersity index of about 1.0 to about 2.0.

Provided, in some embodiments, is a composition comprising a polymeric micelle and a polynucleotide associated with the micelle, the micelle comprising a plurality of block copolymers, each block copolymer comprising a hydrophilic block and a hydrophobic block, the plurality of block copolymers associating such that the micelle is stable in an aqueous medium at about neutral pH, the micelle further having two or more characteristics selected from:
(i) an association number ranging from about 10 to about 100 chains per micelle,
(ii) a critical micelle concentration, CMC, ranging from about 0.2 μg/mL to about 20 μg/mL,
(iii) a particle size of about 5 nm to about 500 nm.

Provided, in some embodiments, is a composition comprising a polymeric micelle and a polynucleotide associated with the micelle, the micelle comprising a plurality of block copolymers, each block copolymer comprising a hydrophilic block and a hydrophobic block, the plurality of block copolymers associating such that the micelle is stable in an aqueous medium at about neutral pH, the block copolymers having two or more characteristics selected from:
(i) a ratio of a number-average molecular weight, Mₙ, of the hydrophilic block to the hydrophobic block, ranging from about 1:1 to about 1:10,
(ii) a polydispersity index of about 1.0 to about 2.0, and
(iii) a weight average molecular weight of about 0.5×10⁶ to about 3.6×10⁶ g/mol.

In certain embodiments, the composition comprises a micelle that has three or more of the characteristics of subparagraphs (i), (ii), (iii), (iv) and (v) thereof. In certain embodiments, the micelle is has all of the characteristics of subparagraphs (i), (ii), (iii) (iv) and (v) thereof.

In certain embodiments, the composition comprises a block copolymer that has all of the characteristics of subparagraphs (i), (ii), and (iii) thereof. In some embodiments, the block copolymer has a ratio of a number-average molecular weight, Mn, of the hydrophilic block to the hydrophobic block, ranging from about 1:1 to about 1:10. In some embodiments, the block copolymer has a ratio of a number-average molecular weight, Mn, of the hydrophilic block to the hydrophobic block, ranging from about 1:1.5 to about 1:6. In certain embodiments, the block copolymer has a ratio of a number-average molecular weight, Mn, of the hydrophilic block to the hydrophobic block, ranging from about 1:2 to about 1:4.

In some embodiments, the composition comprises a micelle that comprises about 10 to about 100 of the block copolymers per micelle. In some embodiments, the micelle comprises about 20 to about 60 of the block copolymers per micelle. In some embodiments, the micelle is comprises about 30 to about 50 of the block copolymers per micelle.

In some embodiments, the composition comprises a micelle that has a critical micelle concentration, CMC, of about 0.2 μg/mL to about 20 μg/mL. In some embodiments, the micelle has a critical micelle concentration, CMC, of about 0.5 μg/mL to about 10 μg/mL. In some embodiments, the micelle has a critical micelle concentration, CMC, of about 1 μg/mL to about 5 μg/mL.

In some embodiments, the composition comprises a block copolymer having a ratio of a number-average molecular weight, Mn, of the hydrophilic block to the hydrophobic block, ranging from about 1:1.5 to about 1:6; and the micelle
(i) comprises about 20 to about 60 of the block copolymers per micelle, and
(ii) has a critical micelle concentration, CMC, of about 0.5 μg/mL to about 10 μg/mL.

In some embodiments, the block copolymer has a ratio of a number-average molecular weight, Mn, of the hydrophilic block to the hydrophobic block, ranging from about 1:2 to about 1:4; and the micelle:
(i) comprises about 30 to about 50 of the block copolymers per micelle, and
(ii) has a critical micelle concentration, CMC, ranging from about 1 ug/mL to about 5 ug/mL.

In some embodiments, the block copolymers described herein have a polydispersity index of about 1.0 to about 2.0. In some embodiments, the block copolymers have a polydispersity index of about 1.0 to about 1.7. In some embodiments, the block copolymers have a polydispersity index of about 1.0 to about 1.4.

In some embodiments, a composition provided herein comprises a micelle having an aggregate molecular weight, M_{w}, of about 0.5×10⁶ to about 3.6×10⁶. In some embodiments, the micelle has an aggregate molecular weight, M_{w}, of about 0.75×10⁶ to about 2.0×10⁶. In some embodiments, the micelle has an aggregate molecular weight, M_{w}, of about 1.0×10⁶ to about 1.5×10⁶.

In some embodiments, the micelle has a particle size of about 5 nm to about 500 nm. In some embodiments, the micelle has a particle size of about 10 nm to about 200 nm. In some embodiments, the micelle has a particle size of about 20 nm to about 100 nm.

In some embodiments of compositions provided herein, the number of polynucleotides associated with each micelle is about 1 to about 10,000. In some embodiments, the number of polynucleotides associated with each micelle is about 4 to about 5,000. In some embodiments, the number of polynucleotides associated with each micelle is about 15 to about 3,000. In some embodiments, the number of polynucleotides associated with each micelle is about 30 to about 2,500.

In some embodiments, a micelle described herein comprises a block copolymer comprising a plurality of cationic monomeric units, the cationic species in the hydrophilic block being in ionic association with the polynucleotide. In some embodiments, the cationic monomeric units are residues of cationic monomers, non-charged Brønsted base monomers, or a combination thereof.

In some embodiments of compositions provided herein, the polynucleotide is a RNAi agent or an siRNA In some embodiments, the polynucleotide is not in the core of the micelle

In some embodiments, a micelle described herein comprises a block copolymer comprising a plurality of anionic monomeric units in the hydrophilic block and/or the hydrophobic block.

In some embodiments, the micelle comprises a block copolymer comprising a plurality of uncharged monomeric units in the hydrophilic block and/or the hydrophobic block.

In some embodiments, the micelle comprises a block copolymer comprising a plurality of zwitterionic monomeric units in the hydrophilic block and/or the hydrophobic block.

In some embodiments, the micelle comprises a block copolymer comprising a plurality of chargeable residues in the hydrophobic block. In some embodiments, the micelle comprises a block copolymer comprising at least 20 chargeable residues in the hydrophobic block. In some embodiments, the micelle comprises a block copolymer comprising at least 15 chargeable residues in the hydrophobic block. In some embodiments, the micelle comprises a block copolymer comprising at least 10 chargeable residues in the hydrophobic block. In some embodiments, the micelle comprises a block copolymer comprising at least 5 chargeable residues in the hydrophobic block.

In some embodiments, a composition described herein comprises a polymer bioconjugate comprising one or more polynucleotides covalently coupled to one or more of the plurality of block copolymers. In some embodiments, the polynucleotide is an siRNA

In some embodiments, a micelle described herein comprises a block copolymer comprising a plurality of monomeric units having a protonatable anionic species and a plurality of hydrophobic species. In some embodiments, the anionic monomeric units are residues of anionic monomers, non charged Brønsted acid monomers, or a combination thereof.

In some embodiments, the micelle comprises a block copolymer comprising a plurality of monomeric units derived from a polymerizable monomer having a hydrophobic species.

In some embodiments, the block copolymer is a membrane destabilizing block copolymer.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: An illustrative example of the composition and properties of RAFT synthesized polymers

FIG. 2: An illustrative example of the synthesis of [PEGMA_{w}]-[B—P-D] polymers

FIG. 3: An illustrative example of the composition and properties of RAFT synthesized polymers

FIG. 4: An illustrative example of the composition and properties of PEGMA-DMAEMA copolymers

FIG. 5: An illustrative example of the synthesis of [PEGMA_{w}-MAA(NHS)]—[B—P-D] polymers

FIG. 6: An illustrative example of the composition and properties of RAFT synthesized polymers

FIG. 7: An illustrative example of the composition and properties of RAFT synthesized polymers

FIG. 8: Synthesis of PDSMA

FIG. 9: Synthesis of HPMA-PDSMA co-polymer for siRNA conjugation

FIG. 10: An illustrative example of the NMR spectroscopy of block copolymer PRx0729v6.

FIG. 11: An illustrative example of the polymer PRx0729v6 particle stability in organic solvents.

FIG. 12: An illustrative transmission electron microscopy (TEM) analysis of polymer PRx0729v6.

FIG. 13: An illustrative example of the effect of pH on polymer structure.

FIG. 14: An illustrative example of the critical stability concentration (CSC) of polymer PRx0729v6.

FIG. 15: An illustrative example of the dynamic light scattering (DLS) determination of particle size of polymer PRx0729v6 complexed to siRNA.

FIG. 16: An illustrative example of the gel shift analysis of polymer PRx0729v6/siRNA complexes at different charge ratios.

FIG. 17: An illustrative example of the knock-down activity of siRNA—micelle complexes in cultured mammalian cells.

FIG. 18: An illustrative example of the knock-down activity of siRNA—micelle complexes in cultured mammalian cells.

FIG. 19: An illustrative demonstration of membrane destabilizing activity of polymeric micelles and their siRNA complexes.

FIG. 20: An illustrative fluorescence microscopy of cell uptake and intracellular distribution of polymer-siRNA complexes.

FIG. 21: An illustrative example of the galactose end functionalized poly[DMAEMA]-macro CTA

FIG. 22: An illustrative example of the galactose functionalized DMAEMA-MAA(NHS) or PEGMA-MAA(NHS) di-block co-polymers

FIG. 23: An illustrative example of the structures of conjugatable siRNAs and pyridyl disulfide amine

### DETAILED DESCRIPTION OF THE INVENTION

Provided in certain embodiments herein are compositions comprising a polymeric micelle and a polynucleotide associated with the micelle, the micelle comprising a plurality of block copolymers. Generally, each block copolymer comprises a hydrophilic block and a hydrophobic block. In certain embodiments, the polymeric micelles described herein associate in such a manner so as to be stable in an aqueous medium, e.g., at about neutral pH.

In some embodiments, block copolymers comprising a micelle comprise a shell block and a core block. In some embodiments, the micelles described herein comprise a hydrophobic core and a hydrophilic shell. In some embodiments, the micelles described herein are self-assembled. In some embodiments, the micelles formation occurs in the absence of a polynucleotide. In some embodiments, micelle formation occurs in the presence of a polynucleotide. In specific embodiments, the micelles described herein are spontaneously self-assembled.

In certain embodiments, the core of the micelle comprises a plurality of hydrophobic groups. In some embodiments, the hydrophobic groups are hydrophobic at about a neutral pH. In more specific embodiments, the hydrophobic groups are more hydrophobic at a slightly acidic pH (e.g., at a pH of about 6 and/or a pH of about 5). In certain embodiments, two, four, ten, fifteen, twenty or more hydrophobic groups are present on a polymer block that together with other similar polymer blocks can form the core of the micelle. In some embodiments, a hydrophobic group has a π value of about one, or more. A compound's π value is a measure of its relative hydrophilic-lipophilic value (see, e.g., Cates, L. A., “Calculation of Drug Solubilities by Pharmacy Students” Am. J. Pharm. Educ. 45:11-13 (1981)).

In specific embodiments, the shell block is hydrophilic (e.g., at about a neutral pH). In some embodiments, the micelle is destabilized or disassociated at a pH within about 4.7 to about 6.8.

In some instances, provided herein are micellar compositions suitable for the delivery of therapeutic agents (including, e.g., oligonucleotides or peptides) to a living cell. In some embodiments, the micelles comprise a plurality of block copolymers and, optionally, at least one therapeutic agent. In certain embodiments, the micelles provided herein are biocompatible, stable (including chemically and/or physically stable), and/or reproducibly synthesized. Additionally, in some embodiments, the micelles assemblies provided herein are non-toxic (e.g., exhibit low toxicity), protect the therapeutic agent (e.g., oligonucleotide or peptide) payload from degradation, enter living cells via a naturally occurring process (e.g., by endocytosis), and/or deliver the therapeutic agent (e.g., oligonucleotide or peptide) payload into the cytoplasm of a living cell after being contacted with the cell. In certain instances, the polynucleotide (e.g., oligonucleotide) is an siRNA and/or another ‘nucleotide-based’ agent that alters the expression of at least one gene in the cell. Accordingly, in certain embodiments, the micelles provided herein are useful for delivering siRNA or peptide into a cell. In certain instances, the cell is in vitro, and in other instances, the cell is in vivo (e.g., a human subject). In some embodiments, a therapeutically effective quantity of the micelles comprising an siRNA or peptide is administered to an individual in need thereof (e.g., in need of having a gene knocked down, wherein the gene is capable of being knocked down by the siRNA administered). In specific instances, the micellar compositions described herein are useful for or are specifically designed for delivery of siRNA or peptide to specifically targeted cells of an individual.

### DEFINITIONS

It is understood that, with regard to this application, use of the singular includes the plural and vice versa unless expressly stated to be otherwise. That is, “a” and “the” refer to one or more of whatever the word modifies. For example, “the polymer” or “a nucleotide” may refer to one polymer or nucleotide or to a plurality of polymers or nucleotides. By the same token, “polymers” and “nucleotides” would refer to one polymer or one nucleotide as well as to a plurality of polymers or nucleotides unless, again, it is expressly stated or obvious from the context that such is not intended.

As used herein, two moieties or compounds are “attached” if they are held together by any interaction including, by way of non-limiting example, one or more covalent bonds, one or more non-covalent interactions (e.g., ionic bonds, static forces, van der Waals interactions, combinations thereof, or the like), or a combination thereof.

Aliphatic or aliphatic group: the term “aliphatic” or “aliphatic group”, as used herein, means a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-20 carbon atoms.

Anionic monomer: “Anionic monomer” or “anionic monomeric unit”, as used herein, is a monomer or monomeric unit bearing a group that is present in an anionic charged state or in a non-charged state, but in the non-charged state is capable of becoming anionic charged, e.g., upon removal of an electrophile (e.g., a proton (H+), for example in a pH dependent manner). In certain instances, the group is substantially negatively charged at an approximately physiological pH but undergoes protonation and becomes substantially neutral at a weakly acidic pH. The non-limiting examples of such groups include carboxyl groups, barbituric acid and derivatives thereof, xanthine and derivatives thereof, boronic acids, phosphinic acids, phosphonic acids, sulfinic acids, phosphates, and sulfonamides.

Anionic species: “Anionic species”, as used herein, is a group, residue or molecule that is present in an anionic charged or non-charged state, but in the non-charged state is capable of becoming anionic charged, e.g., upon removal of an electrophile (e.g., a proton (H+), for example in a pH dependent manner). In certain instances, the group, residue or molecule is substantially negatively charged at an approximately physiological pH but undergoes protonation and becomes substantially neutral at a weakly acidic pH.

Aryl or aryl group: as used herein, the term “aryl” or “aryl group” refers to monocyclic, bicyclic, and tricyclic ring systems having a total of five to fourteen ring members, wherein at least one ring in the system is aromatic and wherein each ring in the system contains three to seven ring members.

Heteroalkyl: the term “heteroalkyl” means an alkyl group wherein at least one of the backbone carbon atoms is replaced with a heteroatom.

Heteroaryl: the term “heteroaryl” means an aryl group wherein at least one of the ring members is a heteroatom.

Heteroatom: the term “heteroatom” means an atom other than hydrogen or carbon, such as oxygen, sulfur, nitrogen, phosphorus, boron, arsenic, selenium or silicon atom.

As used herein, a micelle is “disrupted” if it does not function in an identical, substantially similar or similar manner and/or possess identical, substantially similar or similar physical and/or chemical characteristics as would a stable micelle. In “disruption” of a micelle can be determined in any suitable manner. In one instance, a micelle is “disrupted” if it does not have a hydrodynamic particle size that is less than 5 times, 4 times, 3 times, 2 times, 1.8 times, 1.6 times, 1.5 times, 1.4 times, 1.3 times, 1.2 times, or 1.1 times the hydrodynamic particle size of a micelle comprising the same block copolymers and as formed in an aqueous solution at a pH of 7.4, or formed in human serum. In one instance, a micelle is “disrupted” if it does not have a concentration of assembly that is less than 5 times, 4 times, 3 times, 2 times, 1.8 times, 1.6 times, 1.5 times, 1.4 times, 1.3 times, 1.2 times, or 1.1 times the concentration of assembly of a micelle comprising the same block copolymers and as formed in an aqueous solution at a pH of 7.4, or formed in human serum.

As used herein, a “chargeable species”, “chargeable group”, or “chargeable monomeric unit” is a species, group or monomeric unit in either a charged or non-charged state. In certain instances, a “chargeable monomeric unit” is one that can be converted to a charged state (either an anionic or cationic charged state) by the addition or removal of an electrophile (e.g., a proton (H⁺), for example in a pH dependent manner). The use of any of the terms “chargeable species”, “chargeable group”, or “chargeable monomeric unit” includes the disclosure of any other of a “chargeable species”, “chargeable group”, or “chargeable monomeric unit” unless otherwise stated. A “chargeable species” that is “charged or chargeable to an anion” or “charged or chargeable to an anionic species” is a species or group that is either in an anionic charged state or non-charged state, but in the non-charged state is capable of being converted to an anionic charged state, e.g., by the removal of an electrophile, such as a proton (H+). In specific embodiments, a chargeable species is a species that is charged to an anion at about neutral pH. It should be emphasized that not every chargeable species on a polymer will be anionic at a pH near the pKₐ (acid dissociation constant) of the chargeable species, but rather an equilibrium of anionic and non-anionic species will co-exist. A “chargeable species” that is “charged or chargeable to a cation” or “charged or chargeable to a cationic species” is a species or group that is either in an cationic charged state or non-charged state, but in the non-charged state is capable of being converted to a cationic charged state, e.g., by the addition of an electrophile, such as a proton (H+). In specific embodiments, a chargeable species is a species that is charged to an cation at about neutral pH. It should be emphasized that not every charged cationic species on a polymer will be cationic at a pH near the pKₐ (acid dissociation constant) of the charged cationic species, but rather an equilibrium of cationic and non-cationic species will co-exist. “Chargeable monomeric units” described herein are used interchangeably with “chargeable monomeric residues”.

As used herein, “substantially non-charged” or “charge neutralized” includes a Zeta potential that is between ±10 to ±30 mV, and/or the presence of a first number (z) of chargeable species that are chargeable to a negative charge (e.g., acidic species that become anionic upon de-protonation) and a second number (0.5·z) of chargeable species that are chargeable to a positive charge (e.g., basic species that become cationic upon protonation).

As used herein, a “linking moiety” or a “linker” is a chemical bond or a multifunctional (e.g., bifunctional) residue which is used to link an RNAi agent, e.g., an oligonucleotide, and/or a targeting agent to the block co polymer. Linker moieties comprise any of a variety of compounds which can form an amide, ester, ether, thioether, carbamate, urea, amine or other linkage, e.g., linkages which are commonly used for immobilization of biomolecules in affinity chromatography. In some embodiments, the linking moiety comprises a cleavable bond, e.g. a bond that is unstable and/or is cleaved upon changes in certain intracellular parameters (e.g., pH or redox potential). In some embodiments, the linking moiety is non-cleavable. In certain embodiments, the linking moiety is attached to the RNAi agent or a targeting agent by one or more covalent bonds. In some embodiments, the linking moiety is attached to the pH-dependent membrane destabilizing polymer through one or more covalent bonds.

Hydrophobic species: “hydrophobic species” (used interchangeably herein with “hydrophobicity-enhancing moiety”), as used herein, is a moiety such as a substituent, residue or a group which, when covalently attached to a molecule, such as a monomer or a polymer, increases the molecule's hydrophobicity or serves as a hydrophobicity enhancing moiety. The term “hydrophobicity” is a term of art describing a physical property of a compound measured by the free energy of transfer of the compound between a non-polar solvent and water (Hydrophobicity regained. Karplus P. A., Protein Sci., 1997, 6: 1302-1307.) A compound's hydrophobicity can be measured by its logP value, the logarithm of a partition coefficient (P), which is defined as the ratio of concentrations of a compound in the two phases of a mixture of two immiscible solvents, e.g. octanol and water. Experimental methods of determination of hydrophobicity as well as methods of computer-assisted calculation of logP values are known to those skilled in the art. Hydrophobic species of the present invention include but are not limited to aliphatic, heteroaliphatic, aryl, and heteroaryl groups.

As used herein, a “hydrophobic core” comprises hydrophobic moieties. In certain instances, a “hydrophobic core” is substantially non-charged (e.g., the charge is substantially net neutral).

Without being bound by theory not expressly recited in the claims, a membrane destabilizing polymer can directly or indirectly elicit a change (e.g., a permeability change) in a cellular membrane structure (e.g., an endosomal membrane) so as to permit an agent (e.g., polynucleotide), in association with or independent of a micelle (or a constituent polymer thereof), to pass through such membrane structure—for example to enter a cell or to exit a cellular vesicle (e.g., an endosome). A membrane destabilizing polymer can be (but is not necessarily) a membrane disruptive polymer. A membrane disruptive polymer can directly or indirectly elicit lysis of a cellular vesicle or disruption of a cellular membrane (e.g., as observed for a substantial fraction of a population of cellular membranes).

Generally, membrane destabilizing or membrane disruptive properties of polymers or micelles can be assessed by various means. In one non-limiting approach, a change in a cellular membrane structure can be observed by assessment in assays that measure (directly or indirectly) release of an agent (e.g., polynucleotide) from cellular membranes (e.g., endosomal membranes)—for example, by determining the presence or absence of such agent, or an activity of such agent, in an environment external to such membrane. Another non-limiting approach involves measuring red blood cell lysis (hemolysis)—e.g., as a surrogate assay for a cellular membrane of interest. Such assays may be done at a single pH value or over a range of pH values.

As used herein, a “micelle” includes a particle comprising a core and a hydrophilic shell, wherein the core is held together at least partially, predominantly or substantially through hydrophobic interactions. In certain instances, as used herein, a “micelle” is a multi-component, nanoparticle comprising at least two domains, the inner domain or core, and the outer domain or shell. The core is at least partially, predominantly or substantially held together by hydrophobic interactions, and is present in the center of the micelle. As used herein, the “shell of a micelle” is defined as non-core portion of the micelle.

A “pH dependent membrane-destabilizing hydrophobe” is a group that is at least partially, predominantly, or substantially hydrophobic and is membrane destabilizing in a pH dependent manner. In certain instances, a pH dependent membrane destabilizing chargeable hydrophobe is a hydrophobic polymeric segment of a block copolymer and/or comprises a plurality of hydrophobic species; and comprises a plurality of anionic chargeable species. In some embodiments, the anionic chargeable species is anionic at about neutral pH. In further or alternative embodiments, the anionic chargeable species is non-charged at a lower, e.g., endosomal pH. In some embodiments, the membrane destabilizing chargeable hydrophobe comprises a plurality of cationic species. The pH dependent membrane-destabilizing chargeable hydrophobe comprises a non-peptidic and non-lipidic polymer backbone.

As used herein, normal physiological pH refers to the pH of the predominant fluids of the mammalian body such as blood, serum, the cytosol of normal cells, etc. In certain instances, normal physiologic pH is about neutral pH, including, e.g., a pH of about 7.2 to about 7.4. In some instances, about neutral pH is a pH of 6.6 to 7.6. As used herein, the terms neutral pH, physiologic and physiological pH are synonymous and interchangeable.

As used herein, a micelle is described as “stable” if the assembly does not disassociate or become destabilized in an aqueous solution representing physiological conditions, for example phosphate-buffered saline at pH 7.4. Micelle stability can be quantitatively defined by the critical micelle concentration (CMC), defined as the micelle concentration where instability occurs, as indicated by uptake of a hydrophobic probe molecule (e.g., the pyrene fluorescence assay) or changes in the size of the micelle (e.g., as determined by dynamic light scattering measurements). In certain instances, a stable micelle is one that has a hydrodynamic particle size that is within approximately 60%, 50%, 40%, 30%, 20%, or 10% of the hydrodynamic particle size of a micelle comprising the same block copolymers initially formed in an aqueous solution at a pH of 7.4 (e.g., a phosphate-buffered saline, pH 7.4). In some instances, a stable micelle is one that has a concentration of formation/assembly that is within about 60%, 50%, 40%, 30%, 20%, or 10% of the concentration of formation/assembly of a micelle comprising the same block copolymers initially in an aqueous solution at a pH of 7.4 (e.g., a phosphate-buffered saline, pH 7.4).

As used herein, a micelle is “destabilized” if it does not function in an identical, substantially similar or similar manner and/or possess identical, substantially similar or similar physical and/or chemical characteristics as would a stable micelle. Any “destabilization” of a micelle can be determined in any suitable manner. In one instance, a micelle is “destabilized” if it does not have a hydrodynamic particle size that is less than 5 times, 4 times, 3 times, 2 times, 1.8 times, 1.6 times, 1.5 times, 1.4 times, 1.3 times, 1.2 times, or 1.1 times the hydrodynamic particle size of a micelle comprising the same block copolymers and as formed in an aqueous solution at a pH of 7.4, or formed in human serum. In one instance, a micelle is “destabilized” if it does not have a concentration of assembly that is less than 5 times, 4 times, 3 times, 2 times, 1.8 times, 1.6 times, 1.5 times, 1.4 times, 1.3 times, 1.2 times, or 1.1 times the concentration of assembly of a micelle comprising the same block copolymers and as formed in an aqueous solution at a pH of 7.4, or formed in human serum.

Nanoparticle: As used herein, the term “nanoparticle” refers to any particle having a diameter of less than 1000 nanometers (nm). In general, the nanoparticles should have dimensions small enough to allow their uptake by eukaryotic cells. Typically the nanoparticles have a longest straight dimension (e.g., diameter) of 200 nm or less. In some embodiments, the nanoparticles have a diameter of 100 nm or less. Smaller nanoparticles, e.g. having diameters of about 10 nm to about 200 nm, about 20 nm to about 100 nm, about 10 nm to about 50 nm or 10 nm-30 nm, are used in some embodiments.

Oligonucleotide knockdown agent: as used herein, an “oligonucleotide knockdown agent” is an oligonucleotide species which can inhibit gene expression by targeting and binding an intracellular nucleic acid in a sequence-specific manner. Non-limiting examples of oligonucleotide knockdown agents include siRNA, miRNA, shRNA, dicer substrates, antisense oligonucleotides, decoy DNA or RNA, antigene oligonucleotides and any analogs and precursors thereof.

As used herein, the term “nucleotide,” in its broadest sense, refers to any compound and/or substance that is or can be incorporated into a polynucleotide (e.g., oligonucleotide) chain. In some embodiments, a nucleotide is a compound and/or substance that is or can be incorporated into a polynucleotide (e.g., oligonucleotide) chain via a phosphodiester linkage. In some embodiments, “nucleotide” refers to individual nucleic acid residues (e.g. nucleotides and/or nucleosides). In certain embodiments, “at least one nucleotide” refers to one or more nucleotides present; in various embodiments, the one or more nucleotides are discrete nucleotides, are non-covalently attached to one another, or are covalently attached to one another. As such, in certain instances, “at least one nucleotide” refers to one or more polynucleotide (e.g., oligonucleotide). In some instances, a polynucleotide is a polymer comprising at least two nucleotide monomeric units.

As used herein, the term “oligonucleotide” refers to a polymer comprising 7-200 nucleotide monomeric units. In some embodiments, “oligonucleotide” encompasses single and or/double stranded RNA as well as single and/or double-stranded DNA. Furthermore, the terms “nucleotide”, “nucleic acid,” “DNA,” “RNA,” and/or similar terms include nucleic acid analogs, i.e. analogs having a modified backbone, including but not limited to peptide nucleic acids (PNA), locked nucleic acids (LNA), phosphono-PNA, morpholino nucleic acids, or nucleic acids with modified phosphate groups (e.g., phosphorothioates, phosphonates, 5′-N-phosphoramidite linkages). Nucleotides can be purified from natural sources, produced using recombinant expression systems and optionally purified, chemically synthesized, etc. As used herein, a “nucleoside” is the term describing a compound comprising a monosaccharide and a base. The monosaccharide includes but is not limited to pentose and hexose monosaccharides. The monosaccharide also includes monosaccharide mimetics and monosaccharides modified by substituting hydroxyl groups with halogens, methoxy, hydrogen or amino groups, or by esterification of additional hydroxyl groups. In some embodiments, a nucleotide is or comprises a natural nucleoside phosphate (e.g. adenosine, thymidine, guanosine, cytidine, uridine, deoxyadenosine, deoxythymidine, deoxyguanosine, and deoxycytidine phosphate). In some embodiments, the base includes any bases occurring naturally in various nucleic acids as well as other modifications which mimic or resemble such naturally occurring bases. Nonlimiting examples of modified or derivatized bases include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl)uracil, 2-aminoadenine, pyrrolopyrimidine, and 2,6-diaminopurine. Nucleoside bases also include universal nucleobases such as difluorotolyl, nitroindolyl, nitropyrrolyl, or nitroimidazolyl. Nucleotides also include nucleotides which harbor a label or contain abasic, i.e. lacking a base, monomers. A nucleic acid sequence is presented in the 5′ to 3′ direction unless otherwise indicated. A nucleotide can bind to another nucleotide in a sequence-specific manner through hydrogen bonding via Watson-Crick base pairs. Such base pairs are said to be complementary to one another. An oligonucleotide can be single stranded, double-stranded or triple-stranded.

RNAi agent: As used herein, the term “RNAi agent” refers to an oligonucleotide which can mediate inhibition of gene expression through an RNAi mechanism and includes but is not limited to siRNA, microRNA (miRNA), short hairpin RNA (shRNA), asymmetrical interfering RNA (aiRNA), dicer substrate and the precursors thereof.

Short interfering RNA (siRNA): As used herein, the term “short interfering RNA” or “siRNA” refers to an RNAi agent comprising a nucleotide duplex that is approximately 15-50 base pairs in length and optionally further comprises zero to two single-stranded overhangs. One strand of the siRNA includes a portion that hybridizes with a target RNA in a complementary manner. In some embodiments, one or more mismatches between the siRNA and the targeted portion of the target RNA may exist. In some embodiments, siRNAs mediate inhibition of gene expression by causing degradation of target transcripts.

Short hairpin RNA (shRNA): Short hairpin RNA (shRNA) refers to an oligonucleotide having at least two complementary portions hybridized or capable of hybridizing with each other to form a double-stranded (duplex) structure and at least one single-stranded portion.

Dicer Substrate: a “dicer substrate” is a greater than approximately 25 base pair duplex RNA that is a substrate for the RNase III family member Dicer in cells. Dicer substrates are cleaved to produce approximately 21 base pair duplex small interfering RNAs (siRNAs) that evoke an RNA interference effect resulting in gene silencing by mRNA knockdown.

Therapeutic agent: As used herein, the phrase “therapeutic agent” refers to any agent that, when administered to a subject, organ, tissue, or cell has a therapeutic effect and/or elicits a desired biological and/or pharmacological effect, including but not limited to polynucleotides, oligonucleotides, RNAi agents, peptides and proteins.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition.

### Micelle Properties

Provided herein are micelles for intracellular delivery of diagnostic agents and/or therapeutic agents (e.g., oligonucleotides, peptides or the like). In some embodiments, such intracellular delivery is in vitro; in other embodiments, such intracellular delivery is in vivo. In some embodiments, the micelles provided herein are specifically designed for targeted delivery of a micellar payload at a desired site of therapeutic intervention in a subject. In some embodiments, a micelle, as described herein, has certain desired properties. For example, a micelle may be desired that is stable under certain circumstances (e.g., at neutral/physiologic pH), and less stable under other circumstances (e.g., at more acidic pH). Accordingly, the materials provided herein disclose certain parameters that contribute to such desired micellar properties.

In some embodiments, the micelles provided herein are stable under physiological conditions and have critical micellar concentrations that prevent undesired dissociation of the micelle. In further or alternative embodiments, the integrity of a micelle (e.g., in the physiological milieu) is also dependent on the composition of the block copolymers that comprise a micelle. Accordingly, provided herein are certain parameters (e.g., the number average molecular weight ratios for block copolymers in the shell block and the core block of micelles, number of charged moieties in the block copolymers, and the like) that are engineered to provide micelles suitable for efficient intracellular delivery of therapeutic agents with minimal toxicity and/or loss of micellar payload.

Accordingly, described herein are compositions that comprise a micelle and a polynucleotide associated with the micelle, the micelle comprising a plurality of block copolymers associating such that the micelle is stable in an aqueous medium at about neutral pH. Further, the micelles described herein have at least one of the following properties:
(i) the micelle comprising from about 10 to about 100 of the block copolymers per micelle,
(ii) a critical micelle concentration, CMC, ranging from about 0.2 μg/mL to about 20 μg/mL,
(iii) spontaneous micelle assembly in the absence of nucleic acid (iv) a particle size of about 5 nm to about 500 nm;
(v) a weight average molecular weight of about 0.5×10⁶ to about 3.6×10⁶ dalton.

In some embodiments, any micelle provided herein is characterized by having at least two of the aforementioned properties. In some embodiments, any micelle provided herein is characterized by having at least three of the aforementioned properties. In some embodiments, any micelle provided herein is characterized by having all of the aforementioned properties. In some embodiments, a micelle described herein is stable to high ionic strength of the surrounding media (e.g. 0.5M NaCl); and/or the micelle has an increasing instability as the concentration of organic solvent increases, such organic solvents including, but not limited to dimethylformamide (DMF), dimethylsulfoxide (DMS), and dioxane.

### Composition of Micelles

Micelles provided herein comprise a plurality of polymers per micelle. In some embodiments, the polymers are copolymers. In further embodiments, the copolymer is a block copolymer. The block copolymer is a monoblock polymer or a multiblock polymer (e.g., a diblock polymer). The term “copolymer”, as used herein, signifies that the polymer is the result of polymerization of two or more different monomers. A “monoblock polymer” is a synthetic product of a single polymerization step. The term monoblock polymer includes a copolymer (i.e. a product of polymerization of more than one type of monomers) and a homopolymer (i.e. a product of polymerization of a single type of monomers). A “block” copolymer refers to a structure comprising one or more sub-combination of constitutional or monomeric units. In some embodiments, monomer residues found in the polymer are further modified in order to arrive at the constitutional units. In some embodiments, a block copolymer described herein comprises non-lipidic constitutional or monomeric units. In some embodiments, the block copolymer is a diblock copolymer. A diblock copolymer comprises two blocks; a schematic generalization of such a polymer is represented by the following: [AₐB_{b}C_{c} . . . ]ₘ-[XₓY_{y}Z_{z} . . . ]ₙ, wherein each letter stands for a monomeric or monomeric unit, and wherein each subscript to a monomeric unit represents the mole fraction of that unit in the particular block, the three dots indicate that there may be more (there may also be fewer) monomeric units in each block and m and n indicate the molecular weight of each block in the diblock copolymer. As suggested by the schematic, in some instances, the number and the nature of each monomeric unit is separately controlled for each block. The schematic is not meant and should not be construed to infer any relationship whatsoever between the number of monomeric units or the number of different types of monomeric units in each of the blocks. Nor is the schematic meant to describe any particular number or arrangement of the monomeric units within a particular block. In each block the monomeric units may be disposed in a purely random, an alternating random, a regular alternating, a regular block or a random block configuration unless expressly stated to be otherwise. A purely random configuration, for example, may have the non-limiting form: x-x-y-z-x-y-y-z-y-z-z-z . . . . A non-limiting, exemplary alternating random configuration may have the non-limiting form: x-y-x-z-y-x-y-z-y-x-z . . . , and an exemplary regular alternating configuration may have the non-limiting form: x-y-z-x-y-z-x-y-z . . . . An exemplary regular block configuration may have the following non-limiting configuration: . . . x-x-x-y-y-y-z-z-z-x-x-x . . . , while an exemplary random block configuration may have the non-limiting configuration: . . . x-x-x-z-z-x-x-y-y-y-y-z-z-z-x-x-z-z-z- . . . . In a gradient polymer, the content of one or more monomeric units increases or decreases in a gradient manner from the alpha end of the polymer to the omega end. In none of the preceding generic examples is the particular juxtaposition of individual monomeric units or blocks or the number of monomeric units in a block or the number of blocks meant nor should they be construed as in any manner bearing on or limiting the actual structure of block copolymers forming the micelles of this invention.

As used herein, the brackets enclosing the monomeric units are not meant and are not to be construed to mean that the monomeric units themselves form blocks. That is, the monomeric units within the square brackets may combine in any manner with the other monomeric units within the block, i.e., purely random, alternating random, regular alternating, regular block or random block configurations. The copolymers described herein are, optionally, alternate, gradient or random copolymers. In some instances, the copolymer consists essentially of a random copolymer.

In some embodiments, a micelle described herein comprises from about 10 to about 500 block copolymers per micelle. In some embodiments, a micelle described herein comprises from about 10 to about 250 block copolymers per micelle. In some embodiments, a micelle described herein comprises from about 10 to about 100 block copolymers per micelle. In some embodiments, a micelle described herein comprises from about 30 to about 50 block copolymers per micelle.

### Micelle Formation and Stability

In some embodiments, a micelle provided herein is formed by spontaneous self association of block copolymers to form organized assemblies (e.g., micelles) upon dilution from a water-miscible solvent (such as but not limited to ethanol) to aqueous solvents (for example phosphate-buffered saline, pH 7.4). In some embodiments, micelle formation occurs by directly dissolving a dried form of the polymer in an aqueous solvent. In some embodiments, spontaneous micelle formation occurs in the absence of polynucleotides or oligonucleotides.

In some embodiments, a micelle described herein is stable upon dilution from a water-miscible solvent (such as but not limited to ethanol) to aqueous solvents to a pH of about 7.4 to about 5.5. In some embodiments, a micelle described herein is stable upon dilution from a water-miscible solvent (such as but not limited to ethanol) to aqueous solvents to a pH of about 7.4 to about 6.8. In some embodiments, a micelle described herein is stable upon dilution from a water-miscible solvent (such as but not limited to ethanol) to aqueous solvents to a pH of about 7.4, about 7.2, about 7.0, about 6.8, about 6.4, about 6.2, about 6.0 or about 5.8. In some embodiments, a micelle provided herein is stable in an aqueous medium. In certain embodiments, a micelle provided herein is stable in an aqueous medium at a selected pH, e.g., about physiological pH (e.g., the pH of circulating human plasma). In specific embodiments, a micelle provided herein is stable at about a neutral pH (e.g., at a pH of about 7.4) in an aqueous medium. In specific embodiments, the aqueous medium is animal (e.g., human) serum or animal (e.g., human) plasma. It is to be understood that stability of the micelle is not limited to designated pH, but that it is stable at pH values that include, at a minimum, the designated pH. In specific embodiments, a micelle described herein is substantially less stable at an acidic pH than at a pH that is about neutral. In more specific embodiments, a micelle described herein is substantially less stable at a pH of about 5.8 than at a pH of about 7.4.

In specific embodiments, at about neutral pH, a micelle described herein is stable at a concentration of about 10 μg/mL, about 50 μg/mL, about 100 μg/mL, about 200 μg/mL, or about 250 μg/mL.

In some embodiments, the micelles are stable to dilution in an aqueous solution. In specific embodiments, the micelles are stable to dilution at physiologic pH (e.g., pH of circulating blood in a human) with a critical stability concentration (e.g., a critical micelle concentration (CMC)) of about 100 μg/mL to about 0.1 μg/mL, about 100 μg/mL to about 1 μg/mL, about 50 μg/mL to about 1 μg/mL, about 50 to about 10 μg/mL. In some embodiments, the CMC of a micelle at physiologic pH is less than 100 μg/mL, less than 50 μg/mL, less than 10 μg/mL, less than 5 μg/mL, or less than 2 μg/mL. As used herein, “destabilization of a micelle” means that the polymeric chains forming a micelle at least partially disaggregate, structurally alter (e.g., expand in size and/or change shape), and/or may form amorphous supramolecular structures (e.g., non-micellic supramolecular structures). The terms critical stability concentration (CSC), critical micelle concentration (CMC), and critical assembly concentration (CAC) are used interchangeably herein. In some embodiments, a micelle described herein is stable to dilution which constitutes the critical stability concentration or the critical micelle concentration (CMC).

In some embodiments, the critical stability concentration or the CMC of any micelle described herein is from about 100 μg/mL to about 0.1 μg/mL at about neutral pH. In some embodiments the CMC of a micelle described herein is from about 80 μg/mL to about 0.2 μg/mL, from about 60 μg/mL to about 0.2 μg/mL, from about 40 μg/mL to about 0.2 μg/mL, from about 20 μg/mL to about 0.2 μg/mL, or from about 10 μg/mL to about 0.2 μg/mL at about neutral pH. In some embodiments, the CMC of a micelle described herein is about 100 μg/mL, about 90 μg/mL, about 80 μg/mL, about 70 μg/mL, about 60 μg/mL, about 50 μg/mL, about 40 μg/mL, about 30 μg/mL, about 20 μg/mL, about 10 μg/mL, about 5 μg/mL, about 1 μg/mL, about 0.5 μg/mL, or about 0.2 μg/mL at about neutral pH.

In some embodiments, the critical micelle concentration or the CMC of any micelle described herein at endosomolytic pH (e.g. pH of about 5) is about 20-fold higher than the CMC of the micelle at about neutral pH (e.g., pH of about 7.4). In certain embodiments, the critical micelle concentration or the CMC of any micelle described herein at endosomolytic pH (e.g. pH of about 5) is about 10-fold higher than the CMC of the micelle at about neutral pH (e.g., pH of about 7.4). In some embodiments, the critical stability concentration or the CMC of any micelle described herein at endosomolytic pH (e.g. pH of about 5) is about 5-fold higher, or about 2-fold higher than the CMC of the micelle at physiological pH (e.g., pH of about 7.4).

In some embodiments, the critical micelle concentration or the CMC of any micelle described herein at endosomolytic pH (e.g. pH of about 5) is from about 100 μg/mL to about 0.5 μg/mL, from about 80 μg/mL to about 1 μg/mL, from about 60 μg/mL to about 1 μg/mL, from about 40 μg/mL to about 1 μg/mL, from about 20 μg/mL to about 1 μg/mL, or from about 10 μg/mL to about 1 μg/mL. In some embodiments, the CMC of a micelle described herein is about 100 μg/mL, about 90 μg/mL, about 80 μg/mL, about 70 μg/mL, about 60 μg/mL, about 50 μg/mL, about 40 μg/mL, about 30 μg/mL, about 20 μg/mL, about 10 μg/mL, about 5 μg/mL, about 1 μg/mL, or about 0.5 μg/mL, at about endosomolytic pH.

### Particle Size

In certain embodiments, the micelle is a nanoparticle. In specific embodiments, the micelle is a true micelle. In yet further embodiments, the micelle is a nanoparticle or micelle with a mean hydrodynamic particle size in the absence of conjugation to a bioactive agent of approximately 10 nm to about 200 nm, about 10 nm to about 100 nm, or about 30-80 nm. Particle size can be determined in any manner, including, but not limited to, by gel permeation chromatography (GPC), dynamic light scattering (DLS), electron microscopy techniques (e.g., TEM), and other methods.

In specific embodiments, a micelle described herein comprises a block copolymer that is associated (e.g. ionically and/or covalently) to a bioactive agent (e.g., a polynucleotide (e.g. siRNA), a diagnostic agent and/or a targeting agent (e.g., an antibody)) and has a particle size of not more than about 500 nm, not more than about 450 nm, not more than about 400 nm, not more than about 350 nm, not more than about 300 nm, or not more than about 250 nm, not more than about 200 nm, not more than about 150 nm, not more than about 100 nm, or not more than about 50 nm.

### Polynucleotide Loading

In some embodiments, a micelle described herein is associated (e.g., ionically and/or covalently) with from 1 to about 10,000 polynucleotides. In some embodiments, a micelle described herein is associated with about 4 to about 5000, about 10 to about 4000, about 15 to about 3000, or about 30 to about 2500 polynucleotides. In some embodiments, the charge ratio of a micelle to a polynucleotide is from about 5:1 to about 1:1. In some embodiments, the charge ratio of a micelle to a polynucleotide is about 4:1, about 3:1, about 2:1 or about 1:1.

### Polymer Architecture and Properties

In certain embodiments, a block copolymer described herein comprises a hydrophilic block and a hydrophobic block. In some embodiments, at least one of such blocks is a gradient polymer block. In further embodiments, the block copolymer utilized herein is optionally substituted with a gradient polymer (i.e., the polymer utilized in the micelle is a gradient polymer having a hydrophobic block and a hydrophilic block).

### Hydrophilic Block

In certain embodiments, the hydrophilic block is a shell block and is e.g., a non-charged, cationic, polycationic, anionic, polyanionic, or zwitterionic block. In certain embodiments, the hydrophilic block is neutral (non-charged). In specific embodiments, the hydrophilic block comprises a net positive charge. In specific embodiments, the hydrophilic block comprises a net negative charge. In specific embodiments, the hydrophilic block comprises a net neutral charge.

In some embodiments, a hydrophilic block is a homopolymer block comprising a single monomer. In other embodiments, a hydrophilic block comprises a plurality of one or more hydrophilic monomeric units (e.g., one or more of DMAEMA, PEGMA, HPMA, oligoethyleneglycol acrylate, NIPAAM, or the like). In certain embodiments, the hydrophilic monomeric units comprise hydrophilic groups (e.g., hydroxyl groups, thiol groups, PEG groups or other polyoxylated alkyl groups, or the like, or a combination thereof). In some embodiments, the hydrophilic monomeric units are substantially non-chargeable, e.g., meaning that the hydrophilic monomeric units are substantially non-charged at physiological pH (e.g., pH about neutral such as 7.2-7.4). In some embodiments, the block copolymer comprises more than 5, more than 10, more than 20, more than 50 or more than 100 hydrophilic groups or species.

In certain embodiments, block copolymers described herein each have (1) a neutral or non-charged (e.g., substantially non-charged) hydrophilic block; and (2) a hydrophobic block (e.g., a core block) forming the hydrophobic core of the micelle which is stabilized through hydrophobic interactions of the core-forming polymeric segments. In certain embodiments, the neutral or non-charged hydrophilic block comprises a plurality of neutral monomeric residues such as PEGMA or HPMA.

In certain embodiments, block copolymers described herein each have (1) a cationic or polycationic charged hydrophilic block; and (2) a hydrophobic block (e.g., a core block) forming the hydrophobic core of the micelle which is stabilized through hydrophobic interactions of the core-forming polymeric segments. In certain embodiments, the hydrophilic block comprises a plurality of cationic monomeric residues such as DMAEMA. In some of such embodiments, a polynucleotide is in ionic association with the cationic species in a hydrophilic block.

In certain embodiments, block copolymers described herein each have (1) an anionic or polyanionic charged hydrophilic block; and (2) a hydrophobic block (e.g., a core block) forming the hydrophobic core of the micelle which is stabilized through hydrophobic interactions of the core-forming polymeric segments. In certain embodiments, the anionic or polyanionic charged hydrophilic block comprises a plurality of anionic monomeric residues such as maleic anhydride or acrylic acid.

In certain embodiments, block copolymers described herein each have (1) a zwitterionic or polyzwitterionic charged hydrophilic block; and (2) a hydrophobic block (e.g., a core block) forming the hydrophobic core of the micelle which is stabilized through hydrophobic interactions of the core-forming polymeric segments.

### Hydrophobic Block

In certain embodiments, a hydrophobic block of any block copolymer described herein comprises a plurality of hydrophobic groups, moieties, monomeric units, species, or the like. In certain embodiments, a hydrophobic block of any block copolymer described herein comprises a plurality of hydrophobic groups, moieties, monomeric units, species, or the like and a plurality of chargeable constitutional units or monomeric units.

In certain embodiments, a block copolymer comprises a hydrophobic block comprising a first and a second constitutional unit. In certain embodiments, the first constitutional unit comprises an anionic species upon deprotonation. In certain embodiments, the first constitutional unit is non-charged at an acidic pH (e.g., an endosomal pH, a pH below about 6.5, a pH below about 6.0, a pH below about 5.8, a pH below about 5.7, or the like). In some embodiments, the first constitutional unit is as described herein and the second constitutional unit is a cationic species upon protonation. In specific embodiments, the pKa of the second constitutional unit is about 6 to about 10, about 6.5 to about 9, about 6.5 to about 8, about 6.5 to about 7.5, or any other suitable pKa.

In some embodiments, the hydrophobic block of any block copolymer described herein further comprises hydrophobic groups, moieties, monomeric units, species, or the like. In some embodiments, the hydrophobic monomeric unit comprises a hydrophobic group such as but not limited to an alkyl group, a heteroalkyl group, an aryl group, or a heteroaryl group. In some embodiments, a block copolymer comprises a hydrophobic group that is attached to the polymer backbone and shields a vicinal chargeable constitutional unit (e.g. an anionic moiety (e.g., a carboxylic acid group)) thereby reducing or preventing dissociation of a micelle. In some embodiments, a hydrophobic block of a block copolymer comprises more than 5, more than 10, more than 20, more than 50 or more than 100 hydrophobic groups or species. In some embodiments, the hydrophobic species are present on the anionic chargeable monomeric units. In some embodiments, the ratio of the hydrophobic monomeric units to the monomeric units comprising a constitutional unit that is chargeable to an anion is between about 1:6 and about 1:1, about 1:5 and about 1:1, about 1:4 and about 1:1, about 1:3 and about 1:1, about 1:2 and about 1:1 at about a neutral pH.

In some embodiments, the hydrophobic monomeric unit is, by way of non-limiting example, a butyl methacrylate, butyl acrylate, styrene, or the like. In specific embodiments, hydrophobic monomeric unit useful herein is a monomeric unit derived from (C₂-C₈)alkyl ester of (C₂-C₈)alkylacrylic acid.

In more specific embodiments, the hydrophobic block of a block copolymer described herein comprises a plurality of cationic monomeric units and a plurality of anionic monomeric units. In still more specific embodiments, the hydrophobic block comprises a substantially similar number of cationic and anionic species (i.e., the hydrophobic block and/or core of the micelle are substantially net neutral). In some embodiments, the presence of a substantially similar number of cationic and anionic species in the hydrophobic block of a block copolymer provides a hydrophobic block and/or core of the micelle that is substantially net neutral at about neutral pH.

### Anionic Constitutional Units

In some embodiments, a block copolymer described herein comprises a plurality of anionic constitutional units that are anionic at physiological pH. In some embodiments, anionic constitutional units comprise protonatable anionic species. In certain embodiments, a block copolymer described herein comprises a plurality of anionic constitutional units and each anionic constitutional unit is a residue of a non-charged Brønsted acid monomer (i.e., the constitutional unit is a conjugate base of a Brønsted acid). In various embodiments described herein, constitutional units, that are anionic or negatively charged at physiological pH (including, e.g., certain hydrophilic constitutional units) described herein comprise one or more acid group or conjugate base thereof. Non-limiting examples of anionic constitutional units include monomeric residues comprising carboxylic acid, sulfonamide, boronic acid, sulfonic acid, sulfinic acid, sulfuric acid, phosphoric acid, phosphinic acid or the like and or combinations thereof. In some embodiments, constitutional units that are anionic or negatively charged at normal physiological pH that are utilized herein include, by way of non-limiting example, monomeric residues of acrylic acid, C₂-C₈ alkylacrylic acid monomers (e.g., methyl acrylic acid, ethyl acrylic acid, propyl acrylic acid, butyl acrylic acid, etc.), or the like.

When the pH of a physiological fluid is at about the pKₐ of an anionic species, there will exist an equilibrium distribution of chargeable species in both forms. In the case of an anionic species, about 50% of the population will be anionic and about 50% will be non-charged when the pH is at the pKₐ of the anionic species. The further the pH is from the pKₐ of the chargeable species, there will be a corresponding shift in this equilibrium such that at higher pH values, the anionic form will predominate and at lower pH values, the uncharged form will predominate. The embodiments described herein include the form of the block copolymers at any pH value.

In some embodiments, constitutional units that are anionic at normal physiological pH comprise carboxylic acids such as, without limitation, monomeric residues of 2-propyl acrylic acid (i.e., the constitutional unit derived from it, 2-propylpropionic acid, —CH₂C((CH₂)₂CH₃)(COOH)—(PAA)), although any organic or inorganic acid that can be present, either as a protected species, e.g., an ester, or as the free acid, in the selected polymerization process is also within the contemplation of this invention. Anionic monomeric residues or constitutional units described herein comprise a species charged or chargeable to an anion, including a protonatable anionic species. In certain instances, anionic monomeric residues can be anionic at about neutral pH.

Monomers such as maleic-anhydride, (Scott M. Henry, Mohamed E. H. El-Sayed, Christopher M. Pirie, Allan S. Hoffman, and Patrick S. Stayton “pH-Responsive Poly(styrene-alt-maleic anhydride) Alkylamide Copolymers for Intracellular Drug Delivery” Biomacromolecules 7:2407-2414, 2006) may also be used for introduction of anionic species into the hydrophobic block. In such embodiments, the negatively charged constitutional unit is derived from a maleic anhydride monomeric residue.

### Cationic Constitutional Units

In some embodiments, a block copolymer described herein comprises a plurality of cationic constitutional units that are cationic or positively charged at physiological pH. In some embodiments, cationic constitutional units comprise deprotonatable cationic species. In certain embodiments, a block copolymer described herein comprises a plurality of cationic constitutional units and each cationic constitutional unit is a residue of a non-charged Brønsted base monomer (i.e., the constitutional unit is a conjugate acid of a Brønsted base). Non-limiting examples of Brønsted base monomers include monomers that comprise dialkylamino groups. In some embodiments, a cationic constitutional unit comprises an acyclic amine, acyclic imine, cyclic amine, cyclic imine, amino groups, alkylamino groups, guanidine groups, imidazolyl groups, pyridyl groups, triazolyl groups or the like or combinations thereof. In some embodiments, constitutional units that are cationic at normal physiological pH that are utilized herein include, by way of non-limiting example, monomeric residues of dialkylaminoalkylmethacrylates (e.g., DMAEMA).

When the pH of a physiological fluid is at about the pKₐ of a cationic species, there will exist an equilibrium distribution of chargeable species in both forms. The further the pH is from the pKₐ of the chargeable species, there will be a corresponding shift in this equilibrium such that at lower pH values, the cationic form will predominate and at higher pH values, the uncharged form will predominate. The embodiments described herein include the form of the block copolymers at any pH value.

### Neutral and Zwitterionic Constitutional Units

In various embodiments described herein, constitutional units that are neutral at physiologic pH comprise one or more hydrophilic groups, e.g., hydroxy, polyoxylated alkyl, polyethylene glycol, polypropylene glycol, thiol, or the like. In some embodiments, hydrophilic constitutional units that are neutral at normal physiological pH that are utilized herein include, by way of non-limiting example, monomeric residues of PEGylated acrylic acid, PEGylated methacrylic acid, hydroxyalkylacrylic acid, hydroxyalkylalkacrylic acid (e.g., HPMA), or the like.

In various embodiments described herein, constitutional units that are zwitterionic at physiologic pH comprise an anionic or negatively charged group at physiologic pH and a cationic or positively charged group at physiologic pH. In some embodiments, hydrophilic constitutional units that are zwitterionic at normal physiological pH that are utilized herein include, by way of non-limiting example, monomeric residues of comprising a phosphate group and an ammonium group at physiologic pH, such as set forth in U.S. Pat. No. 7,300,990, which is hereby incorporated herein for such disclosure, or the like.

### Composition of Block Copolymers

In certain embodiments, the first constitutional unit is an anionic species upon deprotonation, the second constitutional unit is a cationic species upon protonation, and the ratio of the anionic species to the cationic species is between about 1:10 and about 10:1, about 1:6 and about 6:1, about 1:4 and about 4:1, about 1:2 and about 2:1, about 1:2 and 3:2, or about 1:1 at about a neutral pH. In some embodiments, the ratio of the first chargeable constitutional unit to the second chargeable constitutional unit is about 1:10 and about 10:1, about 1:6 and about 6:1, about 1:4 and about 4:1, about 1:2 and about 2:1, about 1:2 and 3:2, or about 1:1.

In some embodiments, the constitutional, groups, or monomeric units that are chargeable to anionic species, groups, or monomeric units present in the block copolymers are species, groups, or monomeric units that are at least 50%, at least 60%, at least 70%, at least 80%, at least 85%, or at least 95% negatively charged at about neutral pH (e.g., at a pH of about 7.4). In specific embodiments, these chargeable species, groups, or monomeric units are charged by loss of an H⁺, to an anionic species at about neutral pH. In further or alternative embodiments, the chargeable species, groups, or monomeric units that are chargeable to anionic species, groups, or monomeric units present in the polymer are species, groups, or monomeric units that are at least 20%, at least 30%, at least 40%, at least 50%, at least 60%, at least 70%, at least 80%, at least 85%, or at least 95% neutral or non-charged at a slightly acidic pH (e.g., a pH of about 6.5, or less; about 6.2, or less; about 6, or less; about 5.9, or less; about 5.8, or less; about 5.7, or less; about 5.6, or less, about 5.5, or less, about 5.0, or less; or about endosomal pH).

In specific embodiments of the block copolymers described herein, each constitutional unit is present on a different monomeric unit. In some embodiments, a first monomeric unit comprises the first chargeable species. In further or alternative embodiments, a second monomeric unit comprises the second chargeable species. In further or alternative embodiments, a third monomeric unit comprises a third chargeable species.

### Exemplary Structures

In certain embodiments, the block copolymer (e.g., membrane destabilizing block copolymer) has the chemical Formula I:

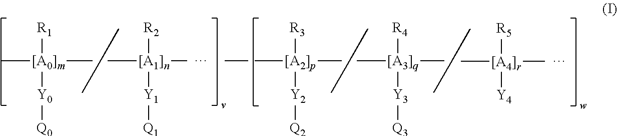

In some embodiments:
A₀, A₁, A₂, A₃ and A₄ are selected from the group consisting of —C—, —C—, —C—, —C(O)(C)ₐC(O)O—, —O(C)ₐC(O)— and —O(C)_{b}O—; wherein,
   a is 1-4;
   b is 2-4;
Y₄ is selected from the group consisting of hydrogen, (1C-10C)alkyl, (3C-6C)cycloalkyl, O—(1C-10C)alkyl, —C(O)O(1C-10C)alkyl, C(O)NR₆(1C-10C), (4C-10C)heteroaryl and (6C-10C)aryl, any of which is optionally substituted with one or more fluorine groups;
Y₀, Y₁ and Y₂ are independently selected from the group consisting of a covalent bond, (1C-10C)alkyl-, —C(O)O(2C-10C) alkyl-, —OC(O)(1C-10C) alkyl-, —O(2C-10C)alkyl- and —S(2C-10C)alkyl-, —C(O)NR₆(2C-10C) alkyl-, -(4C-10C)heteroaryl- and -(6C-10C)aryl-;
Y₃ is selected from the group consisting of a covalent bond, -(1C-10C)alkyl-, -(4C-10C)heteroaryl- and -(6C-10C)aryl-; wherein
   tetravalent carbon atoms of A₁-A₄ that are not fully substituted with R₁-R₅ and
   Y₀-Y₄ are completed with an appropriate number of hydrogen atoms;
R₁, R₂, R₃, R₄, R₅, and R₆ are independently selected from the group consisting of hydrogen, —CN, alkyl, alkynyl, heteroalkyl, cycloalkyl, heterocycloalkyl, aryl and heteroaryl, any of which may be optionally substituted with one or more fluorine atoms;
Q₀ is a residue selected from the group consisting of residues which are hydrophilic at physiologic pH, and are at least partially positively charged at physiologic pH (e.g., amino, alkylamino, ammonium, alkylammonium, guanidine, imidazolyl, pyridyl, or the like); at least partially negatively charged at physiologic pH but undergo protonation at lower pH (e.g., carboxyl, sulfonamide, boronate, phosphonate, phosphate, or the like); substantially neutral (or non-charged) at physiologic pH (e.g., hydroxy, polyoxylated alkyl, polyethylene glycol, polypropylene glycol, thiol, or the like); at least partially zwitterionic at physiologic pH (e.g., a monomeric residue comprising a phosphate group and an ammonium group at physiologic pH); conjugatable or functionalizable residues (e.g. residues that comprise a reactive group, e.g., azide, alkyne, succinimide ester, tetrafluorophenyl ester, pentafluorophenyl ester, p-nitrophenyl ester, pyridyl disulfide, or the like); or hydrogen;

Q₁ is a residue which is hydrophilic at physiologic pH, and is at least partially positively charged at physiologic pH (e.g., amino, alkylamino, ammonium, alkylammonium, guanidine, imidazolyl, pyridyl, or the like); at least partially negatively charged at physiologic pH but undergoes protonation at lower pH (e.g., carboxyl, sulfonamide, boronate, phosphonate, phosphate, or the like); substantially neutral at physiologic pH (e.g., hydroxy, polyoxylated alkyl, polyethylene glycol, polypropylene glycol, thiol, or the like); or at least partially zwitterionic at physiologic pH (e.g., comprising a phosphate group and an ammonium group at physiologic pH);
Q₂ is a residue which is positively charged at physiologic pH, including but not limited to amino, alkylamino, ammonium, alkylammonium, guanidine, imidazolyl, and pyridyl;
Q₃ is a residue which is negatively charged at physiologic pH, but undergoes protonation at lower pH, including but not limited to carboxyl, sulfonamide, boronate, phosphonate, and phosphate;
m is about 0 to less than 1.0 (e.g., 0 to about 0.49);
n is greater than 0 to about 1.0 (e.g., about 0.51 to about 1.0); wherein
   m+n=1
p is about 0.1 to about 0.9 (e.g., about 0.2 to about 0.5);
q is about 0.1 to about 0.9 (e.g., about 0.2 to about 0.5); wherein:
r is 0 to about 0.8 (e.g., 0 to about 0.6); wherein
   p+q+r=1
v is from about 1 to about 25 kDa, or about 5 to about 25 kDa; and,
w is from about 1 to about 50 kDa, or about 5 to about 50 kDa.

In some embodiments, the number or ratio of monomeric residues represented by p and q are within about 30% of each other, about 20% of each other, about 10% of each other, or the like. In specific embodiments, p is substantially the same as q. In certain embodiments, at least partially charged generally includes more than a trace amount of charged species, including, e.g., at least 20% of the residues are charged, at least 30% of the residues are charged, at least 40% of the residues are charged, at least 50% of the residues are charged, at least 60% of the residues are charged, at least 70% of the residues are charged, or the like.

In certain embodiments, m is 0 and Q₁ is a residue which is hydrophilic and substantially neutral (or non-charged) at physiologic pH. In some embodiments, substantially non-charged includes, e.g., less than 5% are charged, less than 3% are charged, less than 1% are charged, or the like. In certain embodiments, m is 0 and Q₁ is a residue which is hydrophilic and at least partially cationic at physiologic pH. In certain embodiments, m is 0 and Q₁ is a residue which is hydrophilic and at least partially anionic at physiologic pH. In certain embodiments, m is >0 and n is >0 and one of and Q₀ or Q₁ is a residue which is hydrophilic and at least partially cationic at physiologic pH and the other of Q₀ or Q₁ is a residue which is hydrophilic and is substantially neutral at physiologic pH. In certain embodiments, m is >0 and n is >0 and one of and Q₀ or Q₁ is a residue which is hydrophilic and at least partially anionic at physiologic pH and the other of Q₀ or Q₁ is a residue which is hydrophilic and is substantially neutral at physiologic pH. In certain embodiments, m is >0 and n is >0 and Q₁ is a residue which is hydrophilic and at least partially cationic at physiologic pH and Q₀ is a residue which is a conjugatable or functionalizable residue. In certain embodiments, m is >0 and n is >0 and Q₁ is a residue which is hydrophilic and substantially neutral at physiologic pH and Q₀ is a residue which is a conjugatable or functionalizable residue.

In certain embodiments, a micelle described herein comprises a block copolymer of Formula II:

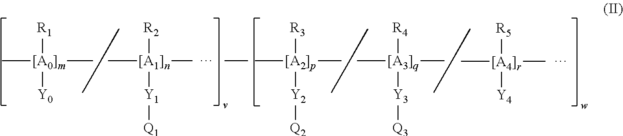

In some embodiments:
A₀, A₁, A₂, A₃ and A₄ are selected from the group consisting of —C—C—, —C(O)(C)ₐC(O)O—, —O(C)ₐC(O)— and —O(C)_{b}O—; wherein,
   a is 1-4;
   b is 2-4;
Y₀ and Y₄ are independently selected from the group consisting of hydrogen, (1C-10C)alkyl, (3C-6C)cycloalkyl, O—(1C-10C)alkyl, —C(O)O(1C-10C)alkyl, C(O)NR₆(1C-10C), (4C-10C)heteroaryl and (C6-C10)aryl, any of which is optionally substituted with one or more fluorine groups;
Y₁ and Y₂ are independently selected from the group consisting of a covalent bond, (1C-10C)alkyl-, —C(O)O(2C-10C)alkyl-, —OC(O)(1C-10C)alkyl-, —O(2C-10C)alkyl- and —S(2C-10C)alkyl-, —C(O)NR₆(2C-10C)alkyl-, -(4C-10C)heteroaryl- and -(6C-10C)aryl-;
Y₃ is selected from the group consisting of a covalent bond, (1C-10C)alkyl, -(4C-10C)heteroaryl- and (6C-10C)aryl; wherein
   tetravalent carbon atoms of A₁-A₄ that are not fully substituted with R₁-R₅ and Y₀-Y₄ are completed with an appropriate number of hydrogen atoms;
R₁, R₂, R₃, R₄, R₅, and R₆ are independently selected from the group consisting of hydrogen, —CN, alkyl, alkynyl, heteroalkyl, cycloalkyl, heterocycloalkyl, aryl and heteroaryl, any of which may be optionally substituted with one or more fluorine atoms;
Q₁ and Q₂ are residues which are positively charged at physiologic pH, including but not limited to amino, alkylamino, ammonium, alkylammonium, guanidine, imidazolyl, and pyridyl.
Q₃ is a residue which is negatively charged at physiologic pH, but undergoes protonation at lower pH, including but not limited to carboxyl, sulfonamide, boronate, phosphonate, and phosphate.
m is 0 to about 0.49;
n is about 0.51 to about 1.0; wherein
   m+n=1
p is about 0.2 to about 0.5;
q is about 0.2 to about 0.5; wherein:
   p is substantially the same as q;
r is 0 to about 0.6; wherein
   p+q+r=1
v is from about 1 to about 25 kDa, or about 5 to about 25 kDa; and,
w is from about 1 to about 50 kDa, or about 5 to about 50 kDa.

In certain embodiments, a micelle described herein comprises a block copolymer (e.g., at normal physiological pH) of Formula III:

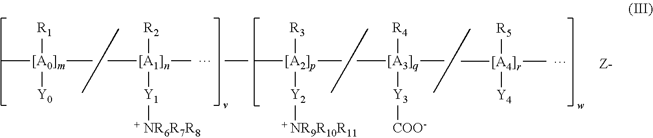

In certain embodiments, A₀, A₁, A₂, A₃, and A₄, substituted as indicated comprise the constitutional units (used interchangeably herein with “monomeric units” and “monomeric residues”) of the polymer of Formula III. In specific embodiments, the monomeric units of constituting the A groups of Formula III are polymerizably compatible under appropriate conditions. In certain instances, an ethylenic backbone or constitutional unit, —(C—C—)ₘ— polymer, wherein each C is di-substituted with H and/or any other suitable group, is polymerized using monomers containing a carbon-carbon double bond, >C═C<. In certain embodiments, each A group (e.g., each of A₀, A₁, A₂, A₃, and A₄) may be (i.e., independently selected from) —C≡C— (i.e., an ethylenic monomeric unit or polymer backbone), —C(O)(C)ₙC(O)O— (i.e., a polyanhydride monomeric unit or polymer backbone), —O(C)ₙC(O)— (i.e., a polyester monomeric unit or polymer backbone), —O(C)_{b}O— (i.e., a polyalkylene glycol monomeric unit or polymer backbone), or the like (wherein each C is di-substituted with H and/or any other suitable group such as described herein, including R₁₂ and/or R₁₃ as described above). In specific embodiments, the term “a” is an integer from 1 to 4, and “b” is an integer from 2 to 4. In certain instances, each “Y” and “R” group attached to the backbone of Formula III (i.e., any one of Y₀, Y₁, Y₂, Y₃, Y₄, R₁, R₂, R₃, R₄, R₅) is bonded to any “C” (including any (C)ₐ or (C)_{b}) of the specific monomeric unit. In specific embodiments, both the Y and R of a specific monomeric unit is attached to the same “C”. In certain specific embodiments, both the Y and R of a specific monomeric unit is attached to the same “C”, the “C” being alpha to the carbonyl group of the monomeric unit, if present.

In specific embodiments, R₁-R₁₁ are independently selected from hydrogen, alkyl (e.g., 1C-5C alkyl), cycloalkyl (e.g., 3C-6C cycloalkyl), or phenyl, wherein any of R₁-R₁₁ is optionally substituted with one or more fluorine, cycloalkyl, or phenyl, which may optionally be further substituted with one or more alkyl group.

In certain specific embodiments, Y₀ and Y₄ are independently selected from hydrogen, alkyl (e.g., 1C-10C alkyl), cycloalkyl (e.g., 3C-6C cycloalkyl), O-alkyl (e.g., O—(2C-10C)alkyl, —C(O)O-alkyl (e.g., —C(O)O-(2C-10C)alkyl), or phenyl, any of which is optionally substituted with one or more fluorine.

In some embodiments, Y₁ and Y₂ are independently selected from a covalent bond, alkyl, preferably at present a (1C-10C)alkyl, —C(O)O-alkyl, preferably at present —C(O)O-(2C-10C)alkyl, —OC(O)alkyl, preferably at present —OC(O)-(2C-10C)alkyl, O-alkyl, preferably at present —O(2C-10C)alkyl and —S-alkyl, preferably at present —S-(2C-10C)alkyl. In certain embodiments, Y₃ is selected from a covalent bond, alkyl, preferably at present (1C-5C)alkyl and phenyl.

In some embodiments, Z— is present or absent. In certain embodiments, wherein R₁ and/or R₄ is hydrogen, Z— is OH—. In certain embodiments, Z⁻ is any counterion (e.g., one or more counterion), preferably a biocompatible counter ion, such as, by way of non-limiting example, chloride, inorganic or organic phosphate, sulfate, sulfonate, acetate, propionate, butyrate, valerate, caproate, caprylate, caprate, laurate, myristate, palmate, stearate, palmitolate, oleate, linolate, arachidate, gadoleate, vaccinate, lactate, glycolate, salicylate, desamionphenylalanine, desaminoserine, desaminothreonine, ε-hydroxycaproate, 3-hydroxybutylrate, 4-hydroxybutyrate or 3-hydroxyvalerate. In some embodiments, when each Y, R and optional fluorine is covalently bonded to a carbon of the selected backbone, any carbons that are not fully substituted are completed with the appropriate number of hydrogen atoms. The numbers m, n, p, q and r represent the mole fraction of each constitutional unit in its block and v and w provide the molecular weight of each block.

In certain embodiments,
A₀, A₁, A₂, A₃ and A₄ are selected from the group consisting of —C—, —C—C—, —C(O)(CR₁₂R₁₃)ₐC(O)O—, —O(CR₁₂R₁₃)ₐC(O)— and O(CR₁₂R₁₃)_{b}O; wherein,
   a is 1-4;
   b is 2-4;
R₁, R₂, R₃, R₄, R₅, R₆, R₇, R₈, R₉, R₁₀, R₁₁, R₁₂, and R₁₃ are independently selected from the group consisting of hydrogen, (1C-5C)alkyl, (3C-6C)cycloalkyl, (5C-10C)aryl, (4C-10C)heteroaryl, any of which may be optionally substituted with one or more fluorine atoms;
Y₀ and Y₄ are independently selected from the group consisting of hydrogen, (1C-10C)alkyl, (3C-6C)cycloalkyl, O—(1C-10C)alkyl, —C(O)O(1C-10C)alkyl and phenyl, any of which is optionally substituted with one or more fluorine groups;
Y₁ and Y₂ are independently selected from the group consisting of a covalent bond, (1C-10C)alkyl-, —C(O)O(2C-10C) alkyl-, —OC(O)(1C-10C)alkyl-, —O(2C-10C)alkyl- and —S(2C-10C)alkyl-;
Y₃ is selected from the group consisting of a covalent bond, (1C-5C)alkyl and phenyl; wherein tetravalent carbon atoms of A₁-A₄ that are not fully substituted with R₁-R₅ and Y₀-Y₄ are completed with an appropriate number of hydrogen atoms;
Z is one or more physiologically acceptable counterions,
m is 0 to about 0.49;
n is about 0.51 to about 1.0; wherein
   m+n=1
p is about 0.2 to about 0.5;
q is about 0.2 to about 0.5; wherein:
   p is substantially the same as q;
r is 0 to about 0.6; wherein
   p+q+r=1
v is from about 1 to about 25 kDa, or about 5 to about 25 kDa; and,
w is from about 1 to about 50 kDa, or about 5 to about 50 kDa.

In a specific embodiment,
A₀, A₁, A₂, A₃ and A₄ are independently selected from the group consisting of —C—C—, —C(O)(C)ₐC(O)O—, —O(C)ₐC(O)— and —O(C)_{b}O—; wherein,
a is 1-4;
b is 2-4;
R₁, R₂, R₃, R₄, R₅, R₆, R₇, R₈, R₉, R₁₀ and R₁₁ are independently selected from the group consisting of hydrogen, (1C-5C)alkyl, (3C-6C)cycloalkyl and phenyl, any of which may be optionally substituted with one or more fluorine atoms;
Y₀ and Y₄ are independently selected from the group consisting of hydrogen, (1C-10C)alkyl, (3C-6C)cycloalkyl, O—(1C-10C)alkyl, —C(O)O(1C-10C)alkyl and phenyl, any of which is optionally substituted with one or more fluorine groups;
Y₁ and Y₂ are independently selected from the group consisting of a covalent bond, (1C-10C)alkyl-, —C(O)O(2C-10C) alkyl-, —OC(O)(1C-10C) alkyl-, —O(2C-10C)alkyl- and —S(2C-10C)alkyl-;
Y₃ is selected from the group consisting of a covalent bond, (1C-5C)alkyl and phenyl;
wherein tetravalent carbon atoms of A₁-A₄ that are not fully substituted with R₁-R₅ and Y₀-Y₄ are completed with an appropriate number of hydrogen atoms;
Z is a physiologically acceptable counterion,
m is 0 to about 0.49;
n is about 0.51 to about 1.0;
   wherein m+n=1
p is about 0.2 to about 0.5;
q is about 0.2 to about 0.5; wherein:
   p is substantially the same as q;
r is 0 to about 0.6; wherein
   p+q+r=1
v is from about 5 to about 25 kDa; and
w is from about 5 to about 25 kDa.

In some embodiments,
A₁ is —C—C—
Y₁ is —C(O)OCH₂CH₂—;
R₆ is hydrogen;
R₇ and R₈ are each —CH₃; and,
R₂ is —CH₃.

In some embodiments,
A₂ is —C—C—;
Y₂ is —C(O)OCH₂CH₂—;
R₉ is hydrogen;
R₁₀ and R₁₁ are each —CH₃; and,
R₃ is —CH₃.

In some embodiments,
A₃ is —C—C—;
R₄ is CH₃CH₂CH₂—;
Y₃ is a covalent bond;
and Z⁻ is a physiologically acceptable anion.

In some embodiments,
A₄ is —C—C—;
R₅ is selected from the group consisting of hydrogen and —CH₃; and,
Y₄ is —C(O)O(CH₂)₃CH₃.

In some embodiments,
A₀ is C—C—
R₁ is selected from the group consisting of hydrogen and (1C-3C)alkyl; and,
Y₀ is selected from the group consisting of —C(O)O(1C-3C)alkyl.

In some embodiments, m is 0.

In some embodiments, r is 0.

In some embodiments, m and r are both 0.

In certain embodiments, the block copolymer is a diblock copolymer, having the chemical formula (at normal physiological or about neutral pH) of Formula IV1:

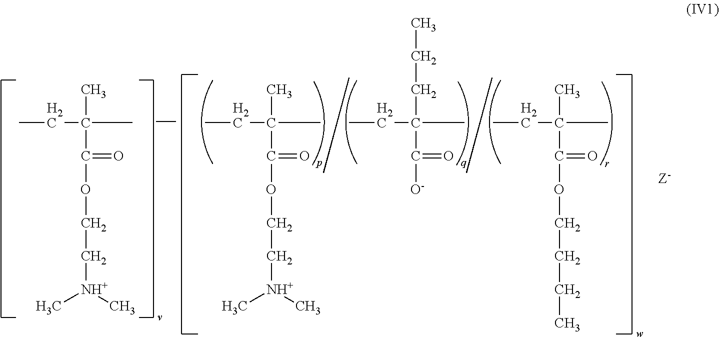

In certain instances, the constitutional units of the compound IV1 are as shown within the square bracket on the left and the curved brackets on the right and they are derived from the monomers:

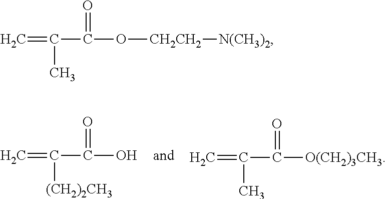

The letters p, q and r represent the mole fraction of each constitutional unit within its block. The letters v and w represent the molecular weight (number average) of each block in the diblock copolymer.

Provided in some embodiments, a compound provided herein is a compound having the structure:

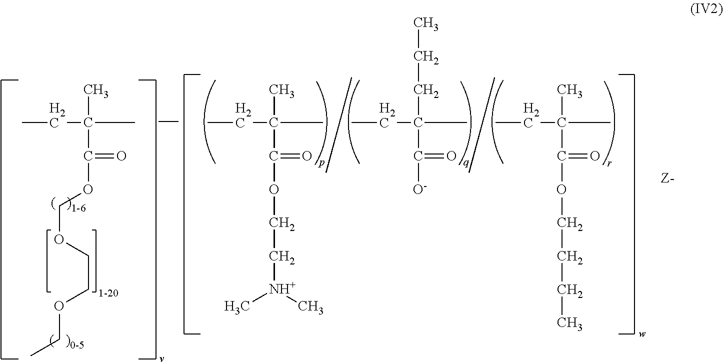

As discussed above, letters p, q and r represent the mole fraction of each constitutional unit within its block. The letters v and w represent the molecular weight (number average) of each block in the diblock copolymer.

In some embodiments, provided herein the following polymers:

[DMAEMA]ᵥ-[Bₚ—/—P_{q}-/-Dᵣ]_{w} IV3

[PEGMA]ᵥ-[Bₚ—/—P_{q}-/-Dᵣ]_{w} IV4

[PEGMAₘ-/-DMAEMAₙ]ᵥ-[Bₚ—/—P_{q}-/-Dᵣ]_{w} IV5

[PEGMAₘ-/-MAA(NHS)ₙ]ᵥ-[Bₚ—/—P_{q}-/-Dᵣ]_{w} IV6

[DMAEMAₘ-/-MAA(NHS)ₙ]ᵥ-[Bₚ—/—P_{q}-/-Dᵣ]_{w} IV7

[HPMAₘ-/-PDSMₙ]ᵥ-[Bₚ—/—P_{q}-/-Dᵣ]_{w} IV8

[PEGMAₘ-/-PDSMₙ]ᵥ-[Bₚ—/—P_{q}-/-Dᵣ]_{w} IV9

In some embodiments, B is butyl methacrylate residue; P is propyl acrylic acid residue; D and DMAEMA are dimethylaminoethyl methacrylate residue; PEGMA is polyethyleneglycol methacrylate residue (e.g., with 1-20 ethylene oxide units, such as illustrated in compound IV2, or 4-5 ethylene oxide units, or 7-8 ethylene oxide units); MAA(NHS) is methylacrylic acid-N-hydroxy succinamide residue; HPMA is N-(2-hydroxypropyl)methacrylamide residue; and PDSM is pyridyl disulfide methacrylate residue. In certain embodiments, the terms m, n, p, q, r, w and v are as described herein. In specific embodiments, w is about 1× to about 5×v.

Compounds of Formulas IV1-IV9 are examples of polymers provided herein comprising a variety of constitutional unit(s) making up the first block of the polymer. In some embodiments, the constitutional unit(s) of the first block are varied or chemically treated in order to create polymers where the first block is or comprises a constitutional unit that is neutral (e.g., PEGMA), cationic (e.g., DMAEMA), anionic (e.g., PEGMA-NHS, where the NHS is hydrolyzed to the acid, or acrylic acid), ampholytic (e.g., DMAEMA-NHS, where the NHS is hydrolyzed to the acid), or zwitterionic (for example, poly[2-methacryloyloxy-2′ trimethylammoniumethyl phosphate]). In some embodiments, polymers comprising pyridyl disulfide functionality in the first block, e.g., [PEGMA-PDSM]-[B—P-D], that can be and is optionally reacted with a thiolated siRNA to form a polymer-siRNA conjugate.

In a specific embodiment, a compound of Formula IV3 is a polymer of the P7 class, as described herein, and has the molecular weight, polydispersity, and monomer composition as set forth in Table 1.

TABLE 1
Molecular weights, polydispersities, and monomer
compositions for a species of P7 polymer

| Polymer Class | P7 |
|---|---|
| Mn of “v” block^{a} | 9100 |
| Mn of “w” block^{a} | 11300 |
| PDI | 1.45 |
| Theoretical % BMA | 40 |
| residue of “w” block | |
| Theoretical % PPA | 30 |
| residue of “w” block | |
| Theoretical % DMAEMA | 30 |
| residue of “w” block | |
| Experimental % BMA | 48 |
| residue of “w” block^{b} | |
| Experimental % PPA | 29 |
| residue of “w” block^{b} | |
| Experimental % DMAEMA | 23 |
| residue of “w” block^{b} | |

^{a}As determined by SEC Tosoh TSK-GEL R-3000 and R-4000 columns (Tosoh Bioscience, Montgomeryville, PA) connected in series to a Viscotek GPCmax VE2001 and refractometer VE3580 (Viscotek, Houston, TX). HPLC-grade DMF containing 0.1 wt % LiBr was used as the mobile phase. The molecular weights of the synthesized copolymers were determined using a series of poly(methyl methacrylate) standards.
^{b}As determined by ¹H NMR spectroscopy (3 wt % in CDCL₃; Bruker DRX 499)

In some specific embodiments, a polymer of Formula IV3 is a polymer of the P7 class according to Table 2.

TABLE 2

| | | Block Ratio | Particle Size |
|---|---|---|---|
| Polymer | Structure | (w/v) | (nm) |
| PRx-1 | [D]_{11.3K}-[B₅₀-P₃₀-D₂₀]_{20.7K} | 1.83 | 41 |
| PRx-2 | [D]_{14.5K}-[B₅₇-P₂₃-D₂₀]_{26.4K} | 1.82 | 49 |
| PRx-3 | [D]_{11.5K}-[B₃₅-P₂₇-D₃₈]_{33.4K} | 2.92 | 60 |
| PRx-4 | [D]_{10.7K}-[B₅₀-P₂₇-D₂₃]_{33.8K} | 3.16 | 50 |
| PRx-5 | [D]_{10.7K}-[B₄₀-P₃₁-D₂₉]_{32.2K} | 3.00 | 59 |
| PRx-6 | [D]_{14.5K}-[B₅₃-P₃₁-D₁₆]_{67.0K} | 4.62 | 115 |

In some specific embodiments, a polymer of Formula IV3 is a polymer of the P7 class called P7v6. PRx0729v6 is used interchangeably with P7v6 in this application and in various priority applications.

### Membrane Destabilizing Block Copolymers

In one embodiment, micelles provided herein, or the component parts thereof, are membrane-destabilizing (e.g., comprise a membrane destabilizing block, group, moiety, or the like). In further or alternative embodiments, the plurality of block copolymers form a shell and a core of a micelle. In specific embodiments, the micelle comprises a hydrophilic and/or charged shell. In further or alternative embodiments, the micelle comprises a substantially hydrophobic core (e.g., the core comprises hydrophobic groups, monomeric units, moieties, blocks, or the like). In specific embodiments, one or more of the block copolymers each comprise (1) a hydrophilic, charged block forming the shell of the micelle; and (2) a substantially hydrophobic block forming the core of the micelle. In some embodiments, one or more of the block copolymers comprise a plurality of first chargeable species and a plurality of hydrophobicity enhancers. In specific embodiments, the first chargeable species are anionic chargeable species (e.g., are or become charged at a specific pH). In further embodiments, the one or more of the block copolymers comprise a second chargeable species. (i.e., the hydrophilic block may have more than one different type of anionic species) In certain embodiments, the micelle comprises at least one polynucleotide (e.g., oligonucleotide). In specific embodiments, the polynucleotide (e.g., oligonucleotide) is not in the core of the micelle.

In some embodiments, a membrane-destabilizing block copolymer comprises (i) a plurality of hydrophobic monomeric residues, (ii) a plurality of anionic monomeric residues having a chargeable species, the chargeable species being anionic at physiological pH, and being substantially neutral or non-charged at an endosomal pH and (iii) optionally a plurality of cationic monomeric residues. In some embodiments, the combination of two mechanisms of membrane disruption, (a) a polycation (such as DMAEMA) and (b) a hydrophobized polyanion (such as propylacrylic acid), acting together have an additive or synergistic effect on the potency of the membrane destabilization conferred by the polymer.

In some embodiments, modification of the ratio of anionic to cationic species in a block copolymer allows for modification of membrane destabilizing activity of a micelle described herein. In some of such embodiments, the ratio of anionic:cationic species in a block copolymer ranges from about 4:1 to about 1:4 at physiological pH. In some of such embodiments, modification of the ratio of anionic to cationic species in a hydrophobic block of a block copolymer allows for modification of membrane destabilizing activity of a micelle described herein. In some of such embodiments, the ratio of anionic:cationic species in a hydrophobic block of a block copolymer described herein ranges from about 1:2 to about 3:1, or from about 1:1 to about 2:1 at serum physiological pH.

In certain embodiments, the membrane destabilizing block copolymers present in a micelle provided herein comprise a core section (e.g., core block) that comprises a plurality of hydrophobic groups. In more specific embodiments, the core section (e.g., core block) comprises a plurality of hydrophobic groups and a plurality of first chargeable species or groups. In still more specific embodiments, such first chargeable species or groups are negatively charged and/or are chargeable to a negatively charged species or group (e.g., at about a neutral pH, or a pH of about 7.4). In some specific embodiments, the core section (e.g., core block) comprises a plurality of hydrophobic groups, a plurality of first chargeable species or groups, and a plurality of second chargeable species or groups. In more specific embodiments, the first chargeable species or groups are negatively charged and/or are chargeable to a negatively charged species or group, and the second chargeable species or groups are positively charged and/or are chargeable to a positively charged species or group (e.g., at about a neutral pH, or a pH of about 7.4).

### Ratio of Hydrophilic Block to Hydrophobic Block

In certain embodiments, micelles provided herein are further or alternatively characterized by other criteria: (1) the molecular weight of the individual blocks and their relative length ratios is decreased or increased in order to govern the size of the micelle formed and its relative stability and (2) the size of the polymer hydrophilic block is varied (e.g., by varying the number of cationic monomers) in order to provide effective complex formation with and/or charge neutralization of an anionic therapeutic agent (e.g., an oligonucleotide drug).

In some embodiments, the block ratio of a number-average molecular weight (Mn) of the hydrophilic block to the hydrophobic block is from about 1:1 to about 1:10. In some embodiments, micelles described herein comprise copolymers with a block ratio of a number-average molecular weight (Mn) of the hydrophilic block to the hydrophobic block from about 1:1 to about 1:5, or from about 1:1 to about 1:2.5.

In some embodiments, the block ratio of a number-average molecular weight (Mn) of the hydrophilic block to the hydrophobic block is from about 1:1 to about 10:1. In some embodiments, micelles described herein comprise copolymers with a block ratio of a number-average molecular weight (Mn) of the hydrophilic block to the hydrophobic block from about 1:1 to about 5:1, or from about 1:1 to about 2.5:1.

### Polymer Architecture

In specific instances, provided herein are the block copolymers of the following structure:

α-[Dₛ-Xₜ]_{b}—[Bₓ—P_{y}-D_{z}]ₐ-ω [Structure 1]

α-[Bₓ—P_{y}-D_{z}]ₐ[Dₛ-Xₜ]_{b}-ω [Structure 2]

wherein x, y, z, s and t are the mole % composition (generally, 0-50%) of the individual monomeric units D (DMAEMA), B (BMA), P (PAA), and a hydrophilic neutral monomer (X) in the polymer block, a and b are the molecular weights of the blocks, [Dₛ-Xₜ] is the hydrophilic block, and α and ω denote the opposite ends of the polymer. In certain embodiments, x is 50%, y is 25% and z is 25%. In certain embodiments, x is 60%, y is 20% and z is 20%. In certain embodiments, x is 70%, y is 15% and z is 15%. In certain embodiments, x is 50%, y is 25% and z is 25%. In certain embodiments, x is 33%, y is 33% and z is 33%. In certain embodiments, x is 50%, y is 20% and z is 30%. In certain embodiments, x is 20%, y is 40% and z is 40%. In certain embodiments, x is 30%, y is 40% and z is 30%.

In some embodiments, a block copolymer described herein comprises a hydrophilic block of about 2,000 KDa to about 30,000 KDa, about 5,000 KDa to about 20,000 KDa, or about 7,000 KDa to about 15,000 KDa. In specific embodiments, the hydrophilic block is of about 7,000 KDa, 8,000 KDa, 9,000 KDa, 10,000 KDa, 11,000 KDa, 12,000 KDa, 13,000 KDa, 14,000 KDa, or 15,000 KDa. In certain embodiments, a block copolymer described herein comprises a hydrophobic block of about 10,000 KDa to about 100,000 KDa, about 15,000 KDa to about 35,000 KDa, or about 20,000 KDa to about 30,000 KDa. In some specific embodiments, a block copolymer comprising a hydrophilic block of 12,500 KDa and a hydrophobic block of 25,000 KDa (length ratio of 1:2) forms a micelle. In some specific embodiments, a block copolymer comprising a hydrophilic block of 10,000 KDa and a hydrophobic block of 30,000 KDa (length ratio of 1:3) forms a micelle.

In some specific embodiments, a block copolymer comprising a hydrophilic block of 10,000 KDa and a hydrophobic block of 25,000 Kda (length ratio of 1:2.5) forms a micelle of approximately 45 nm (as determined by dynamic light scattering measurements or electron microscopy). In some specific embodiments, the micelles are 80 or 130 nm (as determined by dynamic light scattering measurements or electron microscopy). Typically, as the molecular weight (or length) of [Dₛ-Xₜ], which forms the micelle shell, increases relative to —[Bₓ—P_{y}-D_{z}], the hydrophobic block that forms the core, the size of the micelle increases. In some instances, the size of the polymer cationic block that forms the shell ([Dₛ-Xₜ] is important in providing effective complex formation/charge neutralization with the oligonucleotide drug. For example, in certain instances, for siRNA of approximately 20 base pairs (i.e., 40 anionic charges) a cationic block has a length suitable to provide effective binding, for example 40 cationic charges. For a shell block containing 80 DMAEMA monomers (MW=11,680) with a pKa value of 7.4, the block contains 40 cationic charges at pH 7.4. In some instances, stable polymer-siRNA conjugates (e.g., complexes) form by electrostatic interactions between similar numbered opposite charges. In certain instances, avoiding a large number of excess positive charge helps to prevent significant in vitro and in vivo toxicity.

### Polydispersity

In some embodiments, block copolymers utilized in the micelles provided herein have a low polydispersity index (PDI) or differences in chain length. Polydispersity index (PDI) is determined in any suitable manner, e.g., by dividing the weight average molecular weight of the polymer chains by their number average molecular weight. The number average molecule weight is the sum of individual chain molecular weights divided by the number of chains. The weight average molecular weight is proportional to the square of the molecular weight divided by the number of molecules of that molecular weight. Since the weight average molecular weight is always greater than the number average molecular weight, polydispersity is always greater than or equal to one. As the numbers come closer and closer to being the same, i.e., as the polydispersity approaches a value of one, the polymer becomes closer to being monodisperse in which every chain has exactly the same number of monomeric units. Polydispersity values approaching one are achievable using living radical polymerization. Methods of determining polydispersity, such as, but not limited to, size exclusion chromatography, dynamic light scattering, matrix-assisted laser desorption/ionization chromatography and electrospray mass chromatography are well known in the art. In some embodiments, block copolymer of the micellar assemblies provided herein have a polydispersity index (PDI) of less than 2.0, or less than 1.5, or less than 1.4, or less than 1.3, or less than 1.2.

### Synthesis

In certain embodiments, block copolymers comprise ethylenically unsaturated monomers. The term “ethylenically unsaturated monomer” is defined herein as a compound having at least one carbon double or triple bond. The non-limiting examples of the ethylenically unsaturated monomers are: an alkyl(alkyl)acrylate, a methacrylate, an acrylate, an alkylacrylamide, a methacrylamide, an acrylamide, a styrene, an allylamine, an allylammonium, a diallylamine, a diallylammonium, an N-vinyl formamide, a vinyl ether, a vinyl sulfonate, an acrylic acid, a sulfobetaine, a carboxybetaine, a phosphobetaine, or maleic anhydride.

In some embodiments, monomers suitable for use in the preparation of the block copolymers provided herein include, by way of non-limiting example, one or more of the following monomers: methyl methacrylate, ethyl acrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate (DMAEMA), triethyleneglycol methacrylate, oligoethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, oligoethyleneglycol acrylate, methacrylamide, N-methylacrylamide, N,N-dimethylacrylamide, N-tert-butylmethacrylamide, N-n-butylmethacrylamide, N-methylolacrylamide, N-ethylolacrylamide, vinyl benzoic acid (all isomers), diethylaminostyrene (all isomers), alpha-methylvinyl benzoic acid (all isomers), diethylamino alpha-methylstyrene (all isomers), p-vinylbenzenesulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropylmethacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, vinyl acetate, vinyl butyrate, vinyl benzoate, vinyl chloride, vinyl fluoride, vinyl bromide, maleic anhydride, N-arylmaleimide, N-phenylmaleimide, N-alkylmaleimide, N-butylmaleimide, N-vinylpyrrolidone, N-vinylcarbazole, butadiene, isoprene, chloroprene, ethylene, propylene, 1,5-hexadienes, 1,4-hexadienes, 1,3-butadienes, 1,4-pentadienes, vinylalcohol, vinylamine, N-alkylvinylamine, allylamine, N-alkylallylamine, diallylamine, N-alkyldiallylamine, alkylenimine, acrylic acids, alkylacrylates, acrylamides, methacrylic acids, alkylmethacrylates, methacrylamides, N-alkylacrylamides, N-alkylmethacrylamides, styrene, N-isopropylacrylamide, vinylnaphthalene, vinyl pyridine, ethylvinylbenzene, aminostyrene, vinylpyridine, vinylimidazole, vinylbiphenyl, vinylanisole, vinylimidazolyl, vinylpyridinyl, vinylpolyethyleneglycol, dimethylaminomethylstyrene, trimethylammonium ethyl methacrylate, trimethylammonium ethyl acrylate, dimethylamino propylacrylamide, trimethylammonium ethylacrylate, trimethylammonium ethyl methacrylate, trimethylammonium propyl acrylamide, dodecyl acrylate, octadecyl acrylate, or octadecyl methacrylate monomers, or combinations thereof.

In some embodiments, functionalized versions of these monomers are optionally used. A functionalized monomer, as used herein, is a monomer comprising a masked or non-masked functional group, e.g. a group to which other moieties can be attached following the polymerization. The non-limiting examples of such groups are primary amino groups, carboxyls, thiols, hydroxyls, azides, and cyano groups. Several suitable masking groups are available (see, e.g., T. W. Greene & P. G. M. Wuts, Protective Groups in Organic Synthesis (2nd edition) J. Wiley & Sons, 1991 and P. J. Kocienski, Protecting Groups, Georg Thieme Verlag, 1994, which are incorporated by reference for such disclosure).

Polymers described here are prepared in any suitable manner. Suitable synthetic methods used to produce the polymers provided herein include, by way of non-limiting example, cationic, anionic and free radical polymerization. In some instances, when a cationic process is used, the monomer is treated with a catalyst to initiate the polymerization. Optionally, one or more monomers are used to form a copolymer. In some embodiments, such a catalyst is an initiator, including, e.g., protonic acids (Bronsted acid) or Lewis acids, in the case of using Lewis acid some promoter such as water or alcohols are also optionally used. In some embodiments, the catalyst is, by way of non-limiting example, hydrogen iodide, perchloric acid, sulfuric acid, phosphoric acid, hydrogen fluoride, chlorosulfonic acid, methansulfonic acid, trifluoromethanesulfonic acid, aluminum trichloride, alkyl aluminum chlorides, boron trifluoride complexes, tin tetrachloride, antimony pentachloride, zinc chloride, titanium tetrachloride, phosphorous pentachloride, phosphorus oxychloride, or chromium oxychloride. In certain embodiments, polymer synthesis is performed neat or in any suitable solvent. Suitable solvents include, but are not limited to, pentane, hexane, dichloromethane, chloroform, or dimethyl formamide (DMF). In certain embodiments, the polymer synthesis is performed at any suitable reaction temperature, including, e.g., from about −50° C. to about 100° C., or from about 0° C. to about 70° C.

In certain embodiments, the block copolymers are prepared by the means of a free radical polymerization. When a free radical polymerization process is used, (i) the monomer, (ii) optionally, the co-monomer, and (iii) an optional source of free radicals are provided to trigger a free radical polymerization process. In some embodiments, the source of free radicals is optional because some monomers may self-initiate upon heating at high temperature. In certain instances, after forming the polymerization mixture, the mixture is subjected to polymerization conditions. Polymerization conditions are those conditions that cause at least one monomer to form at least one polymer, as discussed herein. Such conditions are optionally varied to any suitable level and include, by way of non-limiting example, temperature, pressure, atmosphere, ratios of starting components used in the polymerization mixture and reaction time. The polymerization is carried out in any suitable manner, including, e.g., in solution, dispersion, suspension, emulsion or bulk.

In some embodiments, initiators are present in the reaction mixture. Any suitable initiator is optionally utilized if useful in the polymerization processes described herein. Such initiators include, by way of non-limiting example, one or more of alkyl peroxides, substituted alkyl peroxides, aryl peroxides, substituted aryl peroxides, acyl peroxides, alkyl hydroperoxides, substituted alkyl hydroperoxides, aryl hydroperoxides, substituted aryl hydroperoxides, heteroalkyl peroxides, substituted heteroalkyl peroxides, heteroalkyl hydroperoxides, substituted heteroalkyl hydroperoxides, heteroaryl peroxides, substituted heteroaryl peroxides, heteroaryl hydroperoxides, substituted heteroaryl hydroperoxides, alkyl peresters, substituted alkyl peresters, aryl peresters, substituted aryl peresters, or azo compounds. In specific embodiments, benzoylperoxide (BPO) and/or AIBN are used as initiators.

In some embodiments, polymerization processes are carried out in a living mode, in any suitable manner, such as but not limited to Atom Transfer Radical Polymerization (ATRP), nitroxide-mediated living free radical polymerization (NMP), ring-opening polymerization (ROP), degenerative transfer (DT), or Reversible Addition Fragmentation Transfer (RAFT). Using conventional and/or living/controlled polymerizations methods, various polymer architectures can be produced, such as but not limited to block, graft, star and gradient copolymers, whereby the monomer units are either distributed statistically or in a gradient fashion across the chain or homopolymerized in block sequence or pendant grafts. In other embodiments, polymers are synthesized by Macromolecular design via reversible addition-fragmentation chain transfer of Xanthates (MADIX) (Direct Synthesis of Double Hydrophilic Statistical Di- and Triblock Copolymers Comprised of Acrylamide and Acrylic Acid Units via the MADIX Process”, Daniel Taton, et al., Macromolecular Rapid Communications, 22, No. 18, 1497-1503 (2001).)

In certain embodiments, Reversible Addition-Fragmentation chain Transfer or RAFT is used in synthesizing ethylenic backbone polymers of this invention. RAFT is a living polymerization process. RAFT comprises a free radical degenerative chain transfer process. In some embodiments, RAFT procedures for preparing a polymer described herein employs thiocarbonylthio compounds such as, without limitation, dithioesters, dithiocarbamates, trithiocarbonates and xanthates to mediate polymerization by a reversible chain transfer mechanism. In certain instances, reaction of a polymeric radical with the C═S group of any of the preceding compounds leads to the formation of stabilized radical intermediates. Typically, these stabilized radical intermediates do not undergo the termination reactions typical of standard radical polymerization but, rather, reintroduce a radical capable of re-initiation or propagation with monomer, reforming the C═S bond in the process. In most instances, this cycle of addition to the C═S bond followed by fragmentation of the ensuing radical continues until all monomer has been consumed or the reaction is quenched. Generally, the low concentration of active radicals at any particular time limits normal termination reactions.

Polymerization processes described herein optionally occur in any suitable solvent or mixture thereof. Suitable solvents include water, alcohol (e.g., methanol, ethanol, n-propanol, isopropanol, butanol), tetrahydrofuran (THF) dimethyl sulfoxide (DMSO), dimethylformamide (DMF), acetone, acetonitrile, hexamethylphosphoramide, acetic acid, formic acid, hexane, cyclohexane, benzene, toluene, dioxane, methylene chloride, ether (e.g., diethyl ether), chloroform, and ethyl acetate. In one aspect, the solvent includes water, and mixtures of water and water-miscible organic solvents such as DMF.

In some embodiments, a conjugatable group is introduced at the a end of the polymer provided herein by preparing the polymer in the presence of a chain transfer reagent comprising a conjugatable group (e.g., an azide or a pyridyl disulfide group) wherein the conjugatable group is compatible with the conditions of the polymerization process. A non-limiting example of such chain transfer reagent is described by Heredia, K. L et al (see Chem. Commun., 2008, 28, 3245-3247, which is incorporated by reference for the disclosure). In some embodiments, the chain transfer reagent comprises a masked conjugatable group which, following an unmasking reaction, is linked to a siRNA agent or a targeting agent. In some embodiments, a targeting agent, such as but not limited to a small molecule targeting agent (e.g., biotin residue or monosaccharide), is attached at the a end of the polymer provided herein by preparing the polymer in the presence of chain transfer reagent wherein the chain transfer reagent comprises the targeting agent.

In some instances, the block copolymers comprise conjugatable monomers (e.g., monomers bearing conjugatable groups) which is used for post-polymerization introduction of additional functionalities (e.g. small molecule targeting agents) via know in the art chemistries, for example, “click” chemistry (for example of “click” reactions, see Wu, P.; Fokin, V. V. Catalytic Azide-Alkyne Cycloaddition: Reactivity and Applications. Aldrichim. Acta, 2007, 40, 7-17, which is incorporated by reference). In some embodiments, a monomer comprising such conjugatable groups is co-polymerized with a hydrophobic monomer and a monomer comprising a chargeable to anion species. In some instances, N-hydroxysuccinimide ester of acrylic or alkylacrylic acid is copolymerized with other monomers to form a copolymer which is reacted with amino-functionalized molecules, e.g. targeting ligands or amino derivatives of PEGs. In some embodiments, the monomer comprising a conjugatable group is a pyridyldisulfide acrylate (PDSA).

In certain embodiments, the block copolymer comprises a PEG substituted monomeric unit (e.g., the PEG is a side chain and does not comprise the backbone of the polynucleotide carrier block). In some instances, one or more of the polymers described herein comprise polyethyleneglycol (PEG) chains or blocks with molecular weights of approximately from 1,000 to approximately 30,000. In some embodiments, PEG is conjugated to polymer ends groups, or to one or more pendant modifiable group present in a polymer of a polymeric carrier provided herein. In some embodiments, PEG residues are conjugated to modifiable groups within the hydrophilic segment or block (e.g., a shell block) of a polymer (e.g., block copolymer) of a polymeric carrier provided herein. In certain embodiments, a monomer comprising a PEG residue of 2-20 ethylene oxide units is co-polymerized to form the hydrophilic portion of the polymer forming the polymeric carrier provided herein.

### Micellar Payload: Polynucleotides

Provided herein are micelles that deliver diagnostic and/or therapeutic agents (including, e.g., oligonucleotides) to a living cell. In some embodiments, the micelles comprise a plurality of block copolymers and optionally at least one therapeutic agent (e.g., a polynucleotide, e.g., siRNA). The micelles provided herein are biocompatible, stable (including chemically and/or physically stable), and/or reproducibly synthesized. Preferably, the micelles provided herein are non-toxic (e.g., exhibit low toxicity), protect the therapeutic agent (e.g., oligonucleotide) payload from degradation, enter living cells via a naturally occurring process (e.g., by endocytosis), and/or deliver the therapeutic agent (e.g., oligonucleotide) payload into the cytoplasm of a living cell after being contacted with the cell.

In certain instances, the polynucleotide (e.g., oligonucleotide) is an siRNA and/or another ‘nucleotide-based’ agent that alters the expression of at least one gene in the cell. Accordingly, in certain embodiments, the micelles provided herein are useful for delivering siRNA into a cell. In certain instances, the cell is in vitro, and in other instances, the cell is in vivo (e.g., a mouse or a human). In some embodiments, a therapeutically effective amount of the micelles comprising an siRNA is administered to an individual in need thereof (e.g., in need of having a gene knocked down, wherein the gene is capable of being knocked down by the siRNA administered). In specific instances, the micelles are useful for or are specifically designed for delivery of siRNA to specifically targeted cells of the individual.

In some embodiments, the micelles provided herein deliver RNAi agents (e.g., siRNA) to an individual in need thereof. In certain of such embodiments provided herein is a micelle comprising a polymer bioconjugate, e.g., an RNAi agent conjugated (e.g., ionically or covalently) to a block copolymer. In more specific embodiments, the RNAi agent is conjugated to the alpha end of the block copolymer, and in other specific embodiments, the RNAi agent is conjugated to the omega end of the block copolymer. In some embodiments, siRNA is covalently conjugated to the pendant side chains of one or more polymer's monomeric units.

In some embodiments, the RNAi molecule is a polynucleotide. In certain embodiments, the polynucleotide is an oligonucleotide gene expression modulator. In further embodiments, the polynucleotide is an oligonucleotide knockdown agent or the RNAi agent. In specific embodiments, the polynucleotide is a dicer substrate or siRNA.

In certain embodiments, the polynucleotide comprises 5′ and a 3′ end and is coupled to the membrane-destabilizing polymer at either the 5′ or 3′ end of the polynucleotide. In various embodiments, RNAi agent is covalently coupled to the block co polymer through a linking moiety.

In some embodiments, the linking moiety comprises an affinity binder pair. In certain embodiments, a polynucleotide and/or one of the ends of the pH-dependent membrane destabilizing polymer is modified with chemical moieties that afford a polynucleotide and/or a polymer that have an affinity for one another, such as arylboronic acid-salicylhydroxamic acid, leucine zipper or other peptide motifs, or other types of chemical affinity linkages.

The linking moiety (e.g., a covalent bond) between a block copolymer and an RNAi agent of a micelle described herein is, optionally, non-cleavable, or cleavable. In certain embodiments, a precursor of an RNAi agent (e.g. a dicer substrate) is attached to the polymer (e.g., the alpha or omega end conjugatable group of the polymer) by a non-cleavable linking moiety. In some embodiments, an RNAi agent is attached through a cleavable linking moiety. In some instances, the linking moiety between the RNAi agent and the polymer of the micelle provided herein comprises a cleavable bond. In other instances, the linking moiety between the RNAi agent and the polymer of the micelle provided herein is non-cleavable. In certain embodiments, the cleavable bonds utilized in the micelles described herein include, by way of non-limiting example, disulfide bonds (e.g., disulfide bonds that dissociate in the reducing environment of the cytoplasm). In some embodiments, the linking moiety is cleavable and/or comprises a bond that is cleavable in endosomal conditions. In some embodiments, the linking moiety is cleavable and/or comprises a bond that is cleavable by a specific enzyme (e.g., a phosphatase, or a protease). In some embodiments, the linking moiety is cleavable and/or comprises a bond that is cleavable upon a change in an intracellular parameter (e.g., pH, redox potential). In some embodiments, covalent association between a polymer (e.g., the alpha or omega end conjugatable group of the polymer) and an RNAi agent (e.g., an oligonucleotide or siRNA) is achieved through any suitable chemical conjugation method, including but not limited to amine-carboxyl linkers, amine-aldehyde linkers, amine-ketone linkers, amine-carbohydrate linkers, amine-hydroxyl linkers, amine-amine linkers, carboxyl-sulfhydryl linkers, carboxyl-carbohydrate linkers, carboxyl-hydroxyl linkers, carboxyl-carboxyl linkers, sulfhydryl-carbohydrate linkers, sulfhydryl-hydroxyl linkers, sulfhydryl-sulfhydryl linkers, carbohydrate-hydroxyl linkers, carbohydrate-carbohydrate linkers, and hydroxyl-hydroxyl linkers. In some embodiments, a bifunctional cross-linking reagent is employed to achieve the covalent conjugation between suitable conjugatable groups of RNAi agent and a block co polymer. In some embodiments, conjugation is also performed with pH-sensitive bonds and linkers, including, but not limited to, hydrazone and acetal linkages. In certain embodiments, an RNAi (e.g., a ribooligonucleotide) molecule is covalently linked to a boronic acid functionality (e.g., a phenylboronic acid residue) incorporated into the alpha or the omega end of the polymer through the formation of an ester of the boronic acid with the 2′ and 3′-hydroxyl of the terminal ribose residue of the RNAi agent. Any other suitable conjugation method is optionally utilized as well, for example a large variety of conjugation chemistries are available (see, for example, Bioconjugation, Aslam and Dent, Eds, Macmillan, 1998 and chapters therein).

In certain embodiments, a polymer bioconjugate of a polynucleotide (e.g., siRNA, oligonucleotide) with a block copolymer described herein (e.g., the alpha or omega end conjugatable group of the polymer) is prepared according to a process comprising the following two steps: (1) activating a modifiable end group (for example, 5′- or 3′-hydroxyl or amino group) of an oligonucleotide using any suitable activation reagents, such as but not limited to 1-ethyl-3,3-dimethylaminopropyl carbodiimide (EDAC), imidazole, N-hydrosuccinimide (NHS) and dicyclohexylcarbodiimide (DCC), HOBt (1-hydroxybenzotriazole), p-nitrophenylchloroformate, carbonyldiimidazole (CDI), and N,N′-disuccinimidyl carbonate (DSC); and (2) covalently linking the polymer (e.g., the alpha or omega end of the polymer) to the end of the oligonucleotide. In some embodiments, the 5′- or 3′-end modifiable group of an oligonucleotide is substituted by other functional groups prior to conjugation with the polymer. For example, hydroxyl group (—OH) is optionally substituted with a linker carrying sulfhydryl group (—SH), carboxyl group (—COOH), or amine group (—NH₂).

In yet another embodiment, an oligonucleotide comprising a functional group introduced into one or more of the bases (for example, a 5-aminoalkylpyrimidine), is conjugated to a copolymer comprising a micelle provided herein using a an activating agent or a reactive bifunctional linker according to any suitable procedure. A variety of such activating agents and bifunctional linkers is available commercially from such suppliers as Sigma, Pierce, Invitrogen and others.

In some specific embodiments, a block copolymer is prepared by RAFT polymerization employing a chain-transfer agent comprising a masked conjugatable group. In a specific instance, pyridyl-disulfide comprising CTA is used to synthesize such polymer. The covalent end-conjugation of an RNAi agent is achieved by treating a thiol-comprising RNAi agent with the polymer. In some instances, an excess of a thiol-comprising RNAi agent compared to polymer concentration is used to achieve the conjugation.

In certain embodiments, micelles described herein facilitate intracellular delivery of a bioactive agent (e.g., an antibody, siRNA or the like). In certain embodiments, micelles described herein facilitate intracellular delivery of siRNA that is connected by direct polymer-RNA conjugation. In certain embodiments, a micelle that enhances intracellular delivery of siRNA comprises a first block that enhances water solubility (e.g., a first block that comprises hydrophilic monomers) and/or pharmacokinetic properties, and a second block that is pH-responsive.

### Targeting Moieties

In certain instances, the efficiency of the cell uptake of the micelles is enhanced by incorporation of targeting moieties into the micelle. A “targeting ligand” (used interchangeably with “targeting moiety”) binds to the surface of a cell (e.g., a select cell). In some embodiments, targeting moieties recognize a specific cell surface antigen or bind to a receptor on the surface of the target cell. Suitable targeting ligands include, by way of non-limiting example, antibodies, antibody-like molecules, or peptides, such as an integrin-binding peptides such as RGD-containing peptides, or small molecules, such as vitamins, e.g., folate, sugars such as lactose and galactose, or other small molecules. Cell surface antigens include a cell surface molecule such as a protein, sugar, lipid or other antigen on the cell surface. In specific embodiments, the cell surface antigen undergoes internalization. Examples of cell surface antigens targeted by the targeting moieties of the micelles provided herein include, but are not limited, to the transferrin receptor type 1 and 2, the EGF receptor, HER2/Neu, VEGF receptors, integrins, NGF, CD2, CD3, CD4, CD8, CD19, CD20, CD22, CD33, CD43, CD38, CD56, CD69, and the asialoglycoprotein receptor. A targeting ligand can also comprise an artificial affinity molecule, e.g., a peptidomimetic or an aptamer.

Targeting ligands are attached, in various embodiments, to either end of a polymer (e.g., block copolymer) of the micelle, or to a side chain or a pendant group of a monomeric unit, or incorporated into a polymer. In certain embodiments, a monomer comprising a targeting agent residue (e.g., a polymerizable vinyl monomer comprising a targeting agent) is co-polymerized to form the block copolymer forming a micelle provided herein. In certain embodiments, one or more targeting ligands is coupled to the block copolymer of a micelle provided herein through a linking moiety. In some embodiments, the linking moiety coupling the targeting ligand to the block co polymer is a cleavable linking moiety (e.g., comprises a cleavable bond). In some embodiments, the linking moiety is cleavable and/or comprises a bond that is cleavable in endosomal conditions. In some embodiments, the linking moiety is cleavable and/or comprises a bond that is cleavable by a specific enzyme (e.g., a phosphatase, or a protease). In some embodiments, the linking moiety is cleavable and/or comprises a bond that is cleavable upon a change in an intracellular parameter (e.g., pH, redox potential).

In some embodiments, the targeting agent is a proteinaceous targeting agent (e.g., a peptide, and antibody, an antibody fragment). Attachment of the targeting moiety to the polymer is achieved in any suitable manner, e.g., by any one of a number of conjugation chemistry approaches including but not limited to amine-carboxyl linkers, amine-sulfhydryl linkers, amine-carbohydrate linkers, amine-hydroxyl linkers, amine-amine linkers, carboxyl-sulfhydryl linkers, carboxyl-carbohydrate linkers, carboxyl-hydroxyl linkers, carboxyl-carboxyl linkers, sulfhydryl-carbohydrate linkers, sulfhydryl-hydroxyl linkers, sulfhydryl-sulfhydryl linkers, carbohydrate-hydroxyl linkers, carbohydrate-carbohydrate linkers, and hydroxyl-hydroxyl linkers. In specific embodiments, “click” chemistry is used to attach the targeting ligand to the block copolymers of the micelles provided herein (for example of “click” reactions, see Wu, P.; Fokin, V. V. Catalytic Azide-Alkyne Cycloaddition: Reactivity and Applications. Aldrichim. Acta 2007, 40, 7-17). A large variety of conjugation chemistries are optionally utilized (see, for example, Bioconjugation, Aslam and Dent, Eds, Macmillan, 1998 and chapters therein). In some embodiments, targeting ligands are attached to a monomer and the resulting compound is then used in the polymerization synthesis of a polymer (e.g., copolymer) utilized in a micelle described herein. In some embodiments, the targeting ligand is attached to the sense or antisense strand of siRNA bound to a polymer of the micelle. In certain embodiments, the targeting agent is attached to a 5′ or a 3′ end of the sense or the antisense strand.

In specific embodiments, the micelles provided herein are biocompatible. As used herein, “biocompatible” refers to a property of a compound (e.g., micelle associated with a polynucleotide) characterized by it, or its in vivo degradation products, being not, or at least minimally and/or reparably, injurious to living tissue; and/or not, or at least minimally and controllably, causing an immunological reaction in living tissue. With regard to salts, it is presently preferred that any counterions, (e.g., cationic species or anionic species) be biocompatible. As used herein, “physiologically acceptable” is interchangeable with biocompatible. In some instances, the micelles and/or polymers used therein (e.g., copolymers) exhibit low toxicity compared to cationic lipids.

### Cell Uptake

In some embodiments, the micelles comprising RNAi agents (e.g., oligonucleotides or siRNA) are delivered to cells by endocytosis. Intracellular vesicles and endosomes are used interchangeably throughout this specification. Successful delivery of RNAi agents (e.g., oligonucleotide or siRNA) into the cytoplasm generally has a mechanism for endosomal escape. In certain instances, the micelles comprising RNAi agents (e.g., oligonucleotide or siRNA) provided herein are sensitive to the lower pH in the endosomal compartment upon endocytosis. In certain instances, endocytosis triggers protonation or charge neutralization of chargeable monomeric units or species chargeable to anionic units (e.g., propyl acrylic acid units) or species of the polymers and/or micelles provided herein, resulting in a conformational transition in the polymer. In certain instances, this conformational transition results in a more hydrophobic membrane destabilizing form which mediates release of the therapeutic agent (e.g., oligonucleotide or siRNA) from the endosomes to the cytoplasm. In those micelles comprising siRNA, delivery of siRNA into the cytoplasm allows its mRNA knockdown effect to occur. In those polymer conjugates comprising other types of RNAi agents, delivery into the cytoplasm allows their desired action to occur.

Moreover, in certain embodiments, micelles provided herein selectively uptake small hydrophobic molecules, such as hydrophobic small molecule compounds (e.g., hydrophobic small molecule drugs) into the hydrophobic core of the micelles. In specific embodiments, micelles provided herein selectively uptake small hydrophobic molecules, such as the hydrophobic small molecule compound pyrene into the hydrophobic core of a micelle.

### EXAMPLES

Throughout the description of the present invention, various known acronyms and abbreviations are used to describe monomers or monomeric residues derived from polymerization of such monomers. Without limitation, unless otherwise noted: “BMA” (or the letter “B” as equivalent shorthand notation) represents butyl methacrylate or monomeric residue derived therefrom; “DMAEMA” (or the letter “D” as equivalent shorthand notation) represents N,N-dimethylaminoethyl methacrylate or monomeric residue derived therefrom; “Gal” refers to galactose or a galactose residue, optionally including hydroxyl-protecting moieties (e.g., acetyl) or to a pegylated derivative thereof (as described below); HPMA represents 2-hydroxypropyl methacrylate or monomeric residue derived therefrom; “MAA” represents methylacrylic acid or monomeric residue derived therefrom; “MAA(NHS)” represents N-hydroxyl-succinimide ester of methacrylic acid or monomeric residue derived therefrom; “PAA” (or the letter “P” as equivalent shorthand notation) represents 2-propylacrylic acid or monomeric residue derived therefrom, “PEGMA” refers to the pegylated methacrylic monomer, CH₃—O—(CH₂O)₇₋₈OC(O)C(CH₃)CH₂ or monomeric residue derived therefrom. In each case, any such designation indicates the monomer (including all salts, or ionic analogs thereof), or a monomeric residue derived from polymerization of the monomer (including all salts or ionic analogs thereof), and the specific indicated form is evident by context to a person of skill in the art.

### Example 1

### Preparation of Di-Block Polymers and Copolymers

Di-block polymers and copolymers of the following general formula are prepared:

[A1ₓ-/-A2_{y}]ₙ-[B1ₓ-/-B2_{y}-/-B3_{z}]₁₋₅ₙ

Where [A1-A2] is the first block copolymer, composed of residues of monomers A1 and A2

[B1-B2-B3] is the second block copolymer, composed of residues of monomers B1, B2, B3
x, y, z is the polymer composition in mole % monomer residue
n is molecular weight

Exemplary di-block copolymers:

[DMAEMA]-[B—/—P-/-D]

[PEGMA_{w}]-[B—/—P-/-D]

[PEGMA_{w}-DMAEMA]-[B—/—P-/-D]

[PEGMA_{w}-MAA(NHS)]-[B—/—P-/-D]

[DMAEMA-/-MAA(NHS)]-[B—/—P-/-D]

[HPMA-/-PDSM]-[B—/—P-/-D]

where:
B is butyl methacrylate
P is propyl acrylic acid
D is DMAEMA is dimethylaminoethyl methacrylate
PEGMA is polyethyleneglycol methacrylate where, for example, w=4-5 or 7-8 ethylene oxide units)
MAA(NHS) is methylacrylic acid-N-hydroxy succinimide
HPMA is N-(2-hydroxypropyl)methacrylamide
PDSM is pyridyl disulfide methacrylate

These polymers represent structures where the composition of the first block of the polymer or copolymer is varied or chemically treated in order to create polymers where the first block is neutral (e.g., PEGMA), cationic (DMAEMA), anionic (PEGMA-NHS, where the NHS is hydrolyzed to the acid), ampholytic (DMAEMA-NHS, where the NHS is hydrolyzed to the acid), or zwitterionic (for example, poly[2-methacryloyloxy-2′trimethylammoniumethyl phosphate]). In addition, the [PEGMA-PDSM]-[B—P-D] polymer contains a pyridyl disulfide functionality in the first block that can be reacted with a thiolated siRNA to form a polymer-siRNA conjugate.

### Example 1.1

### General Synthetic Procedures for Preparation of Block Copolymers by RAFT

A. RAFT Chain Transfer Agent.

The synthesis of the chain transfer agent (CTA), 4-Cyano-4-(ethylsulfanylthiocarbonyl) sulfanylpentanoic acid (ECT), utilized for the following RAFT polymerizations, was adapted from a procedure by Moad et al., Polymer, 2005, 46(19): 8458-68. Briefly, ethane thiol (4.72 g, 76 mmol) was added over 10 minutes to a stirred suspension of sodium hydride (60% in oil) (3.15 g, 79 mmol) in diethyl ether (150 ml) at 0° C. The solution was then allowed to stir for 10 minutes prior to the addition of carbon disulfide (6.0 g, 79 mmol). Crude sodium S-ethyl trithiocarbonate (7.85 g, 0.049 mol) was collected by filtration, suspended in diethyl ether (100 mL), and reacted with Iodine (6.3 g, 0.025 mol). After 1 hour the solution was filtered, washed with aqueous sodium thiosulfate, and dried over sodium sulfate. The crude bis(ethylsulfanylthiocarbonyl) disulfide was then isolated by rotary evaporation. A solution of bis-(ethylsulfanylthiocarbonyl) disulfide (1.37 g, 0.005 mol) and 4,4′-azobis(4-cyanopentanoic acid) (2.10 g, 0.0075 mol) in ethyl acetate (50 mL) was heated at reflux for 18 h. Following rotary evaporation of the solvent, the crude 4-Cyano-4(ethylsulfanylthiocarbonyl) sulfanylpentanoic acid (ECT) was isolated by column chromatography using silica gel as the stationary phase and 50:50 ethyl acetate hexane as the eluent.

B. Poly(N,N-dimethylaminoethyl methacrylate) macro chain transfer agent (polyDMAEMA macroCTA).

The RAFT polymerization of DMAEMA was conducted in DMF at 30° C. under a nitrogen atmosphere for 18 hours using ECT and 2,2′-Azobis(4-methoxy-2,4-dimethyl valeronitrile) (V-70) (Wako chemicals) as the radical initiator. The initial monomer to CTA ratio ([CTA]₀/[M]₀ was such that the theoretical Mₙ at 100% conversion was 10,000 (g/mol). The initial CTA to initiator ratio ([CTA]ₒ/[I]ₒ) was 10 to 1. The resultant polyDMAEMA macro chain transfer agent was isolated by precipitation into 50:50 v:v diethyl ether/pentane. The resultant polymer was redissolved in acetone and subsequently precipitated into pentane (×3) and dried overnight in vacuo.

C. Block Copolymerization of DMAEMA, PAA, and BMA from a Poly(DMAMEA) MacroCTA.

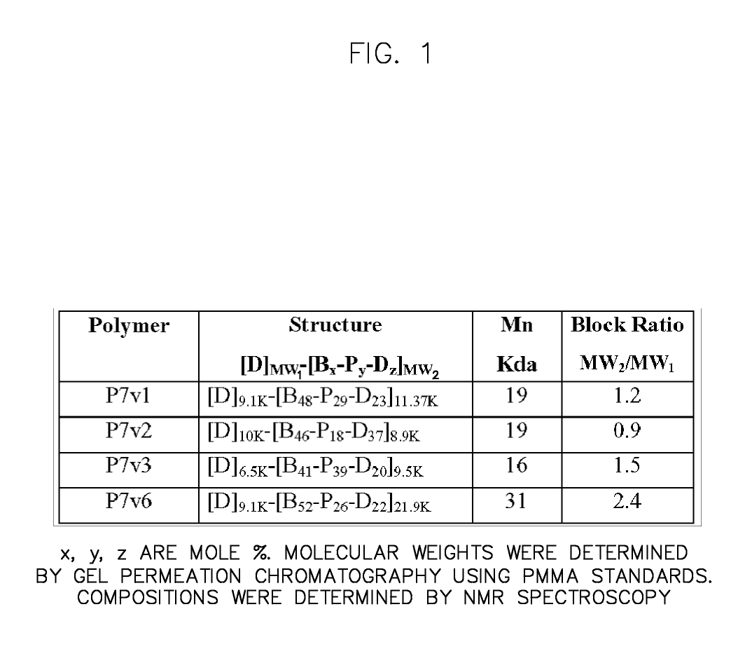

The desired stoichiometric quantities of DMAEMA, PAA, and BMA were added to poly(DMAEMA) macroCTA dissolved in N,N-dimethylformamide (25 wt % monomer and macroCTA to solvent). For all polymerizations [M]ₒ/[CTA]ₒ and [CTA]ₒ/[I]ₒ were 250:1 and 10:1 respectively. Following the addition of V70 the solutions were purged with nitrogen for 30 min and allowed to react at 30° C. for 18 h. The resultant diblock copolymers were isolated by precipitation into 50:50 v:v diethyl ether/pentane. The precipitated polymers were then redissolved in acetone and subsequently precipitated into pentane (×3) and dried overnight in vacuo. Gel permeation chromatography (GPC) was used to determine molecular weights and polydispersities (PDI, M_{w}/Mₙ) of both the poly(DMAEMA) macroCTA and diblock copolymer samples in DMF with respect to polymethyl methacrylate standards (SEC Tosoh TSK-GEL R-3000 and R-4000 columns (Tosoh Bioscience, Montgomeryville, Pa.) connected in series to a Viscotek GPCmax VE2001 and refractometer VE3580 (Viscotek, Houston, Tex.). HPLC-grade DMF containing 1.0 wt % LiBr was used as the mobile phase. FIG. 1 summarizes the molecular weights and compositions of some of the RAFT synthesized polymers.

### Example 1.2

### Preparation of Second Block (B1-B2-B3) Copolymerization of DMAEMA, PAA, and BMA from a Poly(PEGMA) MacroCTA

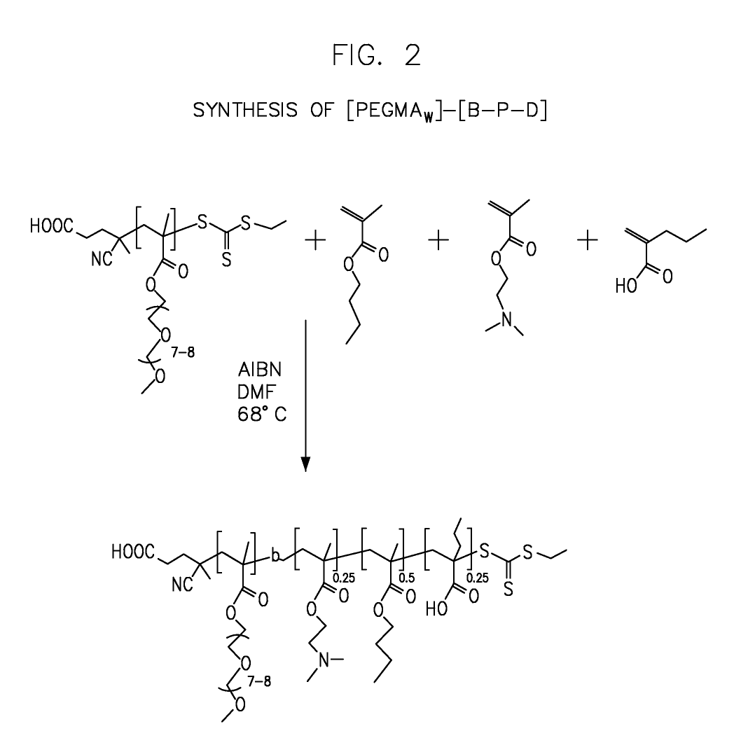

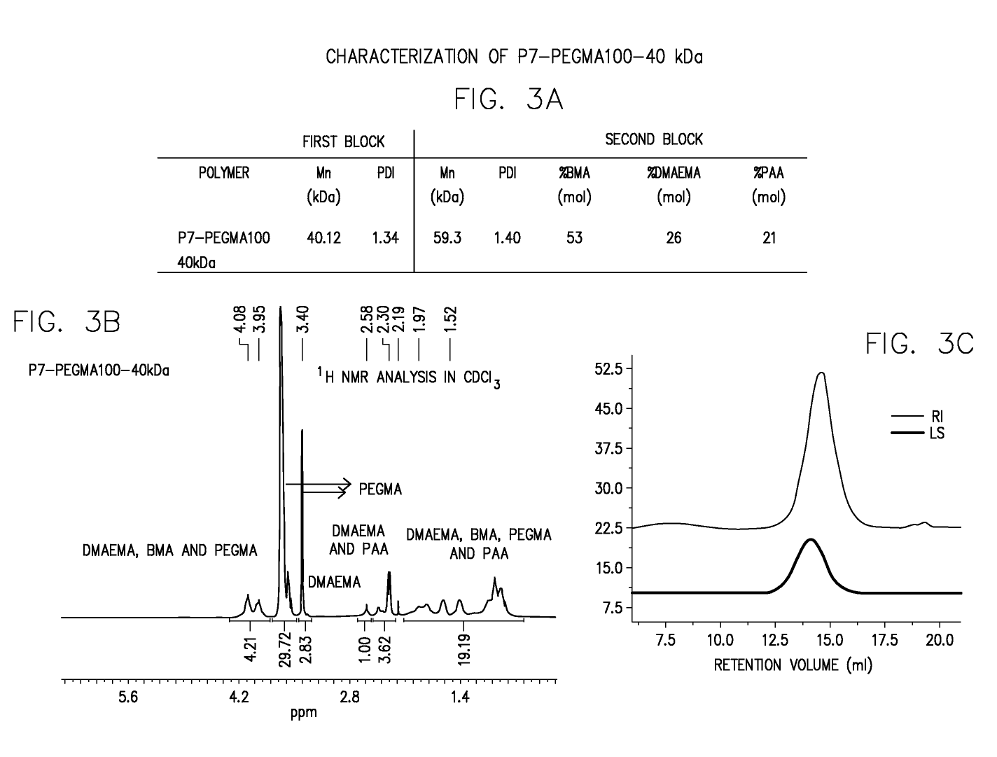

The desired stoichiometric quantities of DMAEMA, PAA, and BMA were added to poly(PEGMA) macroCTA dissolved in N,N-dimethylformamide (25 wt % monomer and macroCTA to solvent). For all polymerizations [M]ₒ/[CTA]ₒ and [CTA]ₒ/[I]ₒ were 250:1 and 10:1 respectively. Following the addition of AIBN the solutions were purged with nitrogen for 30 min and allowed to react at 68° C. for 6-12 h (FIG. 2). The resulting diblock copolymers were isolated by precipitation into 50:50 v:v diethyl ether/pentane. The precipitated polymers were then redissolved in acetone and subsequently precipitated into pentane (×3) and dried overnight in vacuo. Gel permeation chromatography (GPC) was used to determine molecular weights and polydispersities (PDI, M_{w}/Mₙ) of both the poly(PEGMA) macroCTA and diblock copolymer samples in DMF using a Viscotek GPCmax VE2001 and refractometer VE3580 (Viscotek, Houston, Tex.). HPLC-grade DMF containing 1.0 wt % LiBr was used as the mobile phase. NMR spectroscopy in CDCl₃ was used to confirm the polymer structure and calculate the composition of the 2^{nd} block. FIG. 2 summarizes the synthesis of [PEGMA_{w}]-[B—P-D] polymer where w=7-8 and FIGS. 3A, 3B and 3C summarize the characterization of [PEGMA_{w}]-[B—P-D] polymer where w=7-8.

### Example 1.3

### Preparation and Characterization of PEGMA-DMAEMA Co-Polymers

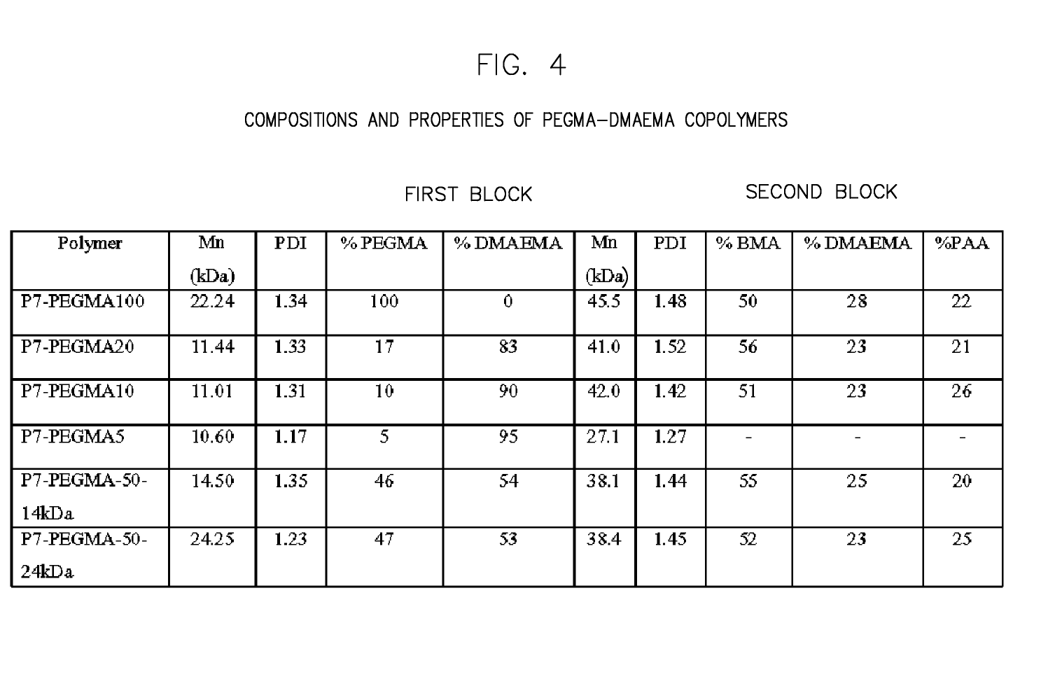

Polymer synthesis was carried out using a procedure similar to that described in Examples 1.1 and 1.2. The ratio of the PEGM and DMAEMA in the first block was varied by using different feed ratios of the individual monomers to create the co-polymers described in FIG. 4.

### Example 1.4

### Preparation and Characterization of PEGMA-MAA(NHS) Co-Polymers

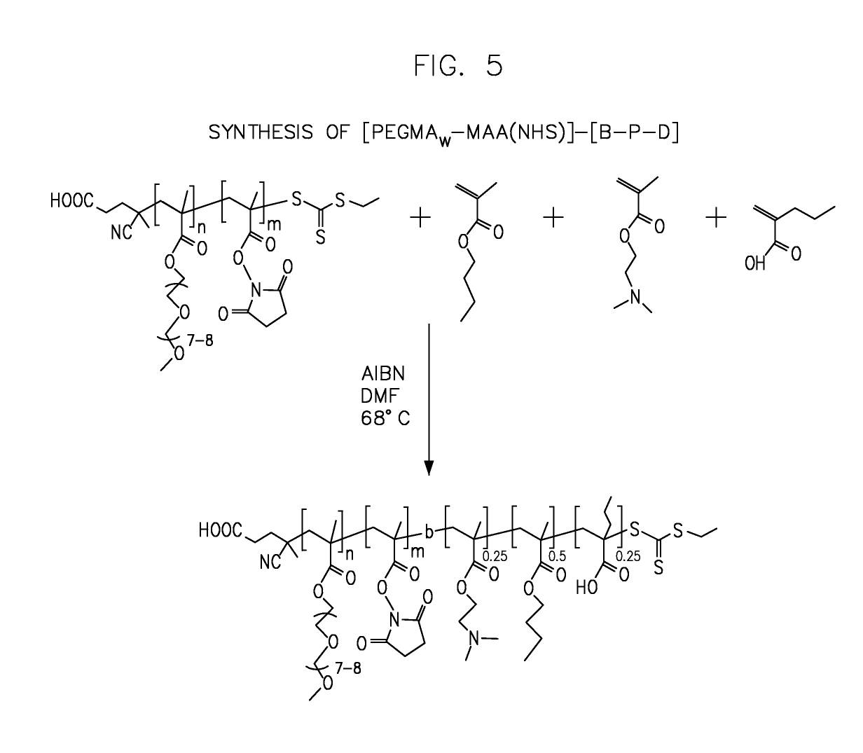

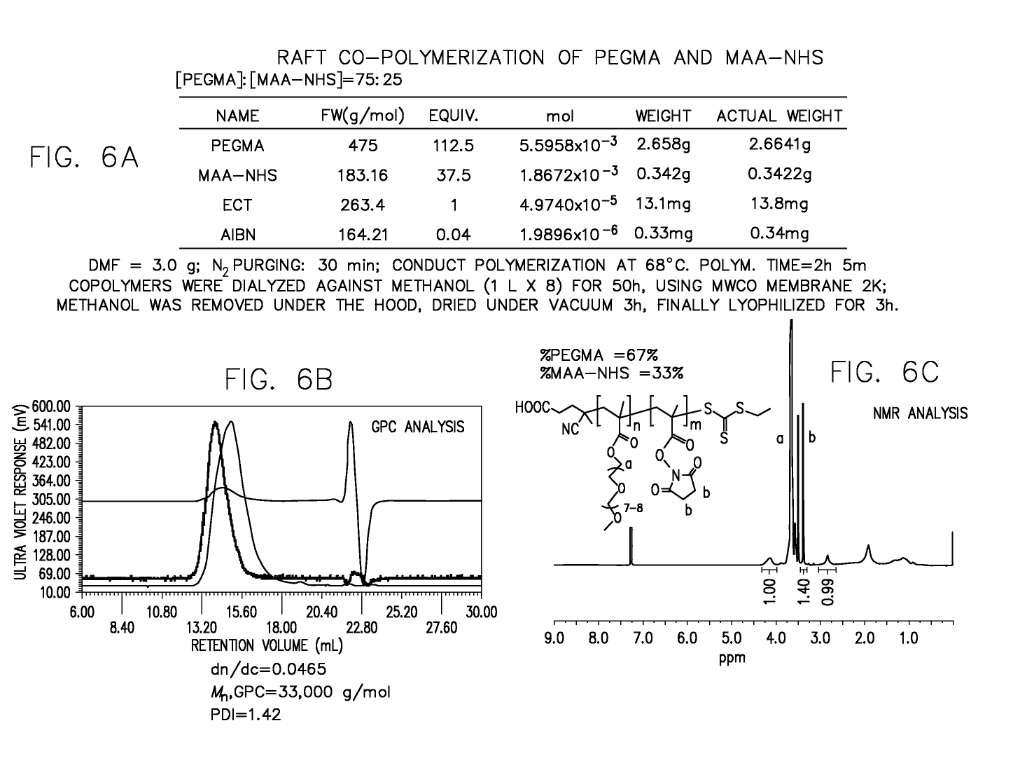

Polymer synthesis was performed as described in Examples 1.1 and 1.2 (and summarized in FIG. 5), using monomer feed ratios to obtain the desired composition of the 1^{st} block copolymer. FIGS. 6A, 6B and 6C summarize the synthesis and characterization of [PEGMA_{w}-MAA(NHS)]-[B—P-D] polymer where the co-polymer ratio of monomers in the 1^{st} block is 75:25. NHS containing polymers can be incubated in aqueous buffer (phosphate or bicarbonate) at pH between 7.4 and 8.5 for 1-4 hrs at room temperature or 37° C. to generate the hydrolyzed (acidic) form.

### Example 1.5

### Preparation and Characterization of DMAEMA-MAA(NHS) Co-Polymers

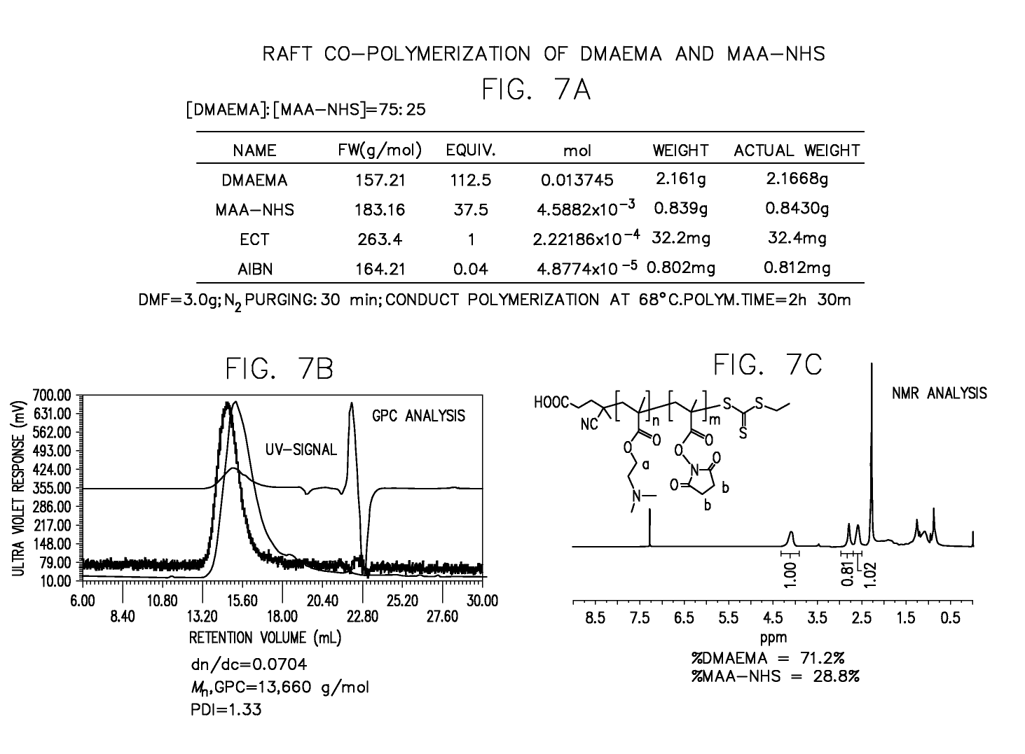

Polymer synthesis was performed as described in Examples 1.1 and 1.2, using monomer feed ratios to obtain the desired composition of the 1^{st} block copolymer. FIGS. 7A, 7B and 7C summarize the synthesis and characterization of [DMAEMA-MAA(NHS)]-[B—P-D] polymer where the co-polymer ratio of monomers in the 1^{st} block is 70:30. NHS containing polymers can be incubated in aqueous buffer (phosphate or bicarbonate) at pH between 7.4 and 8.5 for 1-4 hrs at room temperature or 37° C. to generate the hydrolyzed (acidic) form.

### Example 2

### Preparation and Characterization of HPMA-PDS(RNA) Co-Polymer Conjugates for siRNA Drug Delivery

A. Synthesis of Pyridyl Disulfide Methacrylate Monomer (PDSMA).

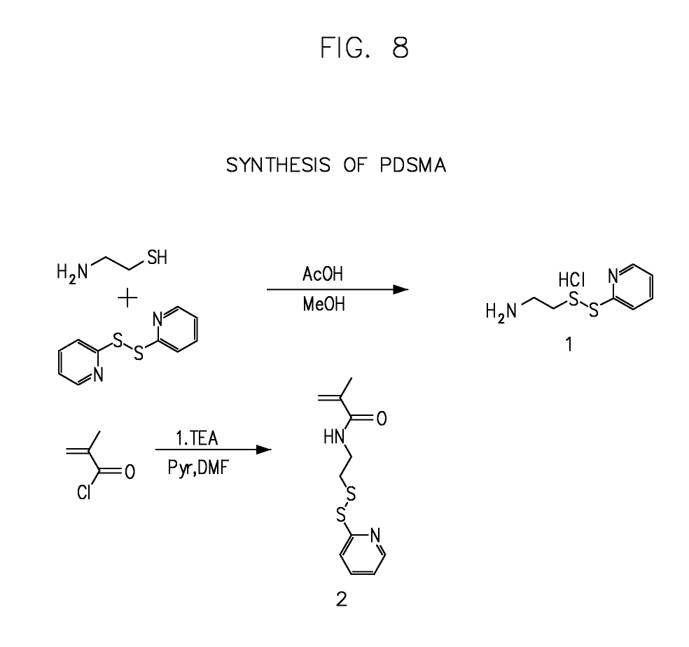

The synthesis scheme for PDSMA is summarized in FIG. 8. Aldrithiol-2™ (5 g, 22.59 mmol) was dissolved in 40 ml of methanol and 1.8 ml of AcOH. The solution was added as a solution of 2-aminoethanethiol.HCl (1.28 g, 11.30 mmol) in 20 ml methanol over 30 min. The reaction was stirred under N₂ for 48 h at R.T. After evaporation of solvents, the residual oil was washed twice with 40 ml of diethyl ether. The crude compound was dissolved in 10 ml of methanol and the product was precipitated twice with 50 ml of diethyl ether to get the desired compound 1 as slight yellow solid. Yield: 95%.

Pyridine dithioethylamine (1, 6.7 g, 30.07 mmol) and triethylamine (4.23 ml, 30.37 mmol) were dissolved in DMF (25 ml) and pyridine (25 ml) and methacryloyl chloride (3.33 ml, 33.08 mmol) was added slowly via syringe at 0 C. The reaction mixture was stirred for 2 h at R.T. After reaction, the reaction was quenched by sat. NaHCO₃ (350 ml) and extracted by ethyl acetate (350 ml). The combined organic layer was further washed by 10% HCl (100 ml, 1 time) and pure water (100 ml, 2 times) and dried by MaSO₄. The pure product was purified by column chromatography (EA/Hex: 1/10 to 2/1) as yellow syrup. R_{f}=0.28 (EA/Hex=1/1). Yield: 55%.

B. HPMA-PDSMA Co-Polymer Synthesis

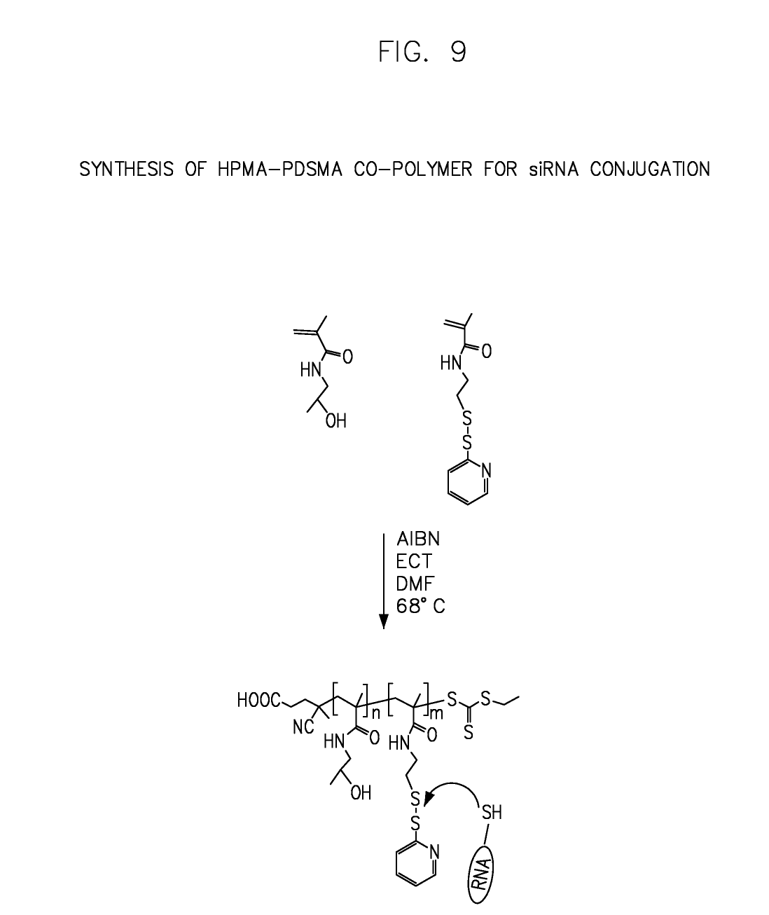

The RAFT polymerization of N-(2-hydroxypropyl)methacrylamide (HPMA) and pyridyl disulfide methacrylate (typically at a 70:30 monomer ratio) is conducted in DMF (50 weight percent monomer:solvent) at 68° C. under a nitrogen atmosphere for 8 hours using 2,2′-azo-bis-isobutyrylnitrile (AIBN) as the free radical initiator (FIG. 9). The molar ratio of CTA to AIBN is 10 to 1 and the monomer to CTA ratio is set so that a molecular weight of 25,000 g/mol would be achieved if at 100% conversion. The poly(HPMA-PDS) macro-CTA was isolated by repeated precipitation into diethyl ether from methanol.

The macro-CTA is dried under vacuum for 24 hours and then used for block copolymerization of dimethylaminoethyl methacrylate (DMAEMA), propylacrylic acid (PAA), and butyl methacrylate (BMA). Equimolar quantities of DMAEMA, PAA, and BMA ([M]ₒ/[CTA]ₒ=250) are added to the HPMA-PDS macroCTA dissolved in N,N-dimethylformamide (25 wt % monomer and macroCTA to solvent). The radical initiator AIBN is added with a CTA to initiator ratio of 10 to 1. The polymerization is allowed to proceed under a nitrogen atmosphere for 8 hours at 68° C. Afterwards, the resultant diblock polymer is isolated by precipitation 4 times into 50:50 diethyl ether/pentane, redissolving in ethanol between precipitations. The product is then washed 1 time with diethyl ether and dried overnight in vacuo.

C. siRNA Conjugation to HPMA-PDSMA Co-Polymer

Thiolated siRNA was obtained commercially (Agilent, Boulder, Colo.) as a duplex RNA with a disulfide modified 5′-sense strand. The free thiol form for conjugation is prepared by dissolving the lyophilized compound in water and treated for 1 hour with the disulfide reducing agent TCEP immobilized within an agarose gel. The reduced RNA (400 μM) was then reacted for 24 hours with the pyridyl disulfide-functionalized polymer in phosphate buffer (pH 7) containing 5 mM ethylenediaminetetraacetic acid (EDTA) (FIG. 8).

The reaction of the pyridyl disulfide polymer with the RNA thiol creates 2-pyridinethione, which can be spectrophotometrically measured to characterize conjugation efficiency. To further validate disulfide exchange, the conjugates are run on an SDS-PAGE 16.5% tricine gel. In parallel, aliquots of the conjugation reactions are treated with immobilized TCEP prior to SDS-PAGE to verify release of the RNA from the polymer in a reducing environment. Conjugation reactions are conducted at polymer/RNA stoichiometries of 1, 2, and 5. UV spectrophotometric absorbance measurements at 343 nm for 2-pyridinethione release are used to measure conjugation efficiencies.

### Example 3

### Synthesis of Polymers with Cell Targeting Agents: Click Reaction of Azido-Terminated Polymer with Propargyl Folate

A combination of controlled radical polymerization and azide-alkyne click chemistry is used to prepare block copolymer micelles conjugated with biological ligands (for example, folate) with potential for active targeting of specific tissues/cells containing the specific receptor of interest (for example, folate). Block copolymers are synthesized by reversible addition-fragmentation chain transfer (RAFT) polymerization as described in Example 1, except that an azido chain transfer agent (CTA) is used. The azido terminus of the polymer is then reacted with the alkyne derivative of the targeting agent (for example, folate) to produce the polymer containing the targeting agent.

Synthesis of the RAFT Agent.

The RAFT chain transfer agent (CTA) 2-dodecylsulfanylthiocarbonylsulfanyl-2-methyl-propionic acid 3-azidopropyl ester (C12-CTAN3) is prepared as follows:

Synthesis of 3-Azidopropanol. 3-Chloro-1-propanol (5.0 g, 53 mmol, 1.0 equiv) and sodium azide (8.59 g, 132 mmol, 2.5 equiv) are reacted in DMF (26.5 mL) at 100° C. for 48 h. The reaction mixture is cooled to room temperature, poured into ethyl ether (200 mL), and extracted with a saturated aqueous NaCl solution (500 mL). The organic layer is separated, dried over MgSO4, and filtered. The supernatant is concentrated to obtain the product (5.1 g, 95% yield).

Synthesis of 2-dodecylsulfanylthiocarbonylsulfanyl-2-methylpropionic acid chloride (DMP-C1). 2-dodecylsulfanylthiocarbonylsulfanyl-2-methyl-propionic acid (DMP, Noveon>95%) (1.0 g, 2.7 mmol, 1.0 equiv) is dissolved in methylene chloride (15 mL) in a 50 mL round-bottom flask, and the solution is cooled to approximately 0° C. Oxalyl chloride (0.417 g, 3.3 mmol, 1.2 equiv) is added slowly under a nitrogen atmosphere, and the solution is allowed to reach room temperature and stirred for a total of 3 h. The resulting solution is concentrated under reduced pressure to yield the acid chloride product (1.0 g, 99% yield).

Synthesis of 2-dodecylsulfanylthiocarbonylsulfanyl-2-methylpropionic acid 3-azidopropyl ester. 3-Azidopropanol (265 mg, 2.62 mmol, 1.0 equiv) is dissolved in methylene chloride (5 mL) in a 50 mL round-bottom flask, and the solution is cooled to approximately 0° C. A solution of triethylamine (0.73 mL) in methylene chloride (5 mL) is added dropwise over 10 min. A solution of DMP-C1 (1.0 g, 2.6 mmol) in methylene chloride (5 mL) is added dropwise, and the solution is allowed to reach room temperature while stirring for 3 h. The solution is concentrated under reduced pressure, diluted with ethyl ether (100 mL), and washed with saturated aqueous sodium bicarbonate solution (50 mL), water (50 mL), and saturated NaCl solution (50 mL), successively. The organic layer is separated, dried over MgSO4 (1.0 g), and filtered. The supernatant is concentrated under reduced pressure to yield the product (1.05 g, 90% yield) as a residual oil.

Synthesis of Propargyl Folate.

Folic acid (1.0 g, 0.0022 mol) is dissolved in DMF (10 mL) and cooled in a water/ice bath. N-Hydroxysuccinimide (260 mg, 0.0025 mol) and EDC (440 mg, 0.0025 mol) are added, and the resulting mixture is stirred in an ice bath for 30 min to give a white precipitate. A solution of propargylamine (124 mg, 2.25 mmol) in DMF (5.0 mL) is added, and the resulting mixture is allowed to warm to room temperature and stirred for 24 h. The reaction mixture is poured into water (100 mL) and stirred for 30 min to form a precipitate. The orange-yellow precipitate is filtered, washed with acetone, and dried under vacuum for 6 h to yield 1.01 g of product (93% yield).

Click reaction of azido-terminated polymers with propargyl folate.

The azido-terminated polymer is reacted with propargyl folate by the following example procedure. A solution of N3-α-[Dₛ-Xₜ]_{b}—[Bₓ—P_{y}-D_{z}]ₐ-ω (0.0800 mmol) in DMF (7 mL), and pentamethyldiethylenetriamine (PMDETA, Aldrich, 99%), (8.7 mg, 0.050 mmol) is purged with nitrogen for 60 min and transferred via syringe to a vial equipped with a magnetic stir bar containing CuBr (7.2 mg, 0.050 mmol) and propargyl folate (42 mg, 0.088 mmol) under a nitrogen atmosphere. The reaction mixture is stirred at 26° C. for 22 h in the absence of oxygen. The reaction mixture is exposed to air, and the solution is passed through a column of neutral alumina. DMF is removed under vacuum, and the product is precipitated into hexanes. The resulting folate-terminated block copolymer folate-α-[Dₛ-Xₜ]_{b}—[Bₓ—P_{y}-D]ₐ-ω is dissolved in THF and filtered to remove excess propargyl folate. THF is removed, and then the polymer is dissolved in deionized (DI) water and dialyzed for 6 h using a membrane with a molecular weight cutoff of 1000 Da. The polymer is isolated by lyophilization.

### Example 4

### NMR Spectroscopy of Block Copolymer PRx0729v6

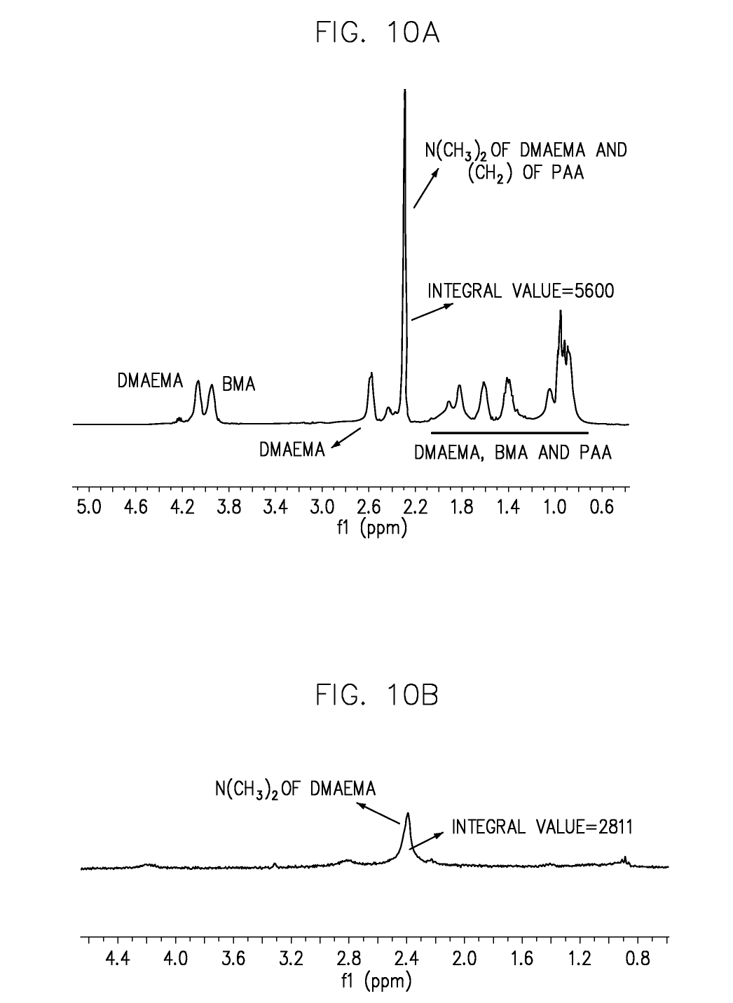

### FIG. 10

This example provides evidence, using NMR spectroscopy, that polymer PRx0729v6 forms a micelle-like structure in aqueous solution.

¹H NMR spectra were recorded on Bruker AV301 in deuterated chloroform (CDCl₃) and deuterated water (D₂O) at 25° C. A deuterium lock (CDCl₃, D₂O) was used, and chemical shifts were determined in ppm from tetramethylsilane (for CDCl₃) and 3-(trimethylsilyl)propionic-2,2,3,3-d4 acid, sodium salt (for D₂O). Polymer concentration was 6 mg/mL.

NMR spectroscopy of the synthesized polymer, using polymer PRx0729v6 as an example, in aqueous buffer provided evidence that the diblock polymers of the present invention form micelles in aqueous solution. Formation of micelles results in the formation of a shielded viscous internal core that restricts the motion of the protons forming the core segments and prevents deuterium exchange between the solvent and the protons of the core. This is reflected by a significance suppression or disappearance of the ¹H NMR signals of the corresponding protons. We used this inherent property of solution NMR spectroscopy to show that the hydrophobic block of the core of the micelle is effectively shielded. If micelles are formed in aqueous media, a disappearance of the signals due to the protons of the hydrophobic copolymer block should occur.

FIG. 10 shows the ¹H NMR experiments of polymer PRx0729v6 in CDCl₃ (organic solvent) and D₂O (aqueous solvent). The ¹H NMR spectrum of polymer in CDCl₃ at room temperature (FIG. 10A) shows the signals attributed to all polymer protons indicating that the polymer chains remain dispersed (non-aggregated) in CDCl₃ and preserve their motion so their protons can exchange with the solvent. This indicates that stable micelles with shielded cores are not formed from PRx0729v6 in organic solvent. FIG. 10B shows the ¹H NMR spectra of PRx0729v6 in D₂O. The signals representing the protons of the hydrophobic block (BMA, PAA, DMAEMA) disappear from the spectrum. This indicates that stable micelles with shielded cores are formed from PRx0729v6 in aqueous solution. Moreover, in the same spectrum, the signal attributed to the resonance of the protons of the two methyl groups of the DMAEMA (2.28 ppm) undergoes a significant suppression, implying that only the first poly DMAEMA block constituting the shell is exposed to water, i.e., mainly the charged group of DMAEMA. A simple calculation indicates that the integrated percentage of PAA, DMAEMA of the hydrophobic block (2900) subtracted from the signal in CDCl₃ (5600) gives the approximate value for the same signal in D₂O (2811), consistent with this conclusion.

Taken together, the results of ¹H NMR experiments indicate that polymer PRx0729v6 forms micelles with an ordered core-shell structure where the first block polyDMAEMA forms a hydrated outer shell surrounding a core composed of hydrophobic units (BMA) and electrostatically stabilizing units of opposite charge (PAA, DMAEMA).

### Example 5

### Polymer PRx0729v6 Particle Stability in Organic Solvents

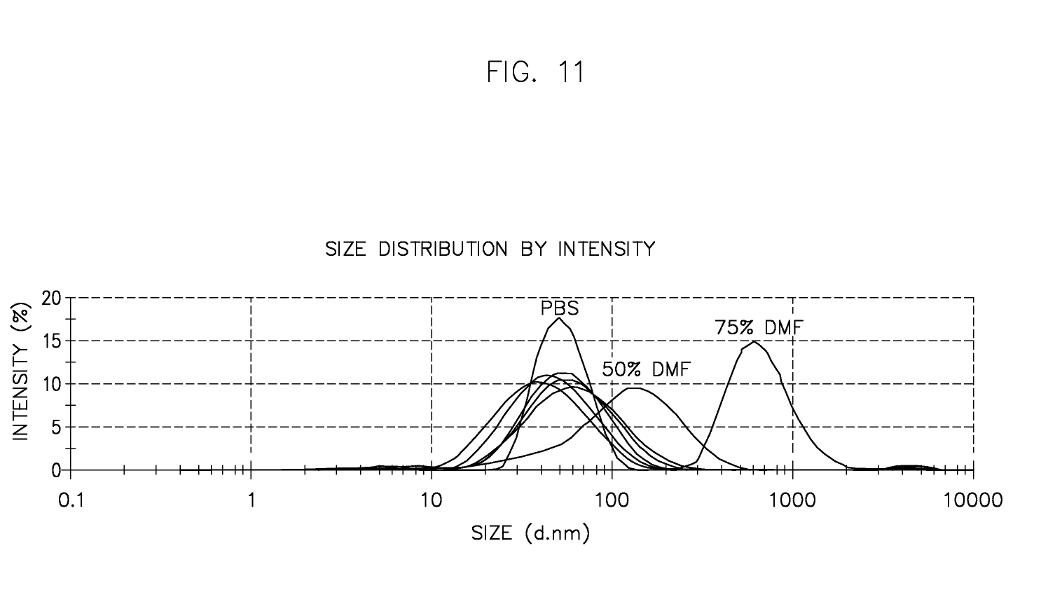

### FIG. 11

This example demonstrates that the micelle structure of polymer PRx0729v6 is dissociated in organic solvents, consistent with the hydrophobic nature of the micelle core.

Polymer PRx0729v6 was dissolved in various organic solvents at a concentration of 1 mg/mL and particle size was measured by dynamic light scattering. FIG. 11 shows that increasing concentration of dimethylformamide (DMF) results in micelle dissociation to aggregated chains.

### Example 6

### Transmission Electron Microscopy (TEM) Analysis of Polymer PRx0729v6

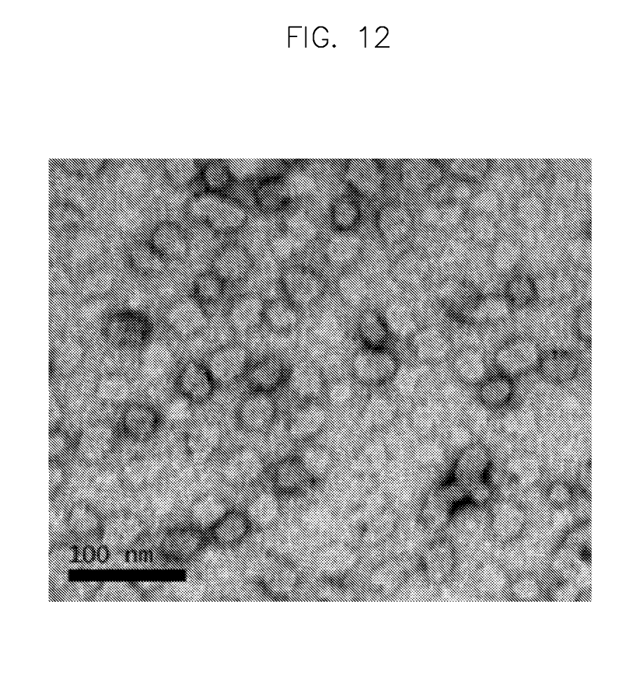

### FIG. 12

This example provides evidence, using electron spectroscopy, that the polymer PRx0729v6 forms spherical micelle-like particles.

A 0.5 mg/mL solution of polymer PRx0729v6 in PBS was applied to a carbon coated copper grid for 30 minutes. The grid was fixed in Karnovsky's solution and washed in cacodylate buffer once and then in water 8 times. The grid was stained with a 6% solution of uranyl acetate for 15 minutes and then dried until analysis. Transmission electron microscopy (TEM) was carried out on a JEOL microscope. FIG. 12 shows a typical electron micrograph of polymer PRx0729v6 demonstrating spherical particles with approximate dimensions similar to those determined in solution by dynamic light scattering.

### Example 7

### Effect of pH on Polymer Structure

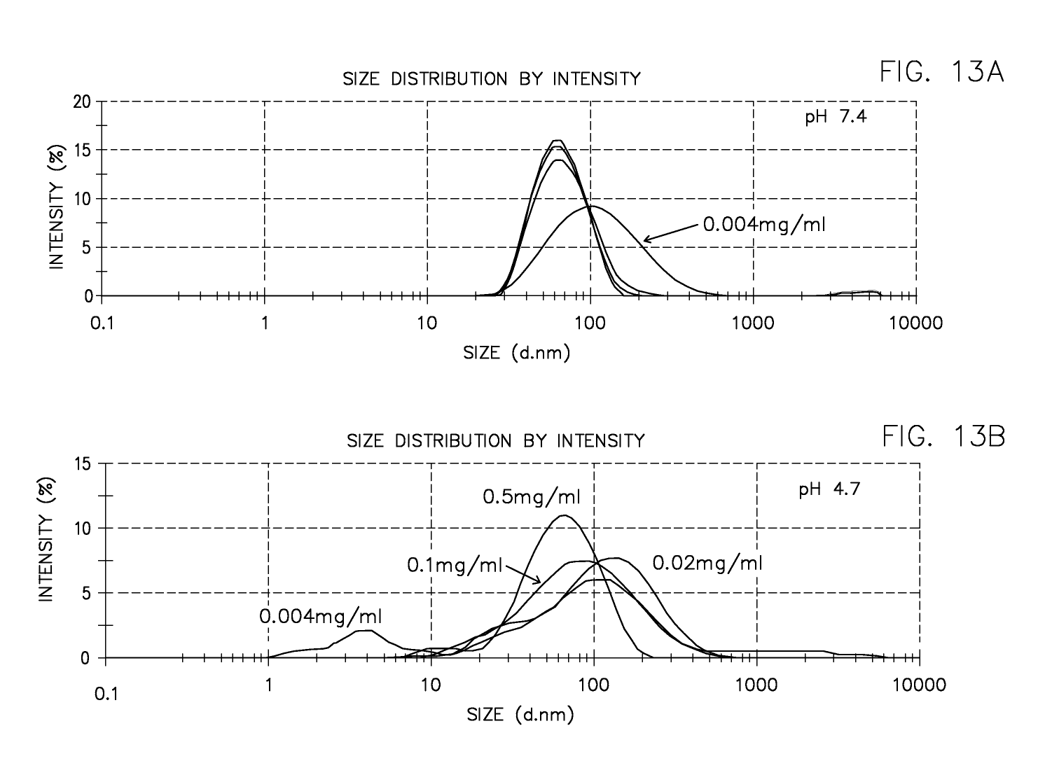

### FIG. 13

This example demonstrates that the micelle structure of polymer PRx0729v6.2 is dissociated upon lowering the pH from 7.4 to 4.7.

Particle Size of polymer PRx0729v6.2 was measured by dynamic light scattering at pH 7.4 and a series of acidic pH values down to pH4.7 in PBS at 5-fold serial dilutions from 0.5 mg/mL-0.004 mg/mL. FIG. 13A shows that at pH 7.4, the polymer is stable to dilution down to 4 μg/mL where it begins to dissociate to a form that produces aggregates. FIG. 13B shows that at increasing acidic pH values down to pH 4.7 the polymer dissociation from a micelle structure is enhanced, that is, occurs at higher polymer concentrations, and produces increasing levels of polymer monomers from 1-8 nm in size.

### Example 8

### Critical Micelle Concentration (CMC) of Polymer PRx0729v6

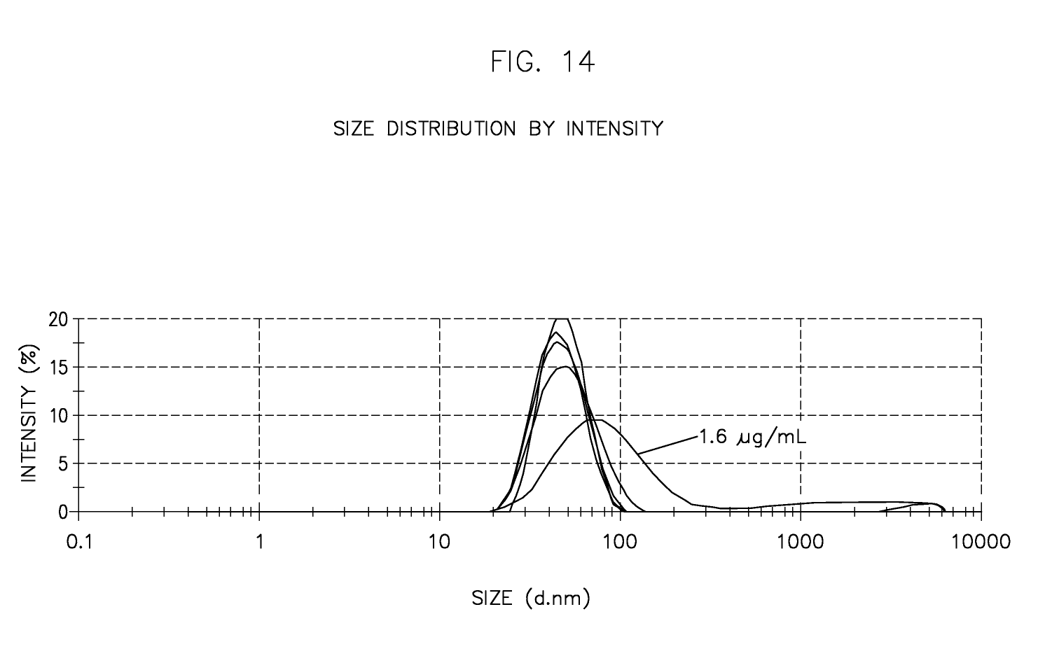

### FIG. 14

The following example demonstrates that micelles formed by polymer PRx0729v6 are stable to 100-fold dilution.

Particle sizes of polymer PRx0729v6 in PBS buffer pH 7.4 at a concentration of 1 mg/mL±0.5 M NaCl. Particle size was measured by dynamic light scattering over a 5-fold range of serial dilutions from 1 mg/mL to 1.6 μg/mL with PBS±0.5 M NaCl. FIG. 14 shows that a particle size of about 45 nm is stable down to a concentration of about 10 μg/mL. Polymer PRx0729v6 appears to be unstable below about 5 μg/mL (the CMC) where individual polymer chains dissociate and form non-specific aggregates.

### Example 9

### Preparation of Heterogeneous (Mixed) Polymer Micelles

A heterogeneous (mixed) polymer micelle comprises two or more compositionally distinct polymers. Each of the two or more compositionally distinct polymers (e.g., Polymer A and Polymer B) can be block copolymers comprising a hydrophilic block and a hydrophobic block.

The heterogeneous micelle can be formed by providing a first polymer and a second polymer compositionally distinct from the first polymer in a first denaturing medium to form a heterogeneous mixture of the first polymer and the second polymer. The heterogeneous mixture is exposed to a second aqueous medium, and the hydrophobic block of the first polymer is allowed to associate with the hydrophobic block of the second polymer in the aqueous medium to assemble into and form a heterogeneous micelle comprising the first polymer and the second polymer.

A polynucleotide can be associated (e.g., ionically or covalently coupled) with at least one of the first polymer, the second polymer or a heterogeneous micelle.

As a non-limiting example, a first polymer comprising block copolymer #1 is prepared by RAFT polymerization as described in Example 1. A second polymer comprising Block copolymer #2 is similarly prepared with a different hydrophilic block and the same hydrophobic block. For example, the (polyDMAEMA) cationic hydrophilic block of block copolymer #1 is instead prepared to have a neutral hydrophilic block, for example, such as a homopolymer block comprising monomeric units having polyethylene glycol oligomers covalently linked to pendant groups thereof (e.g., PEGMA). As another example, a heterogeneous polymer micelle can also be prepared using an alternative second polymer which includes a hydrophilic block comprising a random copolymer of 50% DMAEMA and 50% PEGMA formed by mixing equivalent amounts of the two copolymers in 100% ethanol followed by 20-fold dilution in PBS pH 7.4 or dialysis against PBS pH 7.4. In each case, the general procedure above can be followed to form the heterogeneous micelle.

### Example 10

### siRNA/Polymer Complex Characterization

After verification of complete, serum-stable siRNA complexation via agarose gel retardation, siRNA/polymer complexes were characterized for size and zeta potential using a ZetaPALS detector (Brookhaven Instruments Corporation, Holtsville, N.Y., 15 mW laser, incident beam=676 nm). Briefly, polymer was formulated at 0.1 mg/mL in phosphate buffered saline (PBS, Gibco) and complexes were formed by addition of polymer to GAPDH siRNA (Ambion) at the indicated theoretical charge ratios based on positively charged DMAEMA, which is 50% protonated at pH=7.4 and the negatively-charged siRNA. Correlation functions were collected at a scattering angle of 90°, and particle sizes were calculated using the viscosity and refractive index of water at 25° C. Particle sizes are expressed as effective diameters assuming a log-normal distribution. Average electrophoretic mobilities were measured at 25° C. using the ZetaPALS zeta potential analysis software, and zeta potentials were calculated using the Smoluchowsky model for aqueous suspensions.

### Example 11

### HeLa Cell Culture

HeLas, human cervical carcinoma cells (ATCC CCL-2), were maintained in minimum essential media (MEM) containing L-glutamine (Gibco), 1% penicillin-streptomycin (Gibco), and 10% fetal bovine serum (FBS, Invitrogen) at 37° C. and 5% CO₂.

### Example 12

### pH-Dependent Membrane Disruption of Carriers and siRNA/Polymer Complexes

Hemolysis was used to determine the potential endosomolytic activity of both free polymer and siRNA/polymer conjugates at pH values that mimic endosomal trafficking (extracellular pH=7.4, early endosome pH=6.6, and late endosome pH=5.8). Briefly, whole human blood was collected in vaccutainers containing EDTA. Blood was centrifuged, plasma aspirated, and washed three times in 150 mM NaCl to isolate the red blood cells (RBC). RBC were then resuspended in phosphate buffer (PB) at pH 7.4, pH 6.6, or pH 5.8. Polymers (10 μg/mL) or polymer/siRNA complexes were then incubated with the RBC at the three pH values for 1 hour at 37° C. Intact RBC were then centrifuged and the hemoglobin released into supernatant was measured by absorbance at 541 nm as an indication of pH-dependent RBC membrane lysis.

### Example 13

### Measurement of Carrier-Mediated siRNA Uptake

Intracellular uptake of siRNA/polymer complexes was measured using flow cytometry (Becton Dickinson LSR benchtop analyzer). Helas were seeded at 15,000 cells/cm² and allowed to adhere overnight. FAM (5-carboxyfluorescine) labeled siRNA (Ambion) was complexed with polymer at a theoretical charge ratio of 4:1 for 30 min at room temperature and then added to the plated HeLas at a final siRNA concentration of 25 nM. After incubation with the complexes for 4 h, the cells were trypsinized and resuspended in PBS with 0.5% BSA and 0.01% trypan blue. Trypan blue was utilized as previously described for quenching of extracellular fluorescence and discrimination of complexes that have been endocytosed by cells. 10,000 cells were analyzed per sample and fluorescence gating was determined using samples receiving no treatment and polymer not complexed with FAM labeled siRNA.

### Example 14

### sIRNA/Polymer Complex Cytotoxicity

siRNA/polymer complex cytotoxicity was determined using and lactate dehydrogenase (LDH) cytotoxicity detection kit (Roche). HeLa cells were seeded in 96-well plates at a density of 12,000 cells per well and allowed to adhere overnight. Complexes were formed by addition of polymer (0.1 mg/mL stock solutions) to GAPDH siRNA at theoretical charge ratios of 4:1 and to attain a concentration of 25 nM siRNA/well. Complexes (charge ratio=4:1) were added to wells in triplicate. After cells had been incubated for 24 hours with the polymer complexes, the media was removed and the cells were washed with PBS twice. The cells were then lysed with lysis buffer (100 μL/well, 20 mM Tris-HCl, pH 7.5, 150 mM NaCl, 1 mM Na₂EDTA, 1 mM EGTA, 1% Triton, 2.5 mM sodium pyrophosphate, 1 mM β-glycerophosphate, 1 mM sodium orthovanadate) for 1 hour at 4° C. After mixing by pipetting, 20 μL of the cell lysate was diluted 1:5 in PBS and quantified for lactate dehydrogenase (LDH) by mixing with 100 μL of the LDH substrate solution. After a 10-20 min incubation for color formation, the absorbance was measured at 490 nm with the reference set at 650 nm.

### Example 15

### Evaluation of GAPDH Protein and Gene Knockdown by siRNA/Polymer Complexes

The efficacy of the series of polymers for siRNA delivery was screened using a GAPDH activity assay (Ambion). HeLas (12,000 cells/cm²) were plated in 96-well plates. After 24 h, complexes (charge ratios=4:1) were added to the cells at a final siRNA concentration of 25 nM in the presence of 10% serum. The extent of siRNA-mediated GAPDH protein reduction was assessed 48 h post-transfection. As a positive control, parallel knockdown experiments were run using HiPerFect (Qiagen) following manufacturer's conditions. The remaining GAPDH activity was measured as described by the manufacturer using the kinetic fluorescence increase method over 5 min and was calculated according to the following equation: % remaining expression=Δ_{fluorescence, GAPDH}/Δ_{fluorescence, no treatment}, where Δ_{fluorescence}=fluorescence₅ₘᵢₙ−fluoresecence₁ₘᵢₙ. The transfection procedure did not significantly affect GAPDH expression when a nontargeting sequence of siRNA was used.

After the initial screen to identify the carrier that produced the most robust siRNA-mediated GAPDH knockdown, real time reverse transcription polymerase chain reaction (RT-PCR) was used to directly evaluate siRNA delivery. After 48 hours of incubation with complexes as formed above, cells were rinsed with PBS. Total RNA was isolated using Qiagen's Qiashredder and RNeasy mini kit. Any residual genomic DNA in the samples was digested (RNase-Free DNase Set, Qiagen) and RNA was quantified using the RiboGreen assay (Molecular Probes) based on the manufacturer's instructions.

Reverse transcription was performed using the Omniscript RT kit (Qiagen). A 25 ng total RNA sample was used for cDNA synthesis and PCR was conducted using the ABI Sequence Detection System 7000 using predesigned primer and probe sets (Assays on Demand, Applied Biosystems) for GAPDH and β-acting as the housekeeping gene. Reactions (20 μl total) consisted of 10 μL of 2× Taqman Universal PCR Mastermix, 1 μL of primer/probe, and 2 μL of cDNA, brought up to 20 μL with nuclease-free water (Ambion). The following PCR parameters were utilized: 95° C. for 90 s followed by 45 cycles of 95° C. for 30 s and 55° C. for 60 s. Threshold cycle (C_{T}) analysis was used to quantify GAPDH, normalized to 3-actin and relative to expression of untreated HeLas.

### Example 16

### Dynamic Light Scattering (DLS) Determination of Particle Size of Polymer PRx0729v6 Complexed to siRNA

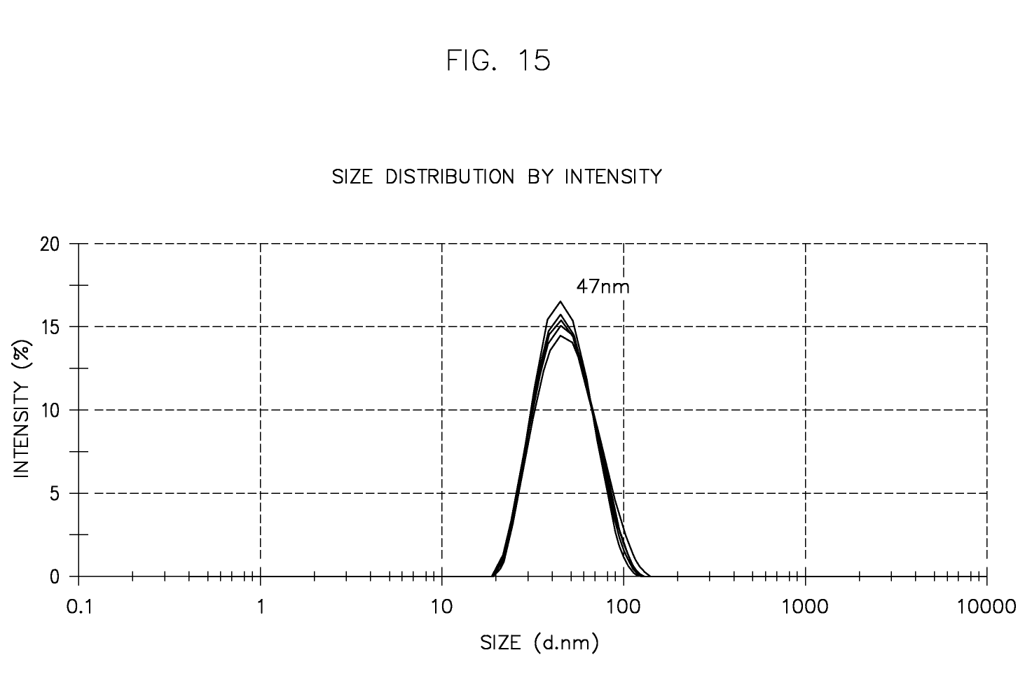

### FIG. 15

The following example demonstrates that polymer PRx0729v6 forms uniform particles 45 nm in size either alone or 47 nm in size following binding to siRNA.

Particle sizes of polymer alone or polymer/siRNA complexes were measured by dynamic light scattering (DLS) using a Malvern Zetasizer Nano ZS. Lyophilized polymer was dissolved in 100% ethanol at 10-50 mg/mL, then diluted 10-fold into phosphate buffer, pH 7.4. Polymers were measured in phosphate buffered saline, pH 7.4 (PBS) at 1 mg/mL for PRx0729v6 alone or at 0.7 mg/mL PRx0729v6 complexed to 1 uM GAPDH-specific 21 mer-siRNA (Ambion), with a theoretical charge ratio of 4:1, positive charges on polymer: negative charges on siRNA. PRx0729v6 alone (45 nm) and PRx0729v6 complexed to siRNA (47 nm) (FIG. 15) show similar particle sizes with a near uniform distribution, PDI<0.1.

### Example 17

### Gel Shift Analysis of Polymer PRx0729v6/siRNA Complexes at Different Charge Ratios

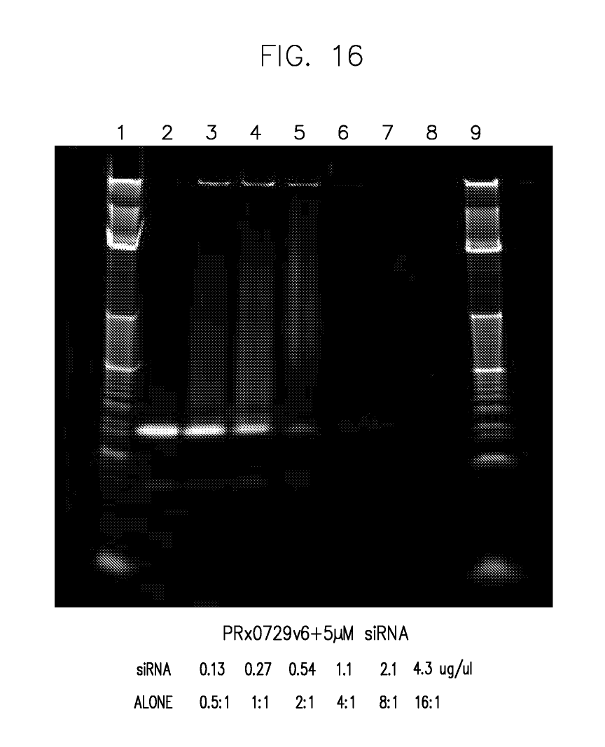

### FIG. 16

The following example demonstrates that polymer PRx0729v6 binds to siRNA at various charge ratios resulting in a complex with reduced electrophoretic mobility.

Polymer siRNA binding was analyzed by gel electrophoresis (FIG. 16) and demonstrates that complete siRNA binding to polymer occurs at a polymer/siRNA charge ratio of 4:1 and higher.

### Example 18

### Conjugation of siRNA with Micelle

A. Conjugation of double-stranded siRNA with thiol-containing block copolymer.

siRNA-pyridyl disulfide was prepared by dissolving amino-siRNA at 10 mg/mL in 50 mM sodium phosphate, 0.15 M NaCl, pH 7.2 or another non-amine buffers, e.g., borate, Hepes, bicarbonate with the pH in the range appropriate for the NHS ester modification (pH 7-9). SPDP was dissolved at a concentration of 6.2 mg/mL in DMSO (20 mM stock solution), and 25 ul of the SPDP stock solution was added to each ml of amino-siRNA to be modified. The solution was mixed and reacted for at least 30 min at room temperature. Longer reaction times (including overnight) did not adversely affect the modification. The modified RNA (pyridyl disulfide) was purified from reaction by-products by dialysis (or gel filtration) using 50 mM sodium phosphate, 0.15 M NaCl, 10 mM EDTA, pH 7.2. The prepared siRNA-pyridyl disulfide was reacted at a 1:5 molar ratio with polymer PRx0729v6 (containing a free thiol at the w-end) in the presence of 10-50 mM EDTA in PBS, pH 7.2. Extent of reaction was monitored spectrophotometrically by release of pyridine-2-thione and by gel electrophoresis.

B. Conjugation of Single Stranded RNA with Polymer Followed by Annealing of the Second Strand.

Single-stranded RNA pyridyl disulfide conjugate was prepared using the procedure of the above example starting with a single stranded amino modified RNA. After the coupling of the RNA pyridyl disulfide with the block copolymer micelle, the complementary RNA strain is added to the reaction mixture, and the two strands are allowed to anneal for 1 hr at a temperature approximately 20° C. below the Tm of the duplex RNA.

### Example 19

### Knock-Down Activity of siRNA

### Micelle Complexes in Cultured Mammalian Cells

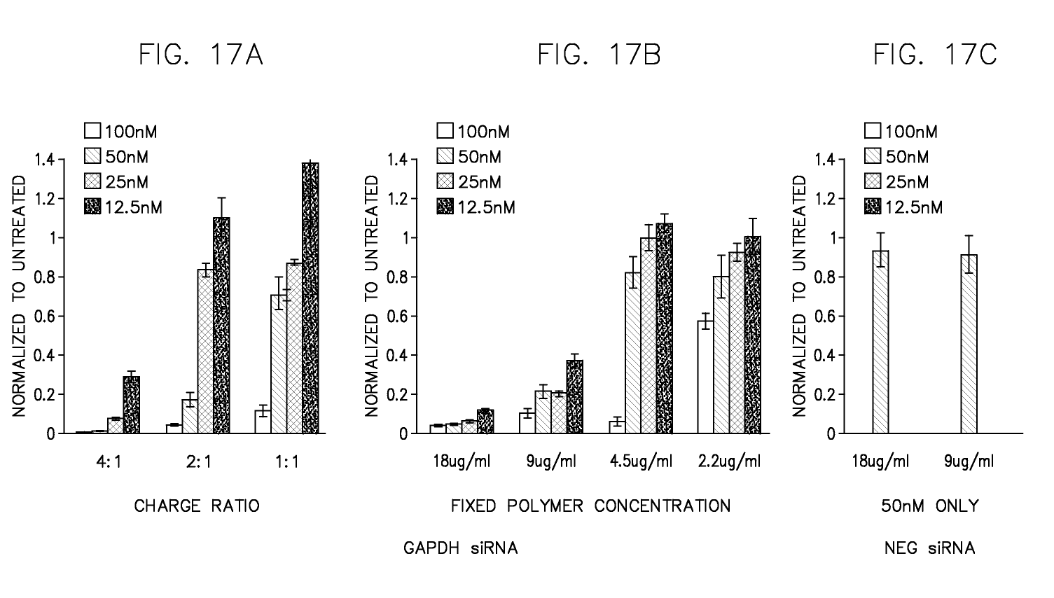

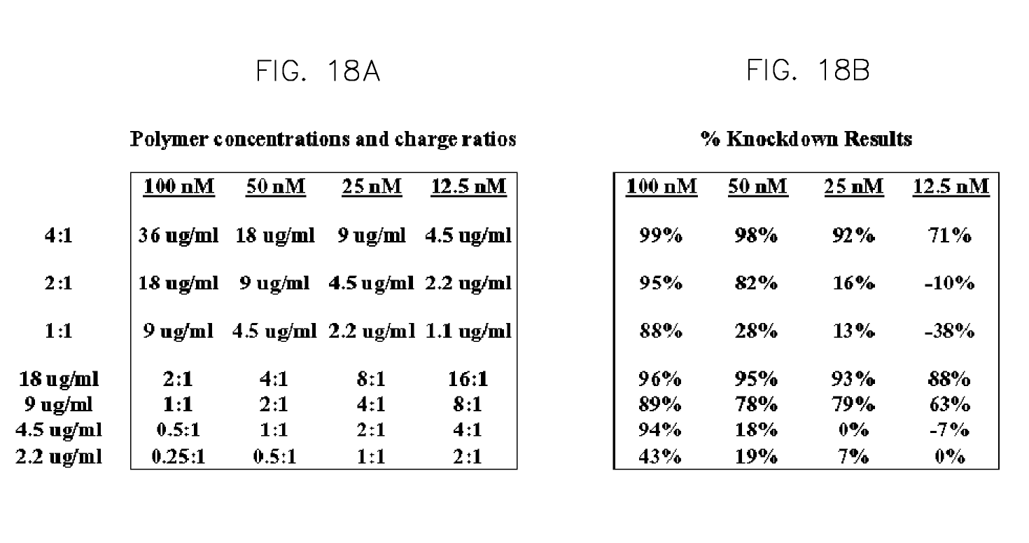

### FIG. 17 and FIG. 18

Knock-down (KD) activity of siRNA/polymer PRx0729v6 complexes was assayed in 96-well format by measuring specific gene expression after 24 hours of treatment with PRx0729v6:siRNA complexes. Polymer and GAPDH targeting siRNA or negative control siRNA (Ambion) were mixed in 25 uL to obtain various charge ratios and concentrations at 5-fold over final transfection concentration and allowed to complex for 30 minutes before addition to HeLa cells in 100 uL normal media containing 10% FBS. Final siRNA concentrations were evaluated at 100, 50, 25, and 12.5 nM. Polymer was added either at 4:1, 2:1 or 1:1 charge ratios, or at fixed polymer concentrations of 18, 9, 4.5, and 2.2 μg/mL to determine what conditions result in highest KD activity. For charge ratios (FIG. 17A), the complexes were prepared at higher concentrations, incubated for 30 minutes, and then serial diluted at 5-fold over concentration shown on graphs just prior to addition to cells. For fixed polymer concentration (FIG. 17B), the siRNA and polymer were complexed at 5-fold over concentrations shown on graph, incubated for 30 minutes then added to cells for final concentrations shown. FIG. 17C is the negative control. Total RNA was isolated 24 hours post treatment and GAPDH expression was measured relative to 2 internal normalizer genes, RPL13A and HPRT, by quantitative PCR. Results in FIGS. 17A, 17B, 17C and FIG. 18A and FIG. 18B indicate >60% KD activity (shading) obtained with PRx0729v6 at 9 μg/mL and higher concentrations at all siRNA concentrations tested. This concentration was coincident with stable micelle formation from particle size analyses. High KD activity was observed with 4.5 μg/mL PRx0729v6/12.5 nM siRNA only when complexes were prepared at high concentration and serial diluted (4:1 charge ratio) as compared to complex formation at lower concentration (4.5 μg/mL fixed polymer concentration). Additionally, only 100 nM siRNA with 4.5 μg/mL PRx0729v6 showed high KD activity whereas lower siRNA concentrations did not. In summary, PRx0729v6 micelles were stable to dilution down to ˜10 μg/mL and KD activity is lost below ˜5 μg/mL, indicating that stable micelles are required for good KD activity.

### Example 20

### Knock-Down Activity of Dicer Substrate GAPDH siRNA

### Polymer Complexes in Cultured Mammalian Cells

Knock-down (KD) activity of GAPDH specific dicer substrate siRNA/polymer complexes is assayed in a 96-well format by measuring GAPDH gene expression after 24 hours of treatment with polymer: GAPDH dicer siRNA complexes. The GAPDH dicer siRNA sequence is: sense strand: rGrGrUrCrArUrCrCrArUrGrArCrArArCrUrUrUrGrGrUrAdTdC, antisense strand: rGrArUrArCrCrArArArGrUrUrGrUrCrArUrGrGrArUrGrArCrCrUrU. Polymer and GAPDH targeting siRNA or negative control siRNA (IDT) are mixed in 25 uL to obtain various charge ratios and concentrations at 5-fold over final transfection concentration and allowed to complex for 30 minutes before addition to HeLa cells in 100 uL normal media containing 10% FBS. Final siRNA concentrations are examined at 100, 50, 25, and 12.5 nM. Polymer is added either at 4:1, 2:1 or 1:1 charge ratios, or at fixed polymer concentrations of 40, 20, 10, and 5 μg/mL to determine what condition results in highest KD activity. Total RNA is isolated 24 hours post treatment and GAPDH expression is measured relative to 2 internal normalizer genes, RPL13A and HPRT, by quantitative PCR. Results show >60% KD activity obtained with polymer at 10 μg/mL and higher concentrations at all siRNA concentrations tested. This polymer concentration is coincident with stable micelle formation from particle size analyses.

### Example 21

### Knock-Down Activity of ApoB100 siRNA

### Polymer Complexes in Cultured Mammalian Cells

Knock-down (KD) activity of ApoB100 specific siRNA or dicer substrate siRNA complexed to polymer is assayed in a 96-well format by evaluating ApoB100 gene expression after 24 hours of treatment with polymer: ApoB siRNA complexes. The ApoB100 siRNA sequence is: sense strand: 5′-rGrArArUrGrUrGrGrGrUrGrGrCrArArCrUrUrUrArG-3′, antisense strand: 5′-rArArArGrUrUrGrCrCrArCrCrCrArCrArUrUrCrArG-3′. The ApoB100 dicer substrate siRNA sequence is: sense strand: 5′-rGrArArUrGrUrGrGrGrUrGrGrCrArArCrUrUrUrArArArGdGdA, antisense strand: 5′-rUrCrCrUrUrUrArArArGrUrUrGrCrCrArCrCrCrArCrArUrUrCrArG-3′. Polymer and ApoB targeting siRNA or negative control siRNA (IDT) are mixed in 25 uL to obtain various charge ratios and concentrations at 5-fold over final transfection concentration and allowed to complex for 30 minutes before addition to HepG2 cells in 100 uL normal media containing 10% FBS. Final siRNA concentrations are examined at 100, 50, 25, and 12.5 nM. Polymer is added either at 4:1, 2:1 or 1:1 charge ratios, or at fixed polymer concentrations of 40, 20, 10, and 5 μg/mL to determine what condition results in highest KD activity. Total RNA is isolated 24 hours post treatment and ApoB100 expression is measured relative to 2 internal normalizer genes, RPL13A and HPRT, by quantitative PCR. Results show >60% KD activity obtained with polymer at 10 μg/mL and higher concentrations at all siRNA concentrations tested. This polymer concentration is coincident with stable micelle formation from particle size analyses.

### Example 22

### Knock-Down Activity of ApoB100 siRNA

### Polymer Complexes in a Mouse Model

The knockdown activity of ApoB100 specific siRNA/polymer complexes is determined in a mouse model by measuring ApoB100 expression in liver tissue and serum cholesterol levels. Balb/C mice are dosed intravenously via the tail vein with 1, 2 or 5 mg/kg ApoB specific siRNA complexed to polymer at 1:1, 2:1 or 4:1 charge ratio (polymer:siRNA) or saline control. 48 hours post final dose mice are sacrificed and blood and liver samples are isolated. Cholesterol levels are measured in serum. Total RNA is isolated from liver and ApoB100 expression is measured relative to 2 normalizer genes, HPRT and GAPDH by quantitative PCR. Results show >60% reduction of ApoB mRNA levels in liver at 2 mg/kg siRNA dose. This reduction is dose dependent since the 5 mg/kg siRNA dose shows >80% KD and the 1 mg/kg siRNA dose shows ˜50% KD. A reduction in serum cholesterol levels is observed, also in a dose dependent manner (˜30-50% reduction compared to saline control).

### Example 23

### Knock-Down Activity of ApoB100 Antisense DNA Oligonucleotide

### Polymer Complexes in Cultured Mammalian Cells

Knock-down (KD) capability by ApoB100 specific antisense DNA oligonucleotide complexed to polymer is assayed in a 96-well format by measuring ApoB100 gene expression after 24 hours of treatment with polymer: ApoB antisense DNA oligonucleotide complexes. Two ApoB100 antisense oligonucleotides specific to mouse ApoB are:

5′-GTCCCTGAAGATGTCAATGC-3′, position 541 of the coding region and

5′-ATGTCAATGCCACATGTCCA-3′, position 531 of the coding region

Polymer and an ApoB targeting antisense DNA oligonucleotide or negative control DNA oligonucleotide (scrambled sequence) are mixed in 25 uL to obtain various charge ratios and concentrations at 5-fold over final transfection concentration and allowed to complex for 30 minutes before addition to HepG2 cells in 100 uL normal media containing 10% FBS. Final oligonucleotide concentrations are examined at 100, 50, 25, and 12.5 nM. Polymer is added either at 4:1, 2:1 or 1:1 charge ratios, or at fixed polymer concentrations of 40, 20, 10, and 5 μg/mL to determine what condition results in the highest KD activity. Total RNA is isolated 24 hours post treatment and ApoB100 expression is measured relative to 2 internal normalizer genes, RPL13A and HPRT, by quantitative PCR.

### Example 24

### Demonstration of Membrane Destabilizing Activity of Micelles and their siRNA Complexes

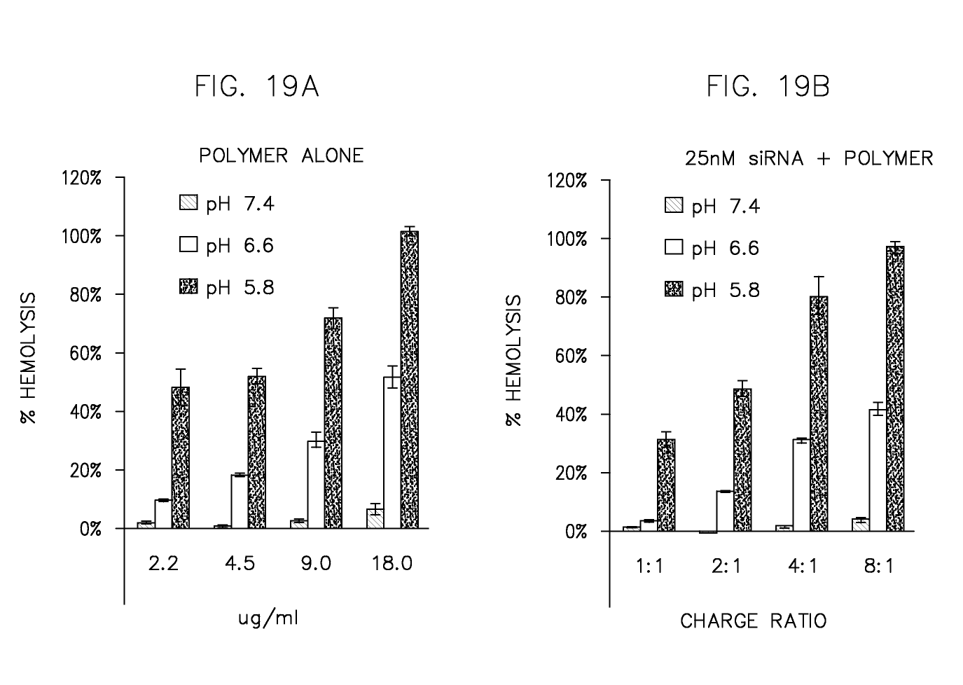

### FIG. 19

pH responsive membrane destabilizing activity was assayed by titrating polymer alone or PRx0729v6:siRNA complexes into preparations of human red blood cells (RBC) and determining membrane-lytic activity by hemoglobin release (absorbance reading at 540 nm). Three different pH conditions were used to mimic endosomal pH environments (extracellular pH=7.4, early endosome=6.6, late endosome=5.8). Human red blood cells (RBC) were isolated by centrifugation from whole blood collected in vaccutainers containing EDTA. RBC were washed 3 times in normal saline, and brought to a final concentration of 2% RBC in PBS at specific pH (5.8, 6.6 or 7.4). PRx0729v6 alone or PRx0729v6/siRNA complex was tested at concentrations just above and below the critical stability concentration (CSC) as shown (FIG. 19). For polymer/siRNA complex, 25 nM siRNA was added to PRx0729v6 at 1:1, 2:1, 4:1 and 8:1 charge ratios (same polymer concentrations for polymer alone). Solutions of polymer alone or polymer-siRNA complexes were formed at 20× final assayed concentration for 30 minutes and diluted into each RBC preparation. Two different preparations of PRx0729v6 polymer stock were compared for stability of activity at 9 and 15 days post preparation, stored at 4° C. from day of preparation. RBC with polymer alone (FIG. 19A) or polymer/siRNA complex (FIG. 19B) were incubated at 37° C. for 60 minutes and centrifuged to remove intact RBC. Supernatants were transferred to cuvettes and absorbance determined at 540 nm. Percent hemolysis is expressed as A₅₄₀ sample/A₅₄₀ of 1% Triton X-100 treated RBC (control for 100% Lysis). The results show that PRx0729v6 alone or PRx0729v6/siRNA complex is non-hemolytic at pH 7.4 and becomes increasingly more hemolytic at the lower pH values associated with endosomes and at higher concentrations of polymer.

### Example 25

### Fluorescence Microscopy of Cell Uptake and Intracellular Distribution of Polymer-siRNA Complexes

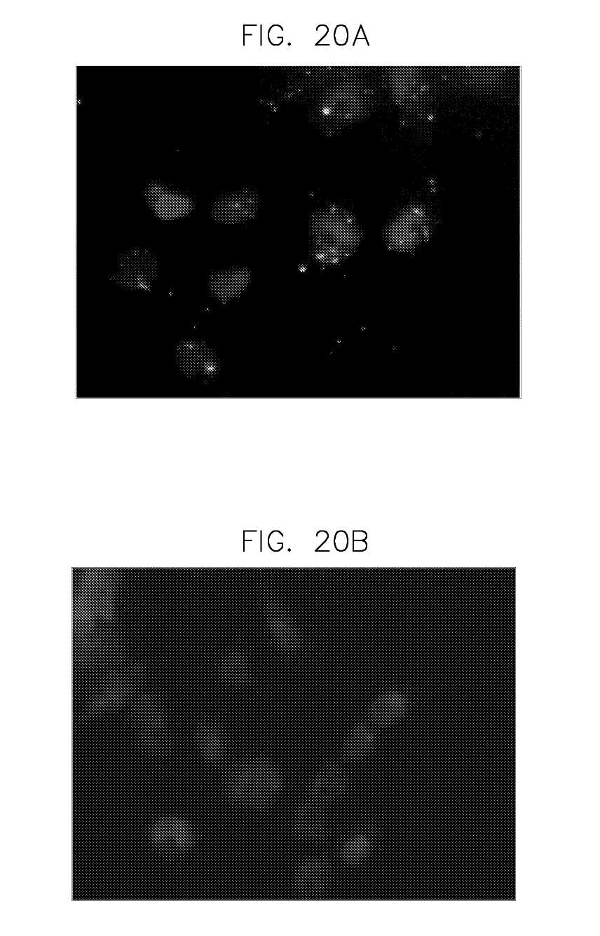

### FIG. 20

This example demonstrates that polymer PRx0729v6 can mediate a more efficient cellular uptake of fluorescent-labeled siRNA and endosomal release than a lipid-based transfection reagent.

HeLa cells were plated on a Lab-Tek II chambered coverglass. Following overnight incubation, cells were transfected with either 100 nM FAM-siRNA/lipofectamine 2000 or with 100 nM FAM-siRNA at a Polymer-siRNA 4:1 charge ratio. Complexes were formed in PBS pH 7.4 for 30 minutes at a 5× concentration, added to cells for final 1× concentration, and incubated overnight. Cells were stained with DAPI (for visualization of the nucleus) for 10 minutes and then fixed in 3.7% formaldehyde-1×PBS for 5 minutes and washed with PBS. Samples were imaged with a Zeiss Axiovert fluorescent microscope. FIG. 20B shows the fluorescence microscopy of cell uptake and intracellular distribution of polymer-siRNA compared to lipofectamine (FIG. 20A). Particulate staining of lipofectamine-siRNA complexes suggest an endosomal location, while diffuse cytoplasmic staining of polymer-siRNA complexes indicate they have been released from endosomes into the cytoplasm.

### Example 23

### Uptake of Small Hydrophobic Molecules into Polymer PRx0729v6 Micelles

This example demonstrates that small hydrophobic molecules are taken up by the predominantly hydrophobic micelle core of polymer PRx0729v6.

The formation of polymer micelles with or without siRNA is confirmed by a fluorescence probe technique using pyrene (C₁₆H₁₀, MW=202), in which the partitioning of pyrene into the micellar core could be determined using the ratio of 2 emission maxima of the pyrene spectrum. The fluorescence emission spectrum of pyrene in the polymer micelle solution is measured from 300 to 360 nm using a fixed excitation wavelength of 395 nm with a constant pyrene concentration of 6×10⁻⁷ M. The polymer varies from 0.001% to 20% (w/w) with or without 100 nM siRNA. The spectral data are acquired using a Varian fluorescence spectrophotometer. All fluorescence experiments are carried out at 25° C. The critical micelle concentration (CMC) is determined by plotting the intensity ratio I₃₃₆/I₃₃₃ as a function of polymer concentration.

Similarly, a model small molecule drug, dipyridamole (2-{[9-(bis(2-hydroxyethyl)amino)-2,7-bis(1-piperidyl)-3,5,8,10-tetrazabicyclo[4.4.0]deca-2,4,7,9,11-pentaen-4-yl]-(2-hydroxyethyl)amino}ethanol; C₂₄H₄₀N₈O₄, MW=505) is incorporated into the micelle core of PRx0729v6 as follows. Polymer (1.0 mg) and dipyridamole (DIP) (0.2 mg) are dissolved in THF (0.5 mL). Deionized water (10 mL) is added dropwise and the solution is stirred at 50° C. for 6 h to incorporate the drug into the hydrophobic core of the micelle. The solution (2.5 mL) is divided, and the absorbance of dipyridamole is measured at 415 nm by UV-vis spectroscopy at 25 and 37° C. Control measurements are also conducted by measuring the time-dependent reduction in dipyridamole absorbance in deionized water in the absence of copolymer. The absorbance at both 25 and 37° C. is measured for each time point, and the value is subtracted from that observed in the solution.

### Example 26

### Methods for Conjugating Targeting Ligands and Polynucleotides to a Copolymer

The following examples demonstrate methods for conjugating a targeting ligand (for example, galactose) or a polynucleotide therapeutic (for example siRNA) to a diblock copolymer. (1) The polymer is prepared using reversible addition fragmentation chain transfer (RAFT) (Chiefari et al. Macromolecules. 1998; 31(16):5559-5562) to form a galactose end-functionalized, diblock copolymer, using a chain transfer agent with galactose as the R-group substituent. (2) The first block of a diblock copolymer is prepared as a copolymer containing methylacrylic acid-N-hydroxy succinimide (MAA(NHS)) where a galactose-PEG-amine is conjugated to the NHS groups or where an amino-disulfide siRNA is conjugated to the NHS, or where pyridyl disulfide amine is reacted with the NHS groups to form a pyridyl disulfide that is subsequently reacted with thiolated RNA to form a polymer-RNA conjugate.

### Example 26.1

### Preparation of Galactose-PEG-Amine and Galactose-CTA

Scheme 1 illustrates the synthesis scheme for galactose-PEG-amine (compound 3) and the galactose-CTA (chain transfer agent) (compound 4).

Compound 1: Pentaacetate galactose (10 g, 25.6 mmol) and 2-[2-(2-Chloroethoxy)ethoxy]ethanol (5.6 mL, 38.4 mmol) were dissolved in dry CH₂Cl₂ (64 mL) and the reaction mixture was stirred at RT for 1 h. The BF₃.OEt₂ (9.5 ml, 76.8 mmol) was added to the previous mixture dropwise over 1 h in an ice bath. The reaction mixture was stirred at room temperature (RT) for 48 h. After the reaction, 30 mL of CH₂Cl₂ was added to dilute the reaction. The organic layer was neutralized with saturated NaHCO_{3(aq)}, washed by brine and then dried by MgSO₄. The CH₂Cl₂ was removed under reduced pressure to get the crude product. The crude product was purified by flash column chromatography to get final product 1 as slight yellow oil. Yield: 55% TLC (I₂ and p-Anisaldhyde): EA/Hex:1/1 (Rf: β=0.33; α=0.32; unreacted S.M 0.30).

Compound 2: Compound 1 (1.46 g, 2.9 mmol) was dissolved in dry DMF (35 mL) and the NaN₃ (1.5 g, 23.2 mmol) was added to the mixture at RT. The reaction mixture was heated to 85-90 C overnight. After the reaction, EA (15 mL) was added to the solution and water (50 mL) was used to wash the organic layer 5 times. The organic layer was dried by MgSO₄ and purified by flash column chromatography to get compound 2 as a colorless oil. Yield: 80%, TLC (I₂ and p-Anisaldhyde): EA/Hex:1/1 (Rf: 0.33).

Compound 3: Compound 2 (1.034 g, 2.05 mmol) was dissolved in MeOH (24 mL) and bubbled with N₂ for 10 min and then Pd/C (10%) (90 mg) and TFA (80 uL) were added to the previous solution. The reaction mixture was bubbled again with H₂ for 30 min and then the reaction was stirred at RT under H₂ for another 3 h. The Pd/C was removed by celite and MeOH was evaporated to get the compound 3 as a sticky gel. Compound 3 can be used without further purification. Yield: 95%. TLC (p-Anisaldhyde): MeOH/CH₂Cl₂: 1/4 (Rf: 0.05).

Compound 4: ECT (0.5 g, 1.9 mmol), NHS (0.33 g, 2.85 mmol) and DCC (0.45 g, 2.19 mmol) were dissolved in CHCl₃ (15 mL) at 0 C. The reaction mixture was continuously stirred at RT overnight. Compound 3 (1.13 g, 1.9 mmol) and TEA (0.28 mL, 2.00 mmol) in CHCl₃ (10 mL) were added slowly to the previous reaction at 0 C. The reaction mixture was continuously stirred at RT overnight. The CH₃C1 was removed under reduced pressure and the crude product was purified by flash column chromatography to get the compound 4 as a yellow gel. Yield (35%). TLC: MeOH/CH₂Cl₂: 1/9 (Rf: 0.75)

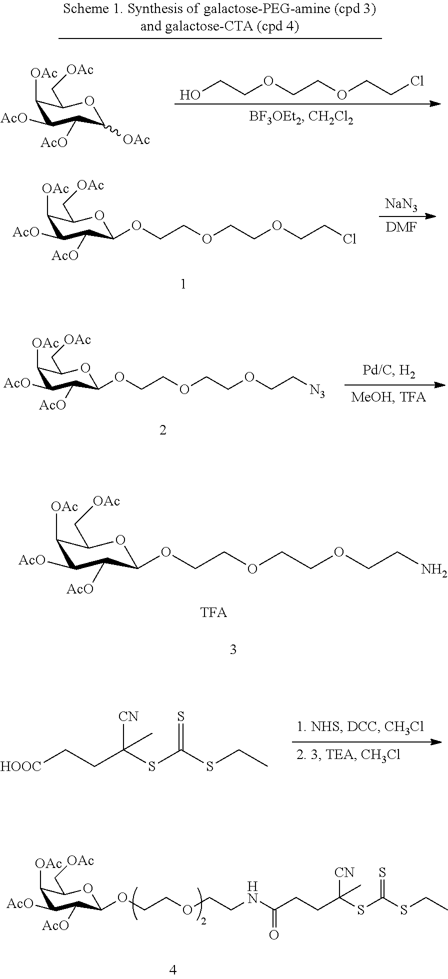

### Example 26.2

### Synthesis of [DMAEMA]-[BMA-PAA-DMAEMA]

### A. Synthesis of DMAEMA MacroCTA.

Polymerization: In a 20 mL glass vial (with a septa cap) was added 33.5 mg ECT (RAFT CTA), 2.1 mg AIBN (recrystallized twice from methanol), 3.0 g DMAEMA (Aldrich, 98%, was passed through a small alumina column just before use to remove the inhibitor) and 3.0 g DMF (high purity without inhibitor). The glass vial was closed with the Septa Cap and purged with dry nitrogen (carried out in an ice bath under stirring) for 30 min. The reaction vial was placed in a preheated reaction block at 70° C. The reaction mixture was stirred for 2 h 40 min. The septa cap was opened and the mixture was stirred in the vial in an ice bath for 2-3 minutes to stop the polymerization reaction.

Purification: 3 mL of acetone was added to the reaction mixture. In a 300 mL beaker was added 240 mL hexane and 60 mL ether (80/20 (v/v)) and under stirring the reaction mixture was added drop by drop to the beaker. Initially this produces an oil which is collected by spinning down the cloudy solution; yield=1.35 g (45%). Several precipitations were performed (e.g., 6 times) in hexane/ether (80/20 (v/v)) mixed solvents from acetone solution Finally, the polymer was dried under vacuum for 8 h at RT; yield≈1 g.

Summary: (N_{n,theory}=11,000 g/mol at 45% conv.)

| Name | FW (g/mol) | Equiv. | mol | Weight | | Actual weight | |
|---|---|---|---|---|---|---|---|
| DMAEMA | 157.21 | 150 | 0.0191 | 3.0 | g | 3.01 | g |
| ECT | 263.4 | 1 | 1.2722 × 10⁻⁴ | 33.5 | mg | 33.8 | mg |
| AIBN | 164.21 | 0.1 | 1.2722 × 10⁻⁵ | 2.1 | mg | 2.3 | mg |

DMF=3.0 g; N₂ Purging: 30 min; Conduct polymerization at 70° C. for 2 h 45 min.

B. Synthesis of [BMA-PAA-DMAEMA] from DMAEMA MacroCTA

All chemicals and reagents were purchased from Sigma-Aldrich Company unless specified. Butyl methacrylate (BMA) (99%), 2-(Dimethylamino) ethyl methacrylate (DMAEMA) (98%) were passed through a column of basic alumina (150 mesh) to remove the polymerization inhibitor. 2-propyl acrylic acid (PAA) (>99%) was purchased without inhibitor and used as received. Azobisisobutyronitrile (AIBN) (99%) was recrystallized from methanol and dried under vacuum. The DMAEMA macroCTA was synthesized and purified as described above (Mn˜10000; PDI˜1.3; >98%). N,N-Dimethylformamide (DMF) (99.99%) (Purchased from EMD) was reagent grade and used as received. Hexane, pentane and ether were purchased from EMD and they were used as received for polymer purification.

Polymerization: BMA (2.1 g, 14.7 mmoles), PAA (0.8389 g, 7.5 mmoles), DMAEMA (1.156 g, 7.35 mmoles), MacroCTA (0.8 g, 0.0816 mmoles), AIBN (1.34 mg, 0.00816 mmoles; CTA:AIBN 10:1) and DMF (5.34 ml) were added under nitrogen in a sealed vial. The CTA:Monomers ratio used was 1:360 (assuming 50% of conversion). The monomers concentration was 3 M. The mixture was then degassed by bubbling nitrogen into the mixture for 30 minutes and then placed in a heater block (Thermometer: 67° C.; display: 70-71; stirring speed 300-400 rpm). The reaction was left for 6 hours, then stopped by placing the vial in ice and exposing the mixture to air.

Purification: Polymer purification was done from acetone/DMF 1:1 into hexane/ether 75/25 (three times). The resulting polymer was dried under vacuum for at least 18 hours. The NMR spectrum showed a high purity of the polymer. No vinyl groups were observed. The polymer was dialysed from ethanol against double de-ionized water for 4 days and then lyophilized. The polymer was analyzed by gel permeation chromatography (GPC) using the following conditions: Solvent: DMF/LiBr 1%. Flow rate: 0.75 ml/min. Injection volume: 100 μl.

Column temperature: 60° C. Poly(styrene) was used to calibrate the detectors. GPC analysis of the resulting Polymer: Mn=40889 g/mol. PDI=1.43. dn/dc=0.049967.

### Example 26.3

### Synthesis of Gal-[DMAEMA]-[BMA-PAA-DMAEMA]

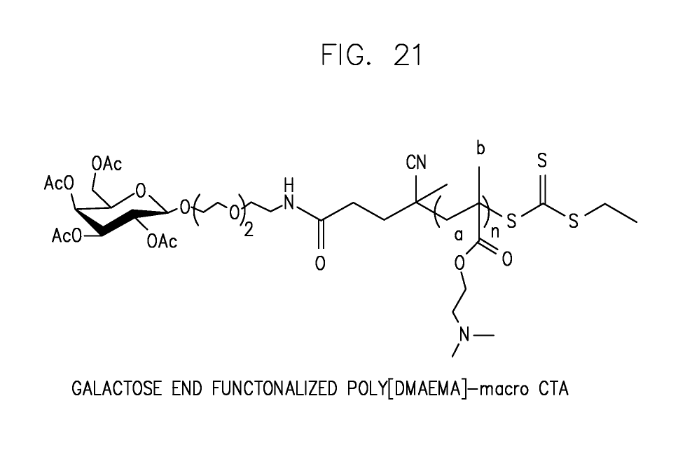

Synthesis was carried out as described in example 20.2. First, a galactose-DMAEMA macro-CTA was prepared (example 20.2.A.) except that galactose-CTA (example 20.1, cpd 4) was used in place of ECT as the chain transfer agent. This resulted in the synthesis of a polyDMAEMA with an end functionalized galactose (FIG. 21). The galactose-[DMAEMA]-macro-CTA was then used to synthesize the second block [BMA-PAA-DMAEMA] as described in example 20.2.B. Following synthesis, the acetyl protecting groups on the galactose were removed by incubation in 100 mM sodium bicarbonate buffer, pH 8.5 for 2 hrs, followed by dialysis and lyophilization. NMR spectroscopy was used to confirm the presence of the deprotected galactose on the polymer.

### Example 26.4

### Preparation and Characterization of [PEGMA-MAA(NHS)]-[B—P-D] and DMAEMA-MMA(NHS)-[B—P-D] Diblock Co-Polymers

Polymer synthesis was performed as described in example 20.2 (and summarized in FIG. 5) using monomer feed ratios to obtain the desired composition of the 1^{st} block copolymer. FIG. 6 summarizes the synthesis and characterization of [PEGMA-MAA(NHS)]-[B—P-D] polymer where the co-polymer ratio of monomers in the 1^{st} block is 70:30.

### Example 26.5

### Conjugation of Galactose-PEG-Amine to PEGMA-MAA(NHS) to Produce [PEGMA-MAA(Gal)]-[B—P-D] Polymer

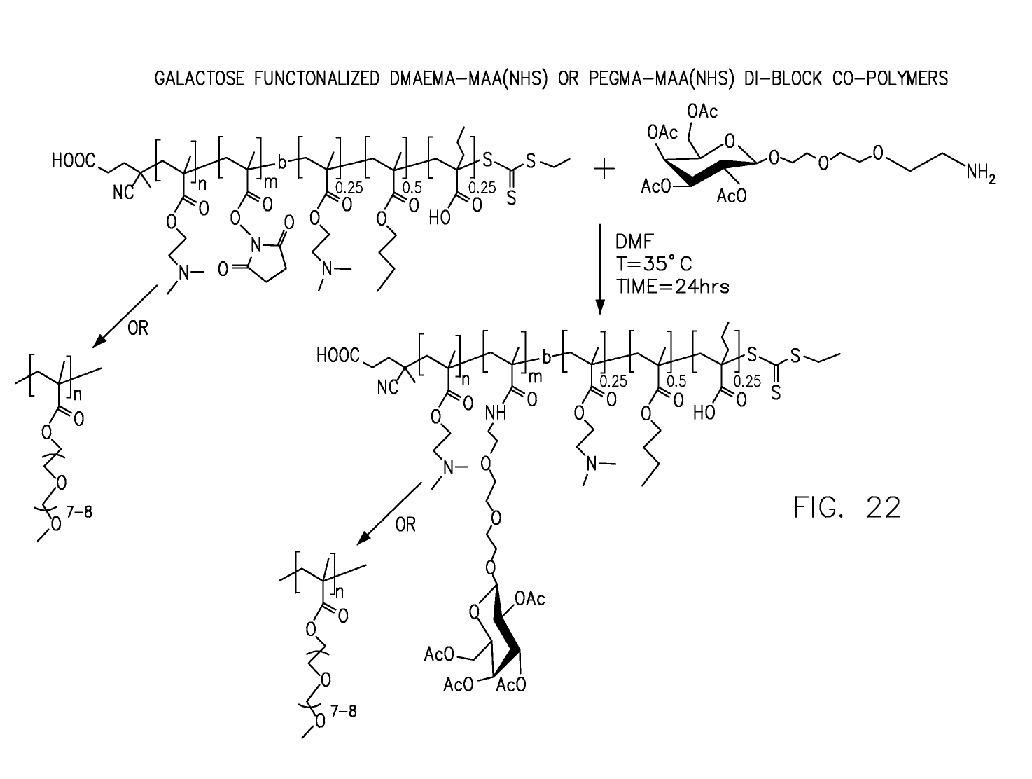

FIG. 22 illustrates the preparation of galactose functionalized DMAEMA-MAA(NHS) or PEGMA-MAA(NHS) di-block co-polymers. Polymer [DMAEMA-MAA(NHS)]-[B—P-D] or [PEGMA-MAA(NHS)]-[B—P-D] was dissolved in DMF at a concentration between 1 and 20 mg/mL. Galactose-PEG-amine prepared as described in example 20.1 (cpd 3) was neutralized with 1-2 equivalents of triethylamine and added to the reaction mixture at a ratio of 5 to 1 amine to polymer. The reaction was carried at 35° C. for 6-12 hrs, followed by addition of an equal volume of acetone, dialysis against deionized water for 1 day and lyophilization.

### Example 26.6

### Conjugation of siRNA to PEGMA-MAA(NHS)]—[B—P-D] to produce [PEGMA-MAA(RNA)]-[B—P-D] polymer

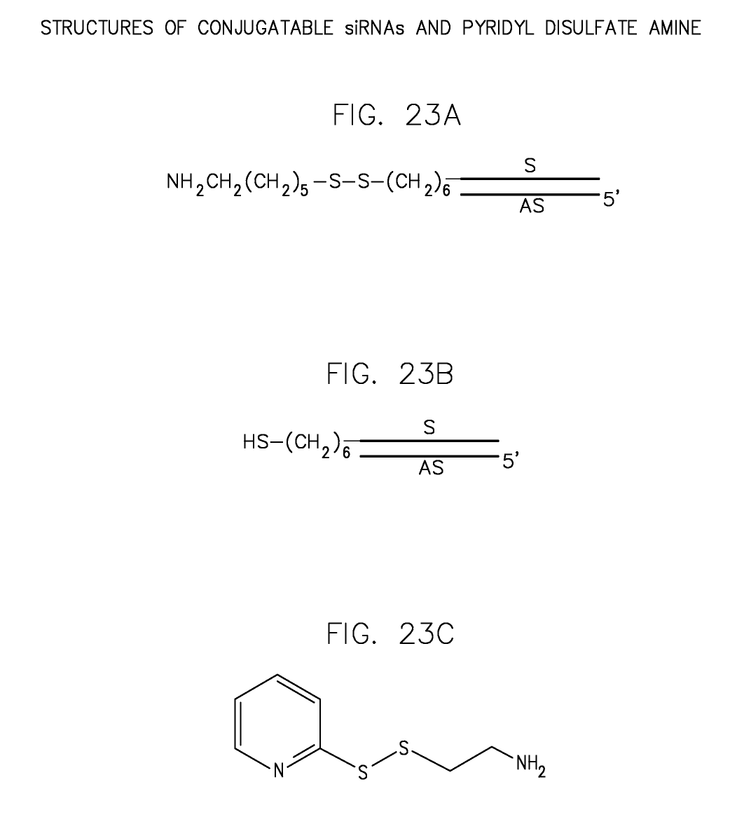

FIG. 23 A and FIG. 23 B shows the structures of 2 modified siRNAs that can be conjugated to NHS containing polymers prepared as described in example 20.4. siRNAs were obtained from Agilent (Boulder, Colo.). FIG. 23 C shows the structure of pyridyl disulfide amine used to derivatize NHS containing polymers to provide a disulfide reactive group for the conjugation of thiolated RNA (FIG. 23 B).

Reaction of NHS containing polymer with amino-disulfide-siRNA. The reaction is carried out under standard conditions consisting of an organic solvent (for example, DMF or DMSO, or a mixed solvent DMSO/buffer pH 7.8.) at 35° C. for 4-8 hrs, followed by addition of an equal volume of acetone, dialysis against deionized water for 1 day and lyophilization.

Reaction of NHS containing polymer with pyridyl-disulfide-amine and reaction with thiolated siRNA. Reaction of pyridyl disulfide amine with NHS containing polymers is carried out as described in example 20.5. Subsequently the lyophilized polymer is dissolved in ethanol at 50 mg/mL and diluted 10-fold in sodium bicarbonate buffer at pH 8. Thiolated siRNA (FIG. 23 B) is reacted at a 2-5 molar excess over polymer NHS groups at 35° C. for 4-8 hrs, followed by dialysis against phosphate buffer, pH 7.4.

### Example 27

### Determination of Micelle Aggregation Number

### Polymer Chains Per Micelle

The weight average molecular weight (Mw) and the aggregation number (N_{aggr}) of the micelles were determined by static light scattering (SLS) measurements using a Debye plot. This method assumes that the intensity of scattered light that a particle produces is proportional to the product of the weight-average molecular weight and the concentration of the particle, as represented by the following equation:

$$\frac{KC}{{R}_{θ}} = ( \frac{1}{M} + 2 {A}_{2} C )$$

Where R_{θ} is the Rayleigh ratio (ratio of scattered light to incident light of the sample); M is the sample molecular weight; A₂ is the 2^{nd} Viral Coefficient; C is the concentration; K is the optical constant defined as K=4¶²(n₀ dn/dc)²/λ₀⁴N_{A}, where N_{A} is Avogadro's number; λ₀ is the laser wavelength; n₀ is the solvent refractive index; and dn/dc is the differential refractive index increment of the micelles (0.2076 ml/g).

The measurement of the intensity of scattered light (K/CR) of various concentrations (C) of polymers at one angle was determined using a Malvern Zetasizer Nano ZS instrument and compared with the scattering produced from a standard (i.e. Toluene). The Debye plot is a straight line and allows the determination of the absolute molecular weight of the micelles which is the y intercept of the plot at zero concentration (K/CR=1/M_{W} in Daltons). The aggregation number was calculated by dividing the molecular weight of the micelles (determined from the Debye plot) with the molecular weight of the single polymer chain (calculated by GPC-triple detection method). Typical values range from 30 to 50 for diblock polymers, for example, [D]_{10K}-[B₅₀—P₂₅-D₂₅]_{20-66K}.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-49. (canceled)

50. A composition comprising a polymeric micelle and a polynucleotide associated with the micelle, the micelle comprising a plurality of block copolymers, each block copolymer comprising a hydrophilic block and a hydrophobic block, the plurality of block copolymers associating such that the micelle is stable in an aqueous medium at about neutral pH,
(a) the micelle further having two or more characteristics selected from:
(i) the micelle comprising from about 10 to about 100 of the block copolymers per micelle,
(ii) a critical micelle concentration, CMC, ranging from about 0.21 μg/mL to about 20 μg/mL,
(iii) spontaneous micelle assembly in the absence of nucleic acid;
(iv) a weight average molecular weight of about 0.5×106 to about 3.6×106 dalton;
(v) a particle size of about 5 nm to about 500 nm; and
(b) the block copolymers having one or more characteristic selected from:
(i) a ratio of a number-average molecular weight, Mn, of the hydrophilic block to the hydrophobic block, ranging from about 1:1 to about 1:10, and
(ii) a polydispersity index of about 1.0 to about 2.0.

51. The composition of claim 50, wherein the micelle has all of the characteristics of subparagraphs (i), (ii), (iii) and (iv) thereof.

52. The composition of claim 50, wherein the block copolymer has a ratio of a number-average molecular weight, Mn, of the hydrophilic block to the hydrophobic block, ranging from about 1:1.5 to about 1:6.

53. The composition of claim 50, wherein the micelle comprises about 10 to about 100 of the block copolymers per micelle.

54. The composition of claim 50, wherein the micelle is has a critical micelle concentration, CMC, of about 0.2 μg/mL to about 20 μg/mL.

55. The composition of claim 50, wherein the block copolymer has a ratio of a number-average molecular weight, Mn, of the hydrophilic block to the hydrophobic block, ranging from about 1:1.5 to about 1:6; and the micelle
(i) comprises about 20 to about 60 of the block copolymers per micelle, and
(ii) has a critical micelle concentration, CMC, of about 0.5 μg/mL to about 10 μg/mL.

56. The composition of claim 50, wherein the block copolymers have a polydispersity index of about 1.0 to about 1.7.

57. The composition of claim 50, wherein the micelle has an weight average molecular weight, Mw, of about 0.75×106 to about 2.0×106.

58. The composition of claim 50, wherein the micelle comprises a block copolymer comprising a plurality of cationic monomeric units, the cationic species in the hydrophilic block being in ionic association with the polynucleotide.

59. The composition of claim 58, wherein the cationic monomeric units are residues of cationic monomers, non-charged Brønsted base monomers, or a combination thereof.

60. The composition of claim 50, wherein the polynucleotide is not in the core of the micelle.

61. The composition of claim 50, wherein the micelle comprises a block copolymer comprising a plurality of anionic monomeric units in the hydrophilic block and/or the hydrophobic block.

62. The composition of claim 50, wherein the micelle comprises a block copolymer comprising a plurality of uncharged monomeric units in the hydrophilic block and/or the hydrophobic block.

63. The composition of claim 50, comprising one or more polynucleotides covalently coupled to one or more of the plurality of block copolymers.

64. The composition of claim 63, wherein the polynucleotide is an siRNA.

65. The composition of claim 50, wherein the micelle comprises a block copolymer comprising a plurality of monomeric units having a protonatable anionic species and a plurality of hydrophobic species.

66. The composition of claim 65, wherein the monomeric units are residues of anionic monomers, non-charged Brønsted acid monomers, or a combination thereof.

67. The composition of claim 50, wherein the micelle comprises a block copolymer comprising a plurality of monomeric units derived from a polymerizable monomer having a hydrophobic species.

68. The composition of claim 50, wherein the one or more of the block copolymers is a membrane destabilizing block copolymer.

69. The composition of claim 50, wherein the number of polynucleotides associated with each micelle is about 1 to about 10,000.

70. A method for intracellular delivery of a polynucleotide, comprising contacting a cell with the composition of claim 50.
